(19) United States
(12) Patent Application Publication
(10) Pub. No.: US 2013/0225529 A1
(43) Pub. Date: Aug. 29, 2013

(54) PHOSPHO-ESTER DERIVATIVES AND USES THEREOF

(71) Applicant: Basil RIGAS, Setauket, NY (US)

(72) Inventor: Basil RIGAS, Setauket, NY (US)

(21) Appl. No.: 13/779,382

(22) Filed: Feb. 27, 2013

Related U.S. Application Data
(60) Provisional application No. 61/603,536, filed on Feb. 27, 2012.
(60) Provisional application No. 61/703,980, filed on Sep. 21, 2012.

(51) Int. Cl. A61K 31/661; A61K 45/06; A61K 31/192; A61K 31/675; A61K 31/664

(52) U.S. Cl. CPC A61K 31/661; A61K 31/675; A61K 31/664; A61K 31/192; A61K 45/06
USPC 514/91; 514/109; 514/119; 514/120; 514/569

(57) ABSTRACT

Phospho-ester compounds and pharmaceutical compositions thereof administered by the respiratory and other routes for the prevention and/or treatment of lung and brain cancer and precancerous conditions thereof, for the treatment of pain, for the treatment of skin disorders, for treating and/or preventing inflammation-related diseases, and for the treatment and prevention of cancer.

### CROSS REFERENCE TO RELATED APPLICATIONS

This application claims benefit of U.S. Provisional Application Nos. 61/603,536 and 61/703,980, which were filed on Feb. 27, 2012 and Sep. 21, 2012, respectively, and each of which is hereby incorporated by reference in its entirety.

The invention relates to compounds and pharmaceutical compositions for the prevention and/or treatment of lung and brain cancer and precancerous conditions thereof, for the treatment of pain, for the treatment of skin disorders, for treating and/or preventing inflammation-related diseases, and for the treatment and prevention of cancer.

### BACKGROUND OF THE INVENTION

Genital warts are benign skin tumors caused by infection with human papilloma virus (HPV), the most common sexually transmitted virus in the Western world. Of genital warts, 90% are caused by HPV 6 or 11. The estimated prevalence rate of HPV genital infection in the US adult population is 10-20 percent (Fleischer A B, Parrish C A, Glenn R, Feldman S R: Condylomata acuminata (genital warts): patient demographics and treating physicians. Sex Transm Dis. 2001; 28: 643-7). The prevalence of clinical manifestations of HPV genital infection is estimated to be 1 percent in the sexually active population.

Known topical treatments of genital warts include podophyllin resin, imiquimod, trichloroacetic acid, and podophyllotoxin. Surgical or destructive therapies include carbon dioxide laser, surgical excision, loop excision, cryotherapy, and electrodesiccation. There are also systemic treatments of warts that involve interferon (IFN), retinoids (isotretinoin), and cimetidine.

There is still a need for new therapeutic treatments of genital warts and other non-cancerous skin disorders, for example hirsutism, actinic keratosis, and eczema.

Pain is the most common symptom for which patients seek medical assistance. In the case of incurable diseases, treatment for pain may last for extended periods of time. Although subjective, most pain is associated with tissue damage and has a physiological basis. Pain can be either acute or chronic. Acute pain is generally caused by sudden injury, tissue damage, or infection for which the cause is easily found. Chronic pain, however, is the pain of pathological conditions and often difficult to isolate and treat. Chronic pain is routinely defined as pain of over six months' duration.

For patients suffering from chronic pain, the autonomic nervous system adapts to the pain and evidences of autonomic hyperactivity such as tachycardia, hypertension, diaphoresis, mydriasis, and pallor disappear, leaving the physician to rely on the patient's subjective complaints in assessing chronic pain.

Analgesics are drugs used to decrease pain without causing loss of consciousness or sensory perception. There are two basic classes of analgesics: anti-inflammatory, routinely prescribed for short-term pain relief and for modest pain, and opioids used for either short-term or long term pain relief of severe pain. The anti-inflammatory analgesics generally provide analgesia, anti-inflammation, and antipyretic action. It has been reported that the mechanism of action may be to provide inhibition of the synthesis of prostaglandins.

The opioid analgesics, or narcotics, include all natural or synthetic chemical compounds closely related to morphine and are thought to activate one or more receptors on brain neurons. Opioid analgesics have serious side effects and thus are to be used with caution. These side effects include: 1) tolerance, which requires gradually increasing doses to maintain analgesia; 2) physical dependence, which means that the narcotics must be withdrawn gradually if they are discontinued after prolonged use; 3) constipation, which requires careful attention to bowel function, including use of stool softeners, laxatives, and enemas; and 4) various degrees of somnolence, or drowsiness, which requires adjustments in dosages and dose scheduling, or possibly varying the type of narcotic to find one better tolerated by the patient.

Lung cancer is a major cause of cancer mortality in the industrial world. Despite significant advances in its early detection, the survival of lung cancer patients remains poor. Because of frequent and widespread metastases, surgical procedures for lung cancer are not particularly effective and therefore chemotherapy often is the treatment of choice. The efficacy of chemotherapy against lung cancer is, however, limited primarily by the intrinsically low anticancer activity of available agents; the development of drug resistance; and drug toxicity. Therefore, there is a pressing need for the development not only of new drugs but also of methods of their administration to treat lung cancer and its precancerous conditions.

An important approach to the control of lung cancer is the form of cancer prevention known as chemoprevention, i.e., the administration of natural or synthetic agents to subjects at risk of cancer to prevent its development or its recurrence in those who already had a cancer. When effective, chemoprevention abrogates the development of lung cancer. Prominent among those individuals at risk of lung cancer are former and current smokers, and those with its precancerous conditions. The opportunity for the chemoprevention of lung cancer is provided by the fact that the development of lung cancer represents a long transition of the tracheal epithelium from normal through various precancerous stages to lung cancer. Therefore, chemoprevention (administered during this transitional period) is a simpler and more cost-effective approach compared to treating an already developed lung cancer.

Regarding the treatment and/or prevention of lung cancer and its precancerous conditions, there is a need for a) new anticancer drugs and b) improved methods to administer such drugs, which would enhance their delivery to the lung and limit systemic drug exposure, thus reducing side effects.

New compounds are needed for the prevention and/or treatment of lung and brain cancer, precancerous conditions, pain, skin disorders, inflammation-related diseases, and cancer.

### SUMMARY OF THE INVENTION

The present invention features compounds and therapies for prevention and/or treatment of conditions, such as cancer (i.e., lung or brain cancer and precancerous conditions), pain, inflammation, and skin conditions.

In a first aspect the invention features a compound of general Formula I

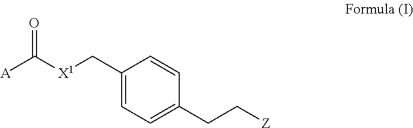

or a pharmaceutically acceptable salt thereof.

In Formula I: A is an optionally substituted aliphatic, heteroaliphatic, aromatic, heteroaromatic substituent or alkylaryl substituent having 1 to 100 carbon atoms or is selected from:

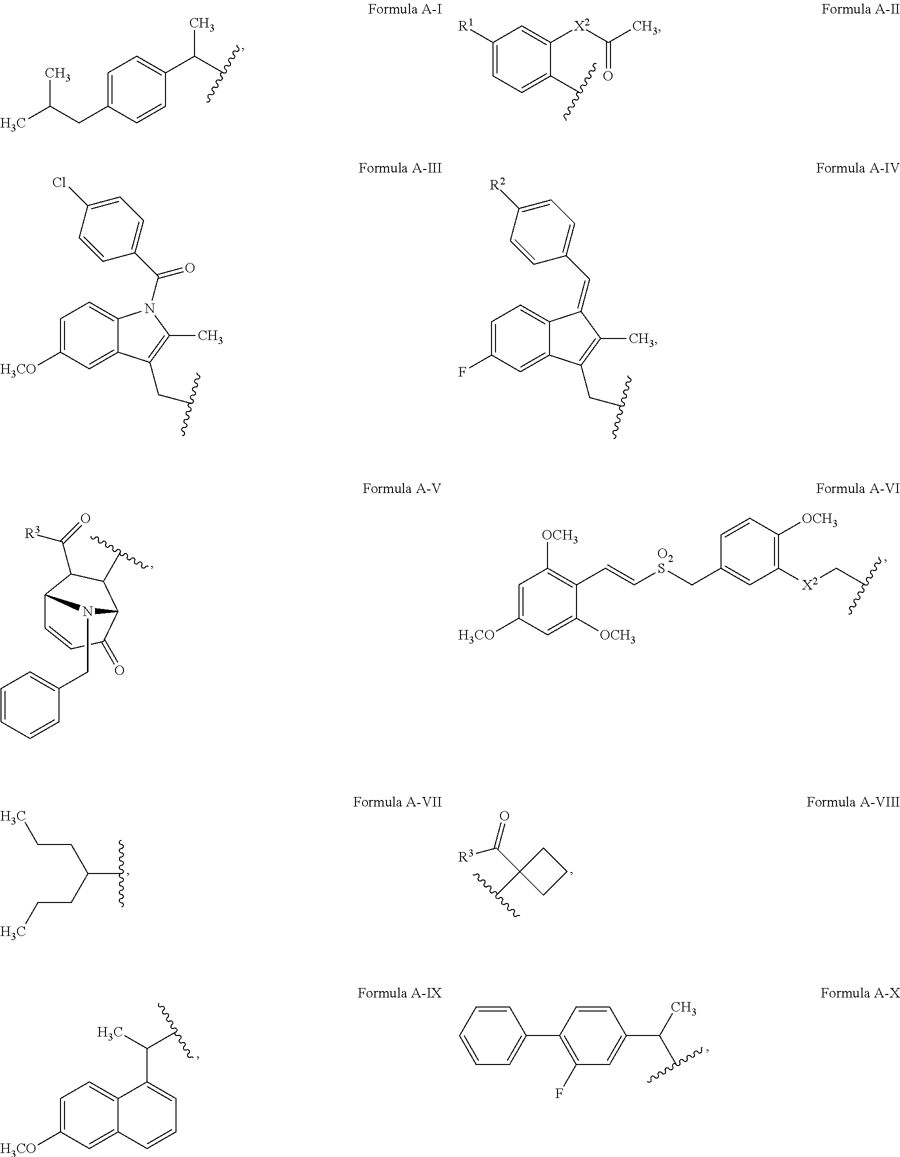

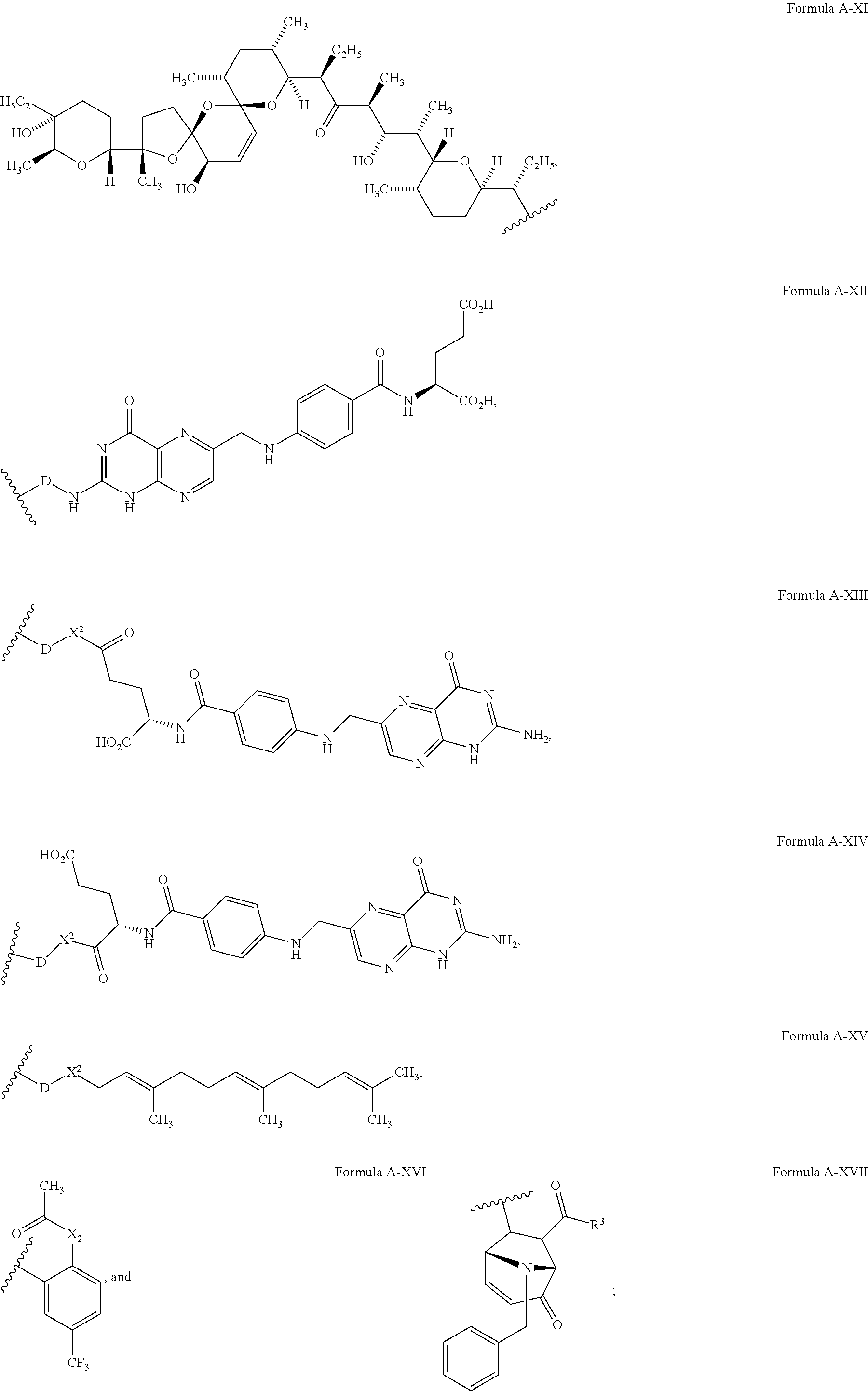

D is absent or

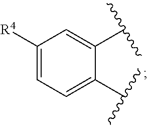

X¹ and X² are independently selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, —X¹—(CH₂)₄—Z, and

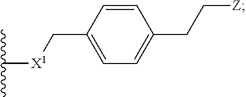

R⁵ is selected from hydrogen and C₁₋₆ alkyl;

Z is selected from:

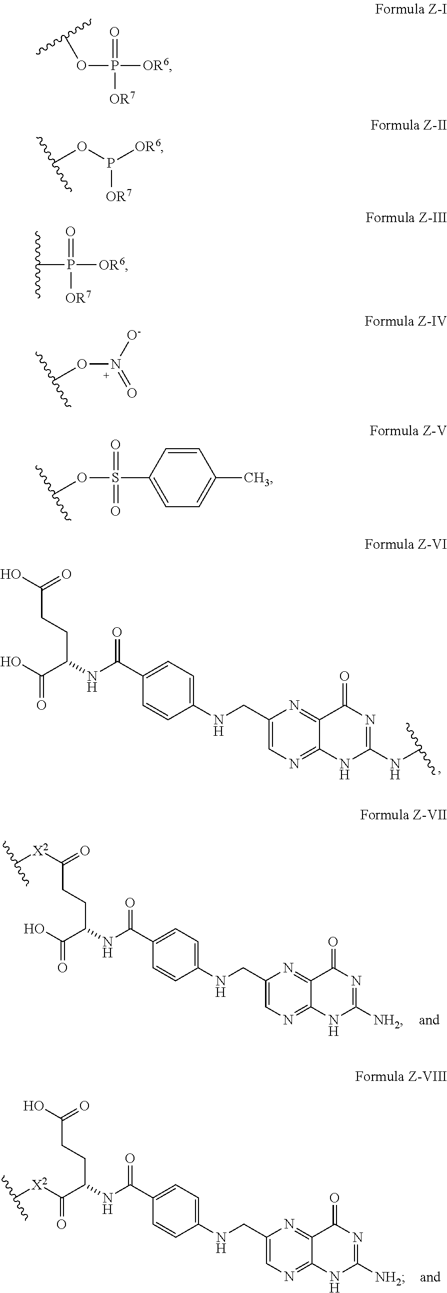

R⁶ and R⁷ are independently selected from hydrogen, C₁₋₆-alkyl, and polyethylene glycol residue.

In some embodiments, X¹ is —NR⁵—, and R⁵ is selected from hydrogen, methyl, and ethyl.

In other embodiments, X¹ is —O—.

In certain embodiments, Z is

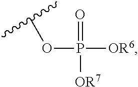

R⁶ is selected from ethyl and a polyethylene glycol residue, and R⁷ is selected from hydrogen and ethyl.

In still other embodiments, A is selected from:

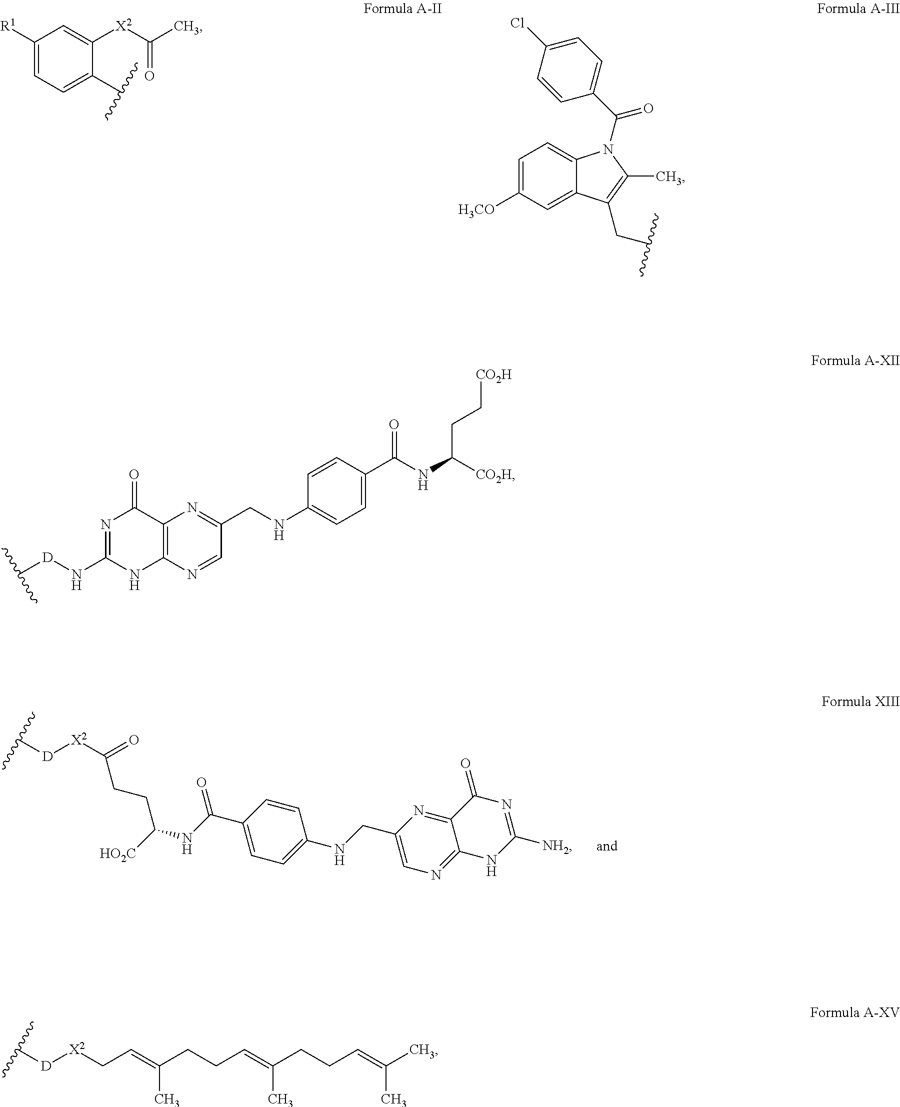

wherein D is

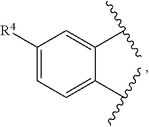

R¹ and R⁴ are independently selected from hydrogen and trifluoromethyl, and X² is selected from —O—, —S—, and —NH—.

In some embodiments, X¹ is —O—, Z is —O—P(O)(CH₂CH₃)₂, and A is:

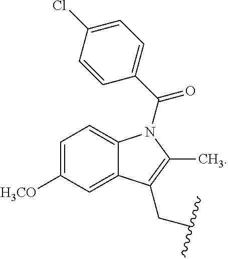 [SMILES: *Cc1c(C)n(C(=O)c2ccc(Cl)cc2)c2ccc(OC)cc12]

In certain embodiments, X¹ is selected from —O— and —NH—, Z is —O—P(O)(CH₂CH₃)₂, A is:

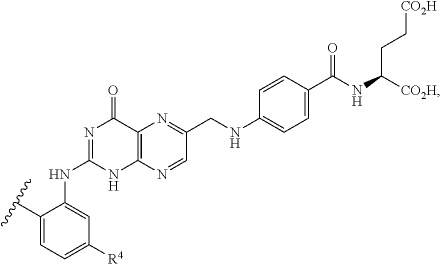

and R⁴ is selected from hydrogen and trifluoromethyl.

In other embodiments, X¹ and X² are independently selected from —O— and —NH—, Z is —O—P(O)(CH₂CH₃)₂, A is:

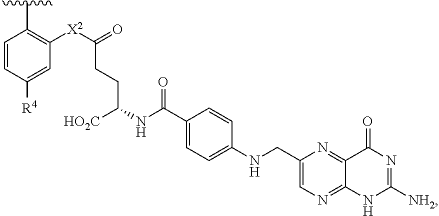

and R⁴ is selected from hydrogen and trifluoromethyl.

In some embodiments, X¹ and X² are independently selected from —O—, —S—, and —NH—; Z is —O—P(O)(CH₂CH₃)₂; and A is:

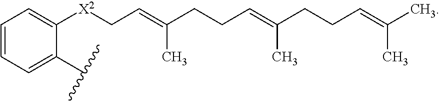

In some embodiments, X¹ is selected from —O—, —S—, and —NH—, Z is selected from —O—P(O)(CH₂CH₃)₂ and —ONO₂, A is:

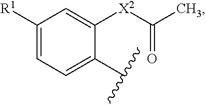

and R¹ is selected from hydrogen and trifluoromethyl, and X² is selected from —O—, —S— and —NH—.

In certain embodiments, X¹ is selected from —O— and —NH—, Z is —ONO₂, and A is:

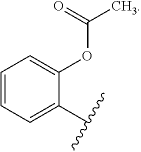 [SMILES: *c1ccccc1OC(C)=O]

Accordingly, the compounds of Formula I include but are not limited to compounds of which the structures are shown below:

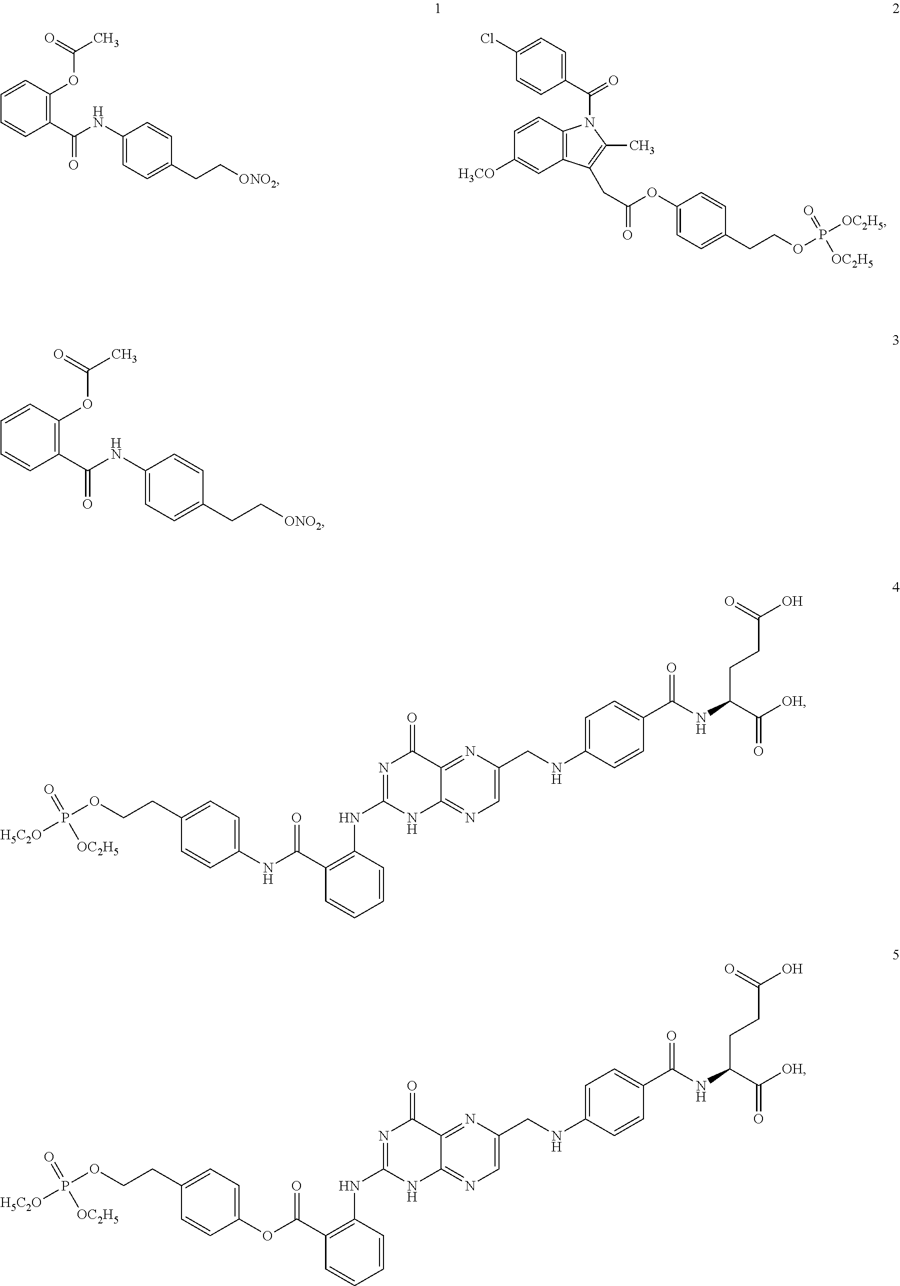

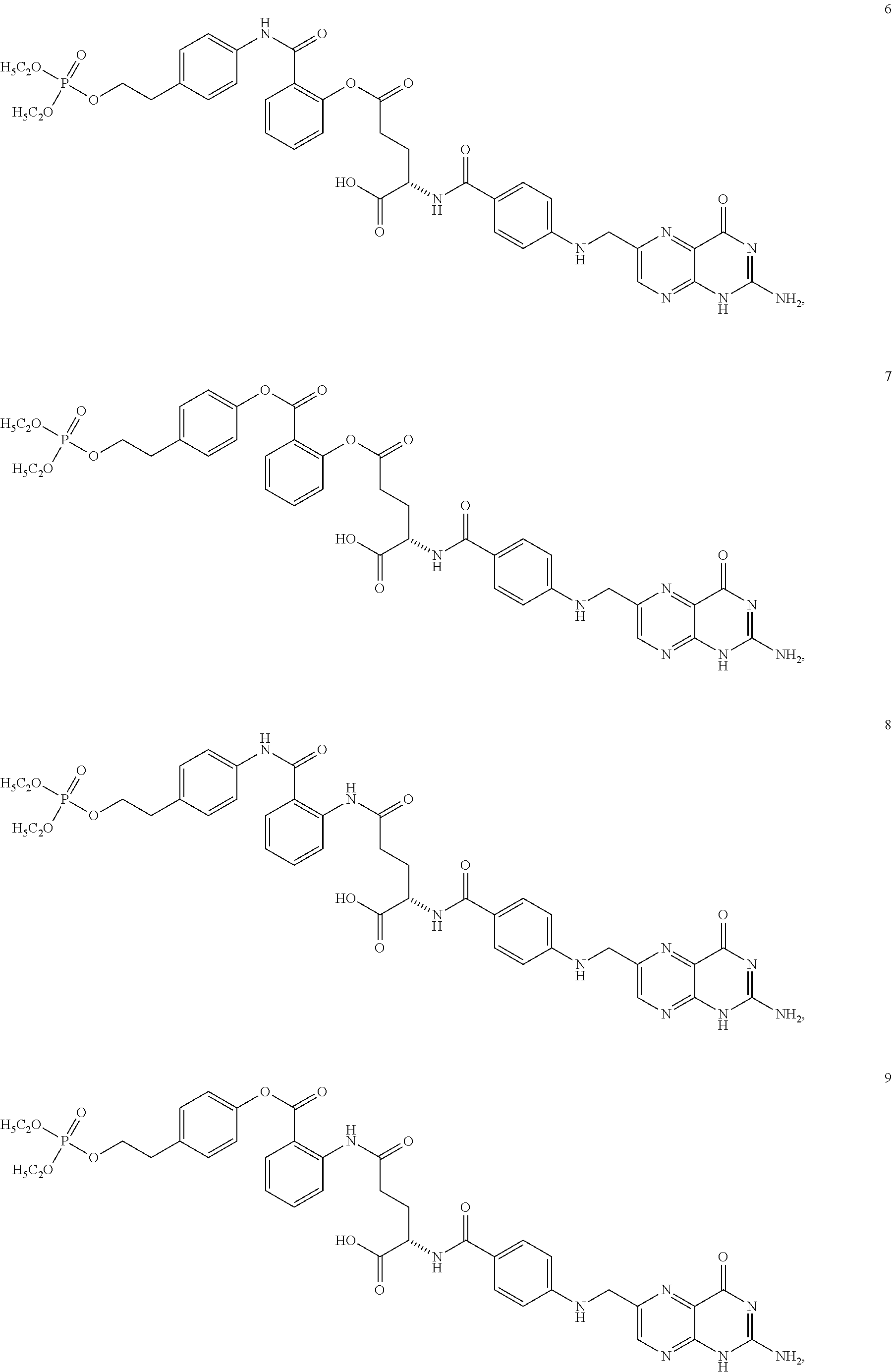

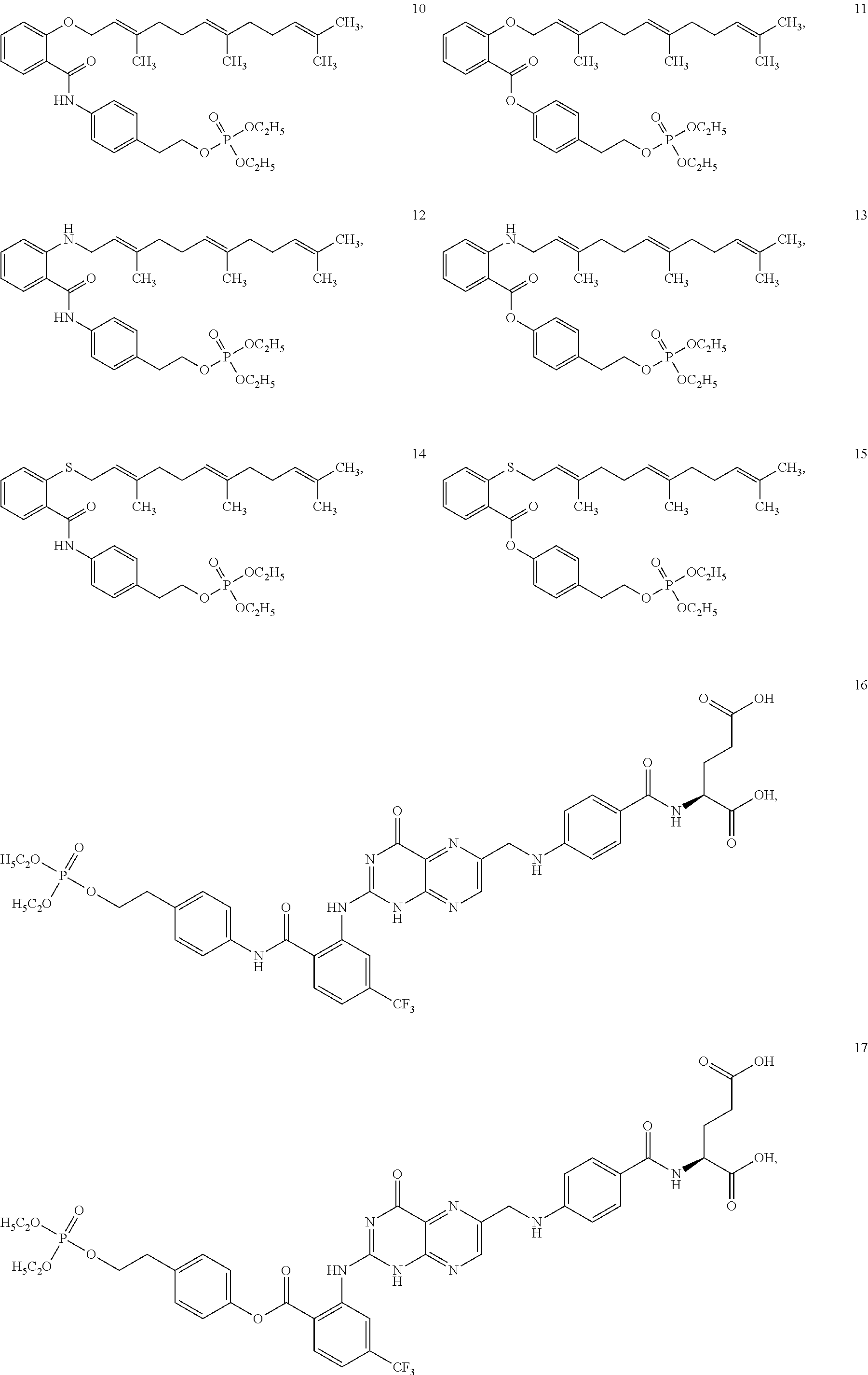

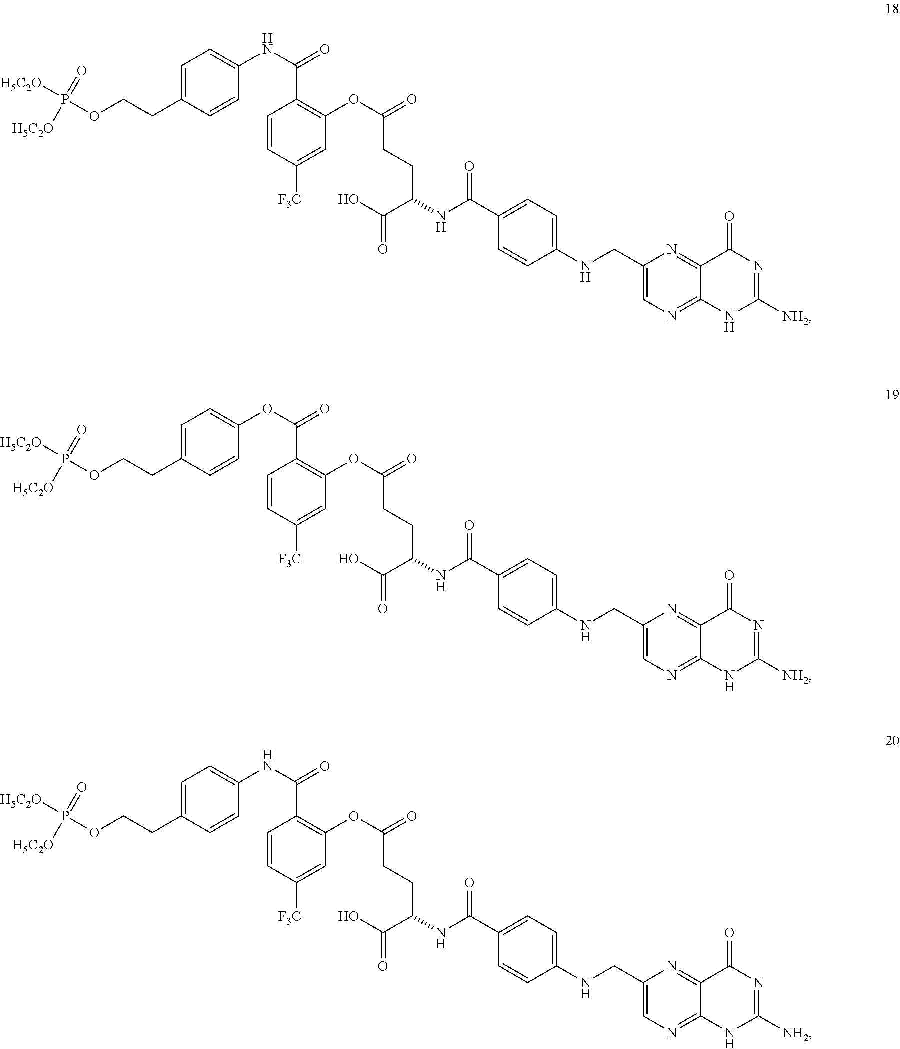

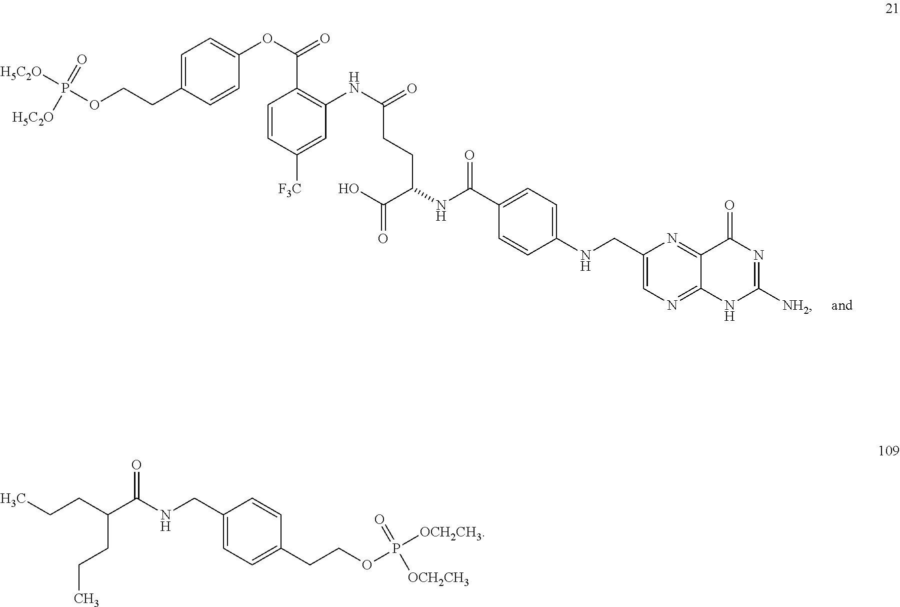

In a second aspect the invention features a compound of general Formula II

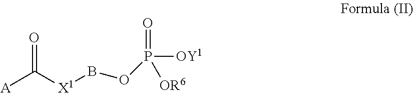

or a pharmaceutically acceptable salt thereof.

In Formula II: Y¹ is a polyethylene glycol residue;

R⁶ is selected from hydrogen, C₁₋₆-alkyl, and polyethylene glycol residue;

A is an optionally substituted aliphatic, heteroaliphatic, aromatic, heteroaromatic substituent or alkylaryl substituent having 1 to 100 carbon atoms or selected from:

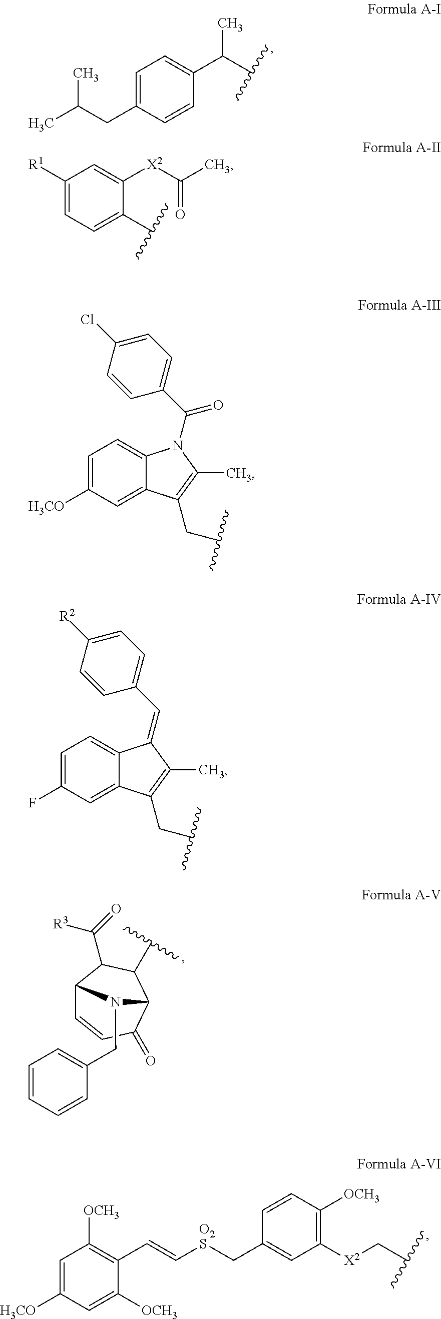

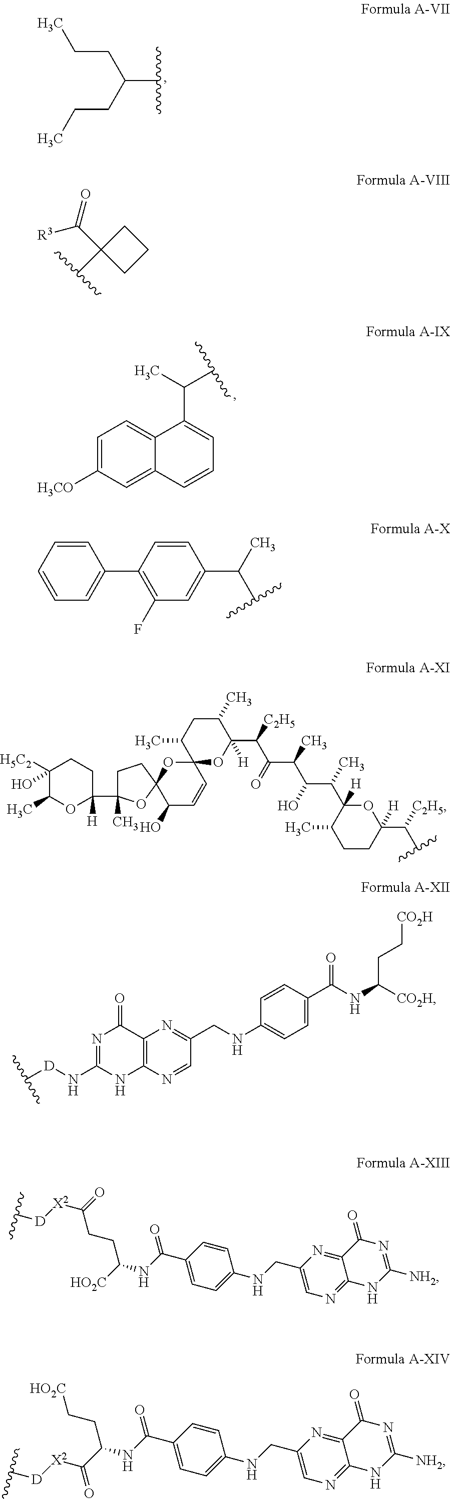

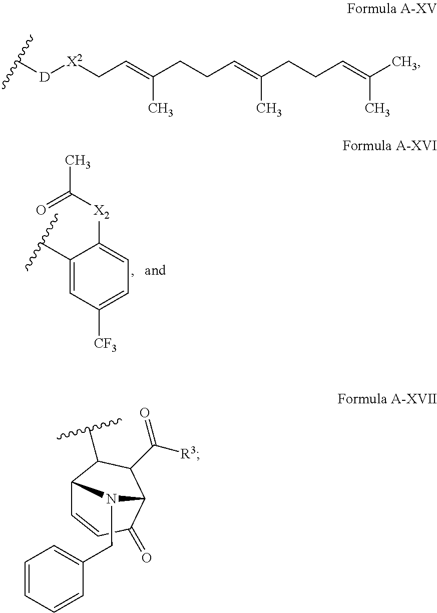

D is absent or

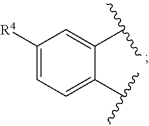

X¹ and X² are independently selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B-Z;

R⁵ is selected from hydrogen and C₁₋₆ alkyl;

B is selected from:

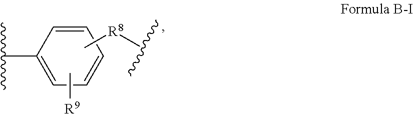

a single bond, and an aliphatic group with 1 to 22 carbon atoms;

R⁸ is a C₁₋₄ alkylene; and

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl.

In further embodiments, Y¹ is a polyethylene glycol residue described by —O(CH₂CH₂O)ₘR¹⁰, wherein m is 1 to 100 (e.g. 20 to 100, 20 to 50, 40 to 50), and R¹⁰ is selected from hydrogen, alkyl and alkoxy, and R⁶ is hydrogen.

In still other embodiments, Y¹ is —O(CH₂CH₂O)ₘR¹⁰ wherein m is 45, R¹⁰ is —OCH₃, and R⁶ is hydrogen.

In some embodiments, X¹ is —O—.

In other embodiments, X¹ is —NR⁵— and R⁵ is selected from hydrogen, methyl, and ethyl.

In certain embodiments, B is —(CH₂)₄—.

In some embodiments, A is:

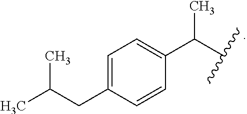 [SMILES: *C(C)c1ccc(CC(C)C)cc1]

In other embodiments, the compound is:

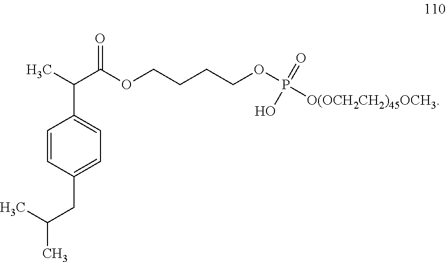

In a third aspect, the invention features a compound of general Formula III

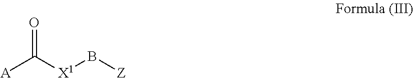

or a pharmaceutically acceptable salt thereof.

In Formula III: A is selected from:

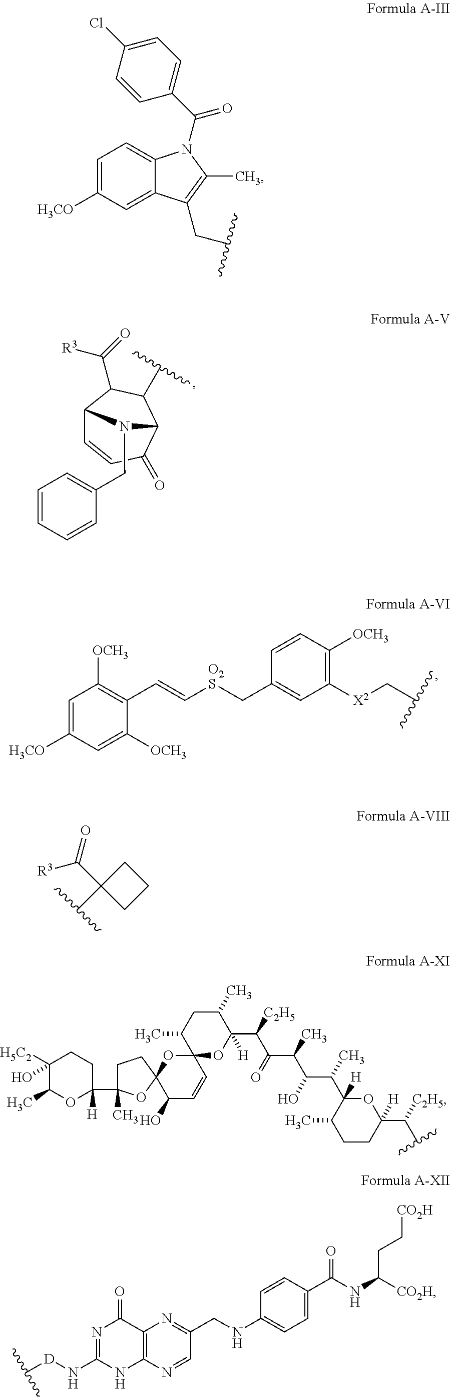

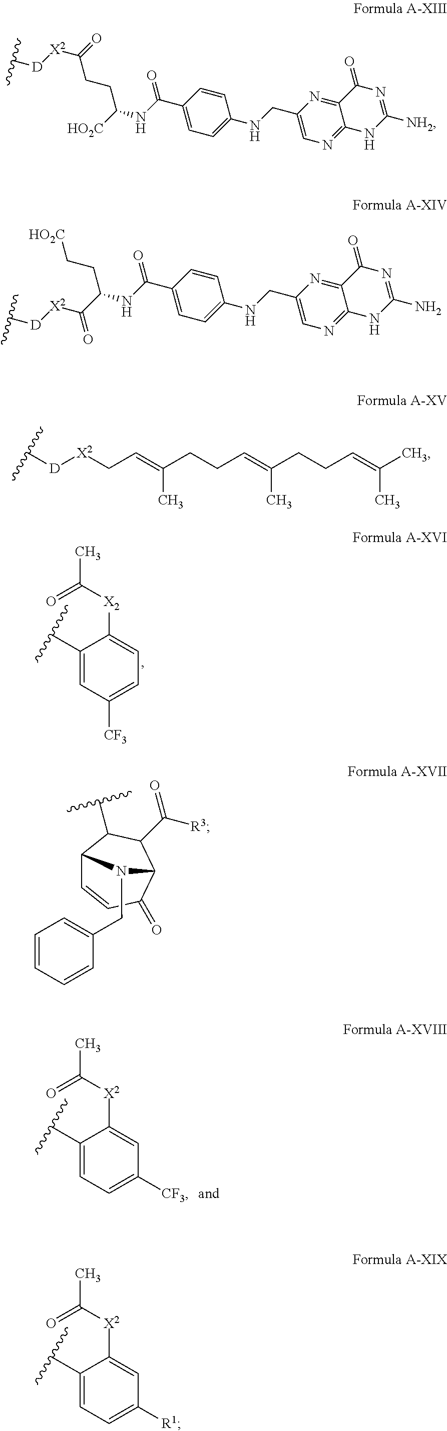

D is absent or

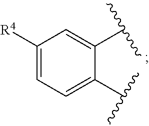

X¹ and X² are independently selected from —O—, —NR⁵—, and —S—;

R¹ and R⁴ are independently selected from hydrogen and trifluoromethyl;

X³ is selected from —S— and —NH—;

R³ is selected from hydroxyl, Z, and —X¹—B-Z;

R⁵ is selected from hydrogen and C₁₋₆ alkyl;

B is selected from:

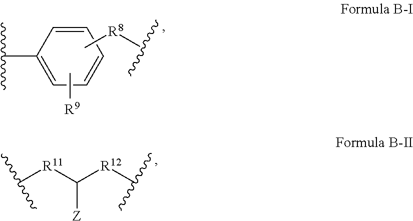

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;

Z is selected from:

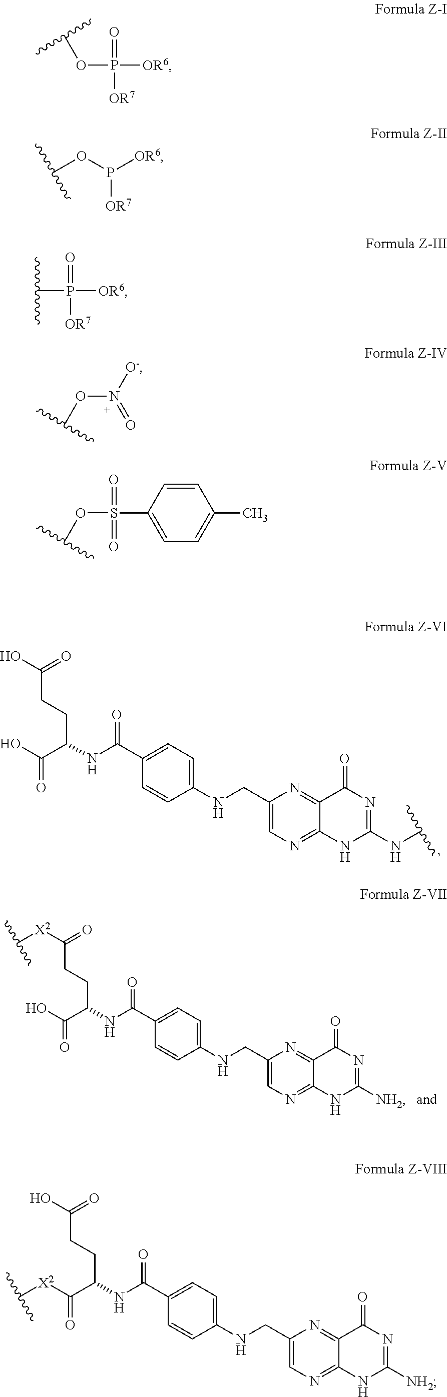

or B together with Z forms a structure:

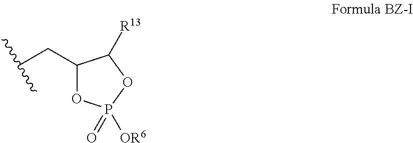

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

In still other embodiments, X¹ is —O—.

In certain embodiments, X¹ is —NR⁵— and R⁵ is selected from hydrogen, methyl, and ethyl.

In some embodiments, B is selected from:

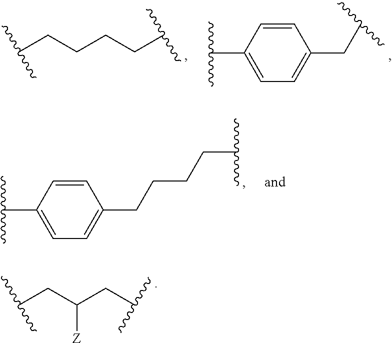

In other embodiments, Z is selected from —OP(O)(OCH₂CH₃)₂ and —ONO₂.

In further embodiments, BZ is

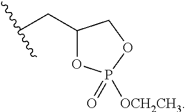 [SMILES: *CC1COP(=O)(OCC)O1]

In certain embodiments, X¹ is selected from —O— and —NH—, B is selected from

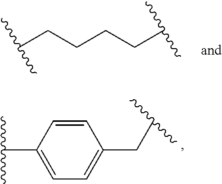 [SMILES: *CCCC* ; *Cc1ccc(*)cc1]

### Z is —OP(O)(OCH₂CH₃)₂, and A is:

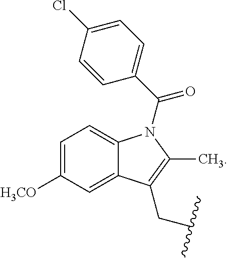 [SMILES: *Cc1c(C)n(C(=O)c2ccc(Cl)cc2)c2ccc(OC)cc12]

In some embodiments, X¹ is selected from —O— and —NH—, B is selected from

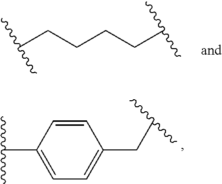 [SMILES: *CCCC* ; *Cc1ccc(*)cc1]

### Z is —OP(O)(OCH₂CH₃)₂, A is

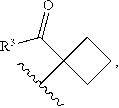

### and R³ is:

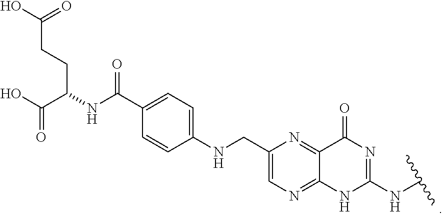 [SMILES: *Nc1nc(=O)c2nc(CNc3ccc(C(=O)N[C@@H](CCC(=O)O)C(=O)O)cc3)cnc2[nH]1]

In some embodiments, wherein X¹ is selected from —O— and —NH—, B is selected from

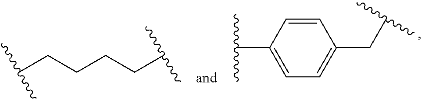 [SMILES: *CCCC* ; *Cc1ccc(*)cc1]

### Z is —OP(O)(OCH₂CH₃)₂, A is:

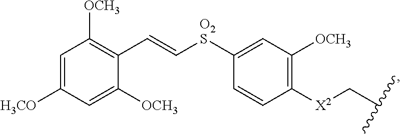

and X² is selected from —O— and —NH—.

In other embodiments, X¹ is selected from —O— and —NH—, B is selected from

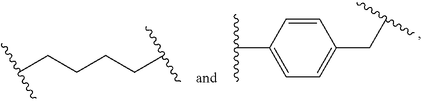 [SMILES: *CCCC* ; *Cc1ccc(*)cc1]

### Z is —OP(O)(OCH₂CH₃)₂, and A is:

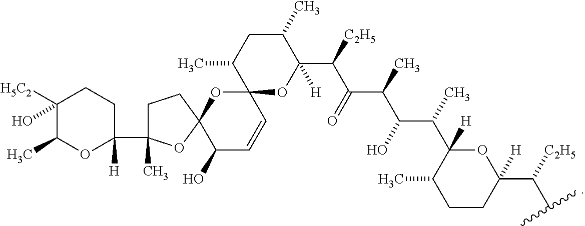

In further embodiments, X¹ is selected from —O— and —NH—, B is selected from

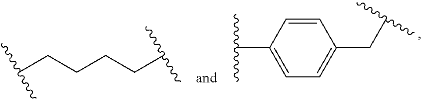 [SMILES: *CCCC* ; *Cc1ccc(*)cc1]

### Z is —OP(O)(OCH₂CH₃)₂, A is:

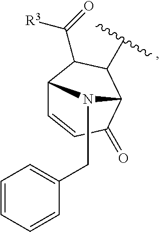

and R³ is hydroxyl or selected from:

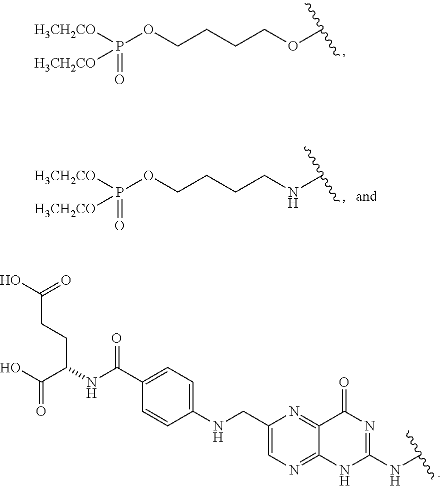

In certain embodiments, X¹ is selected from —O— and —NH—, B is selected from

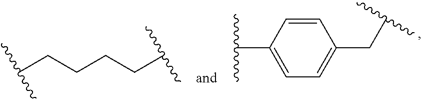 [SMILES: *CCCC* ; *Cc1ccc(*)cc1]

### Z is —OP(O)(OCH₂CH₃)₂, A is:

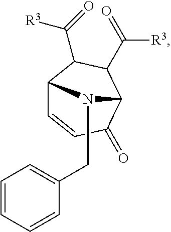

and R³ is hydroxyl or selected from:

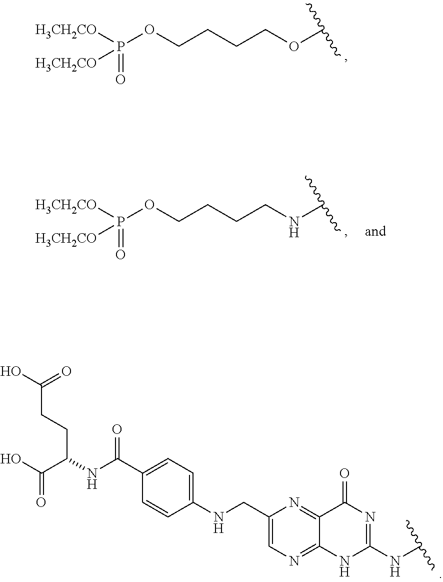

In some embodiments, X¹ is selected from —O— and —NH—, B is selected from

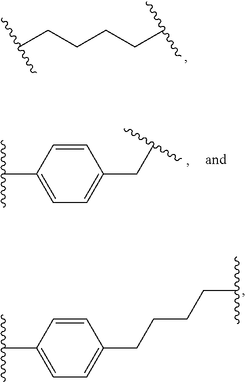 [SMILES: *CCCC* ; *Cc1ccc(*)cc1 ; *CCCCc1ccc(*)cc1]

### Z is —OP(O)(OCH₂CH₃)₂, A is:

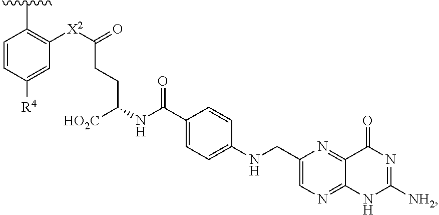

and R⁴ is selected from hydrogen and trifluoromethyl.

In some embodiments, X¹ is selected from —O— and —NH—, B is selected from

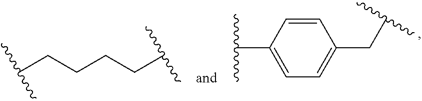 [SMILES: *CCCC* ; *Cc1ccc(*)cc1]

### Z is —OP(O)(OCH₂CH₃)₂, A is:

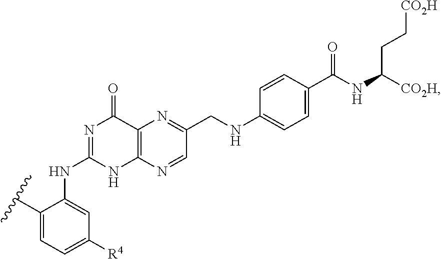

and R⁴ is selected from hydrogen and trifluoromethyl.

In other embodiments, X¹ is selected from —O— and —NH—, B is selected from

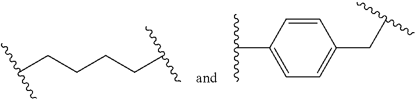 [SMILES: *CCCC* ; *Cc1ccc(*)cc1]

### Z is —OP(O)(OCH₂CH₃)₂, A is:

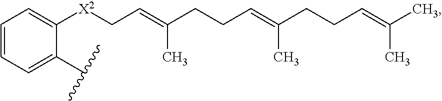

and X² is selected from —O—, —S—, and —NH—.

In other embodiments, X¹ is selected from —O— and —NH—, B is selected from

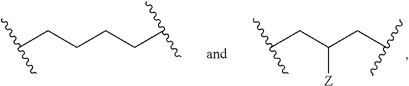

Z is selected from —OP(O)(OCH₂CH₃)₂ and —ONO₂, A is:

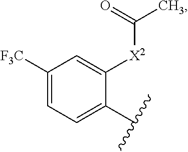

and X² is selected from —O—, —S—, and —NH—.

In some embodiments, X¹ is selected from —O— and —NH—, B is —(CH₂)₄—, Z is —ONO₂, A is:

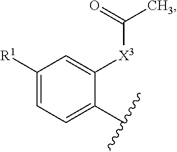

R¹ is selected from hydrogen and trifluoromethyl, and X³ is selected from —S—, and —NH—.

In other embodiments, X¹ is —NH—, A is:

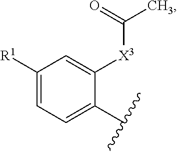

R¹ is selected from hydrogen and trifluoromethyl, and X³ is selected from —S—, and —NH—.

Accordingly, the compounds of Formula III include but are not limited to compounds of which the structures are shown below:

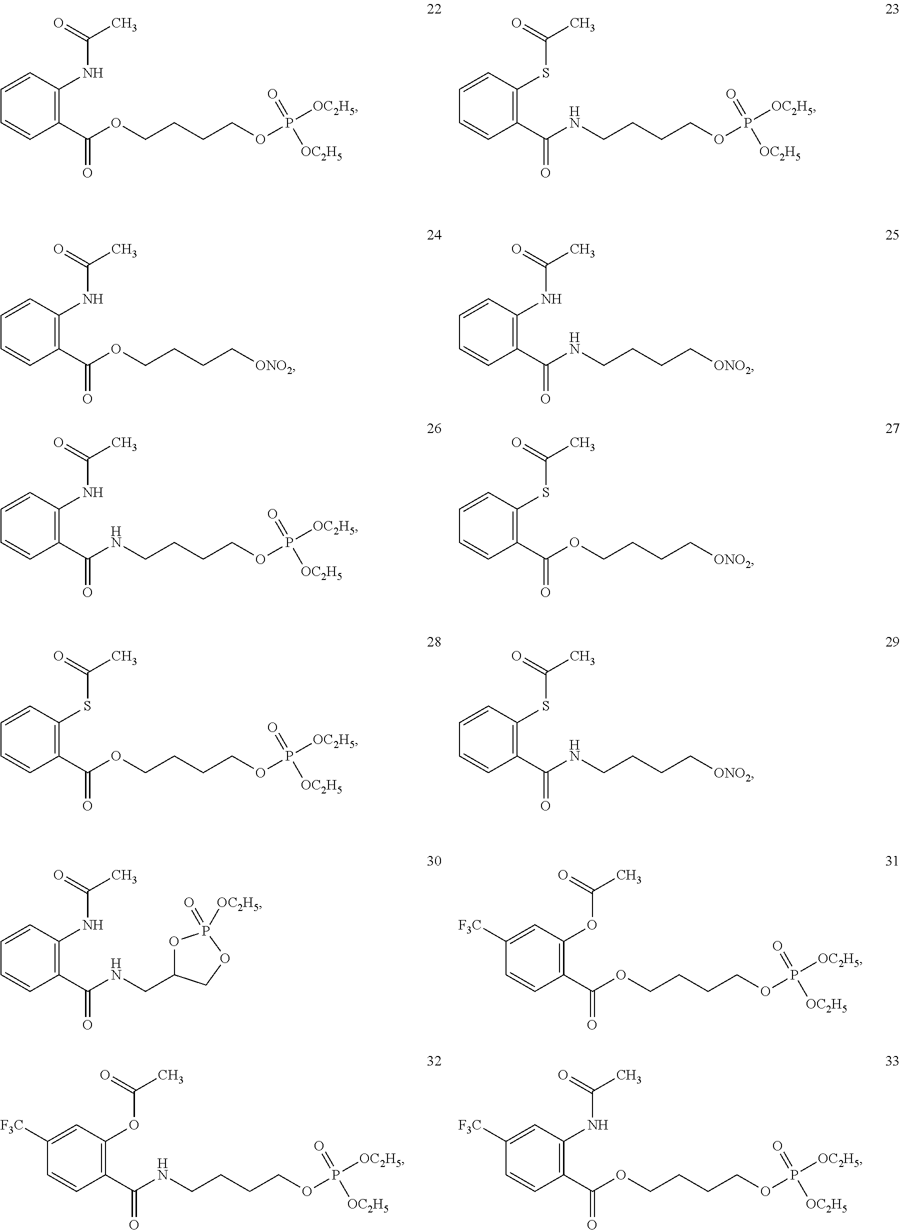

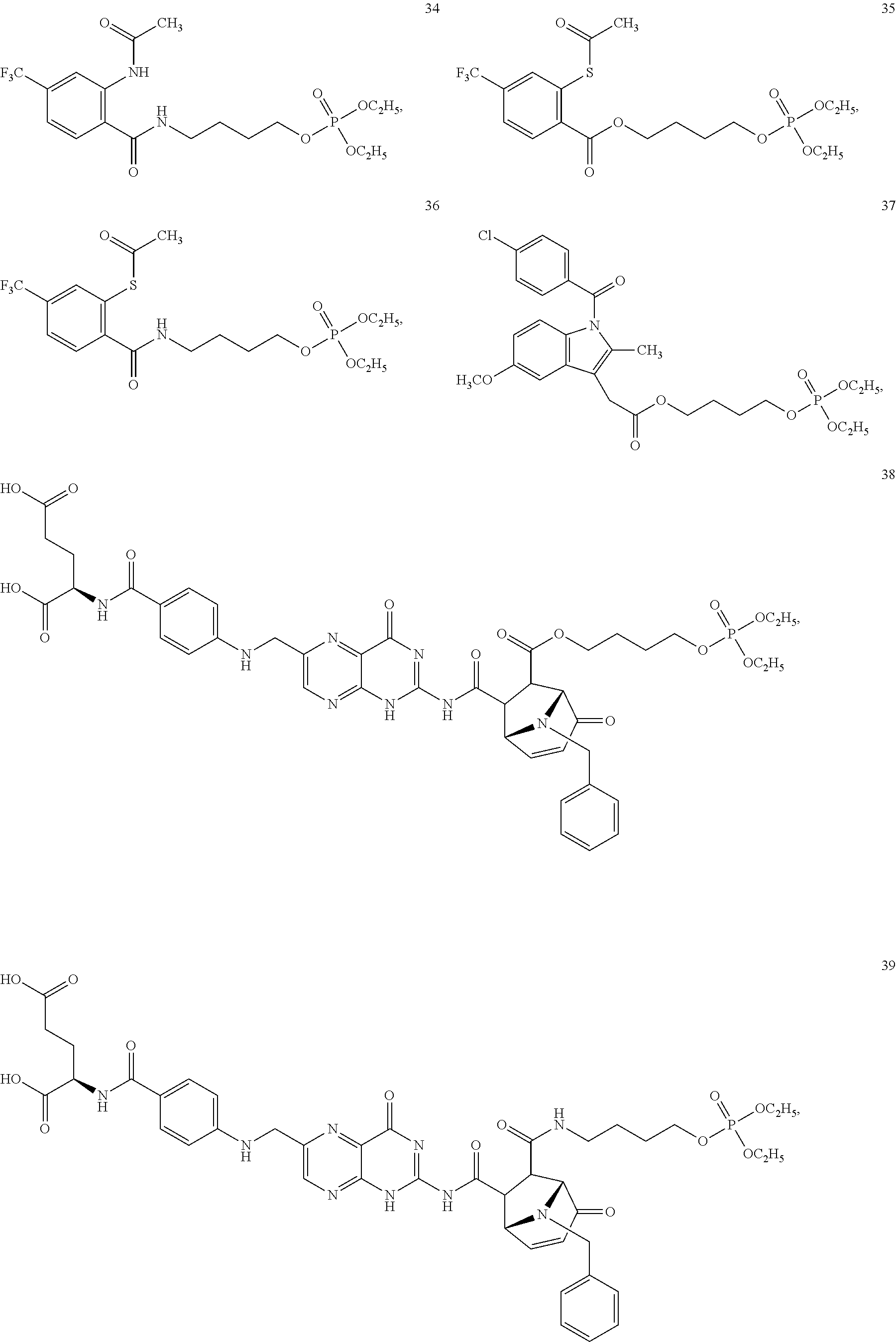 [SMILES: CCOP(=O)(OCC)OCCCCNC(=O)c1ccc(C(F)(F)F)cc1NC(C)=O ; CCOP(=O)(OCC)OCCCCOC(=O)c1ccc(C(F)(F)F)cc1SC(C)=O ; CCOP(=O)(OCC)OCCCCNC(=O)c1ccc(C(F)(F)F)cc1SC(C)=O ; CCOP(=O)(OCC)OCCCCOC(=O)Cc1c(C)n(C(=O)c2ccc(Cl)cc2)c2ccc(OC)cc12 ; CCOP(=O)(OCC)OCCCCOC(=O)C1C(C(=O)Nc2nc(=O)c3nc(CNc4ccc(C(=O)N[C@H](CCC(=O)O)C(=O)O)cc4)cnc3[nH]2)[C@H]2C=CC(=O)[C@@H]1N2Cc1ccccc1 ; CCOP(=O)(OCC)OCCCCNC(=O)C1C(C(=O)Nc2nc(=O)c3nc(CNc4ccc(C(=O)N[C@H](CCC(=O)O)C(=O)O)cc4)cnc3[nH]2)[C@H]2C=CC(=O)[C@@H]1N2Cc1ccccc1]

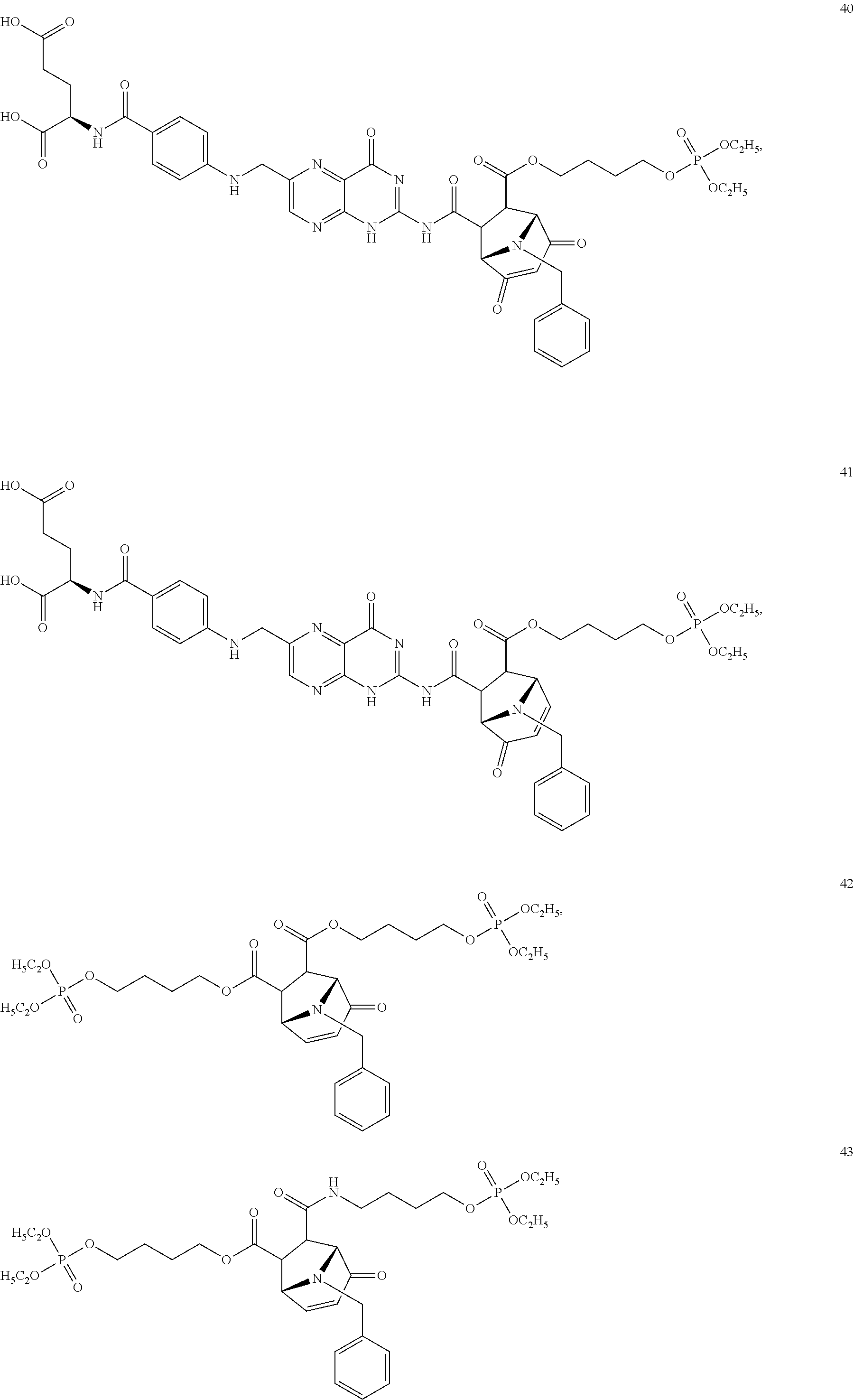

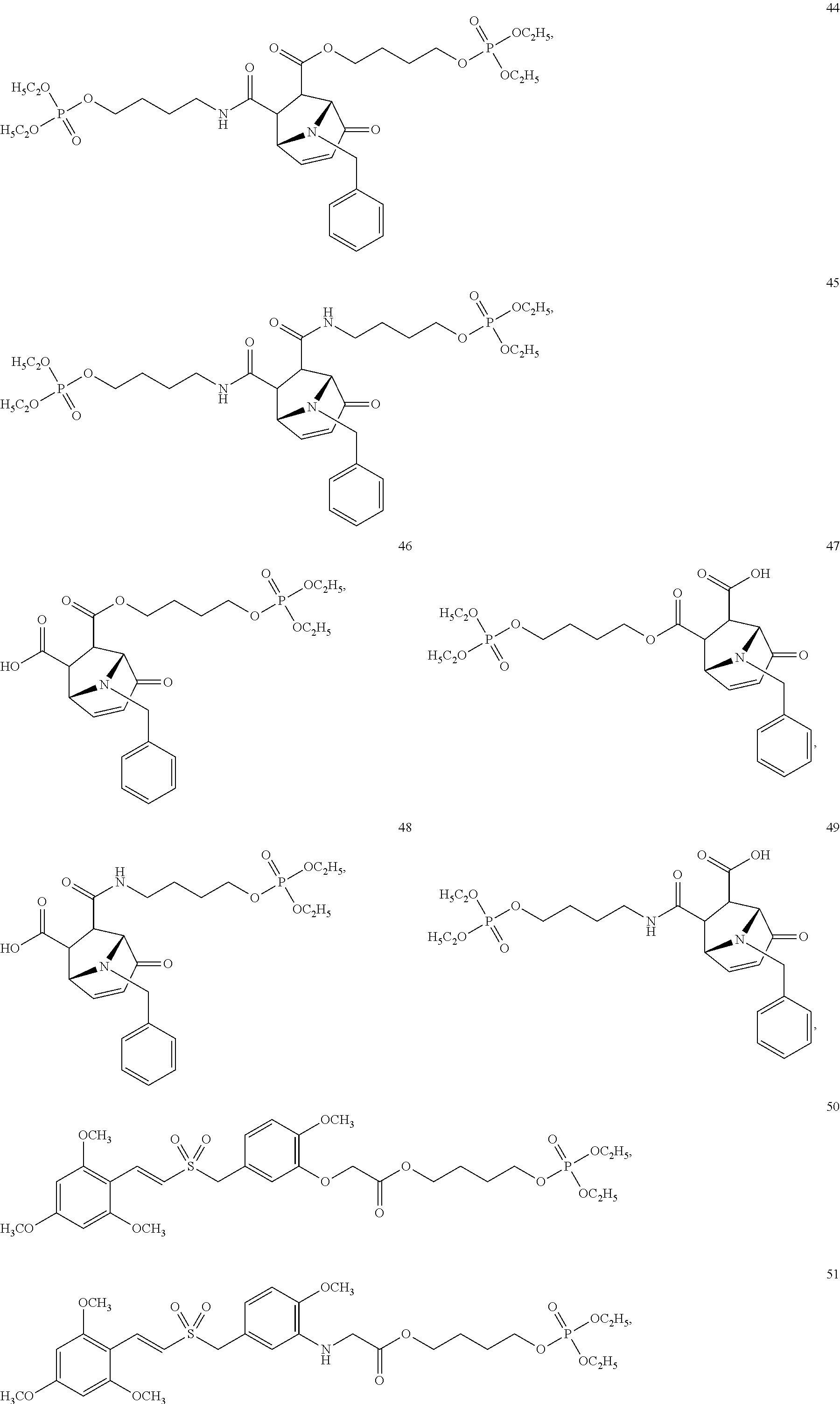

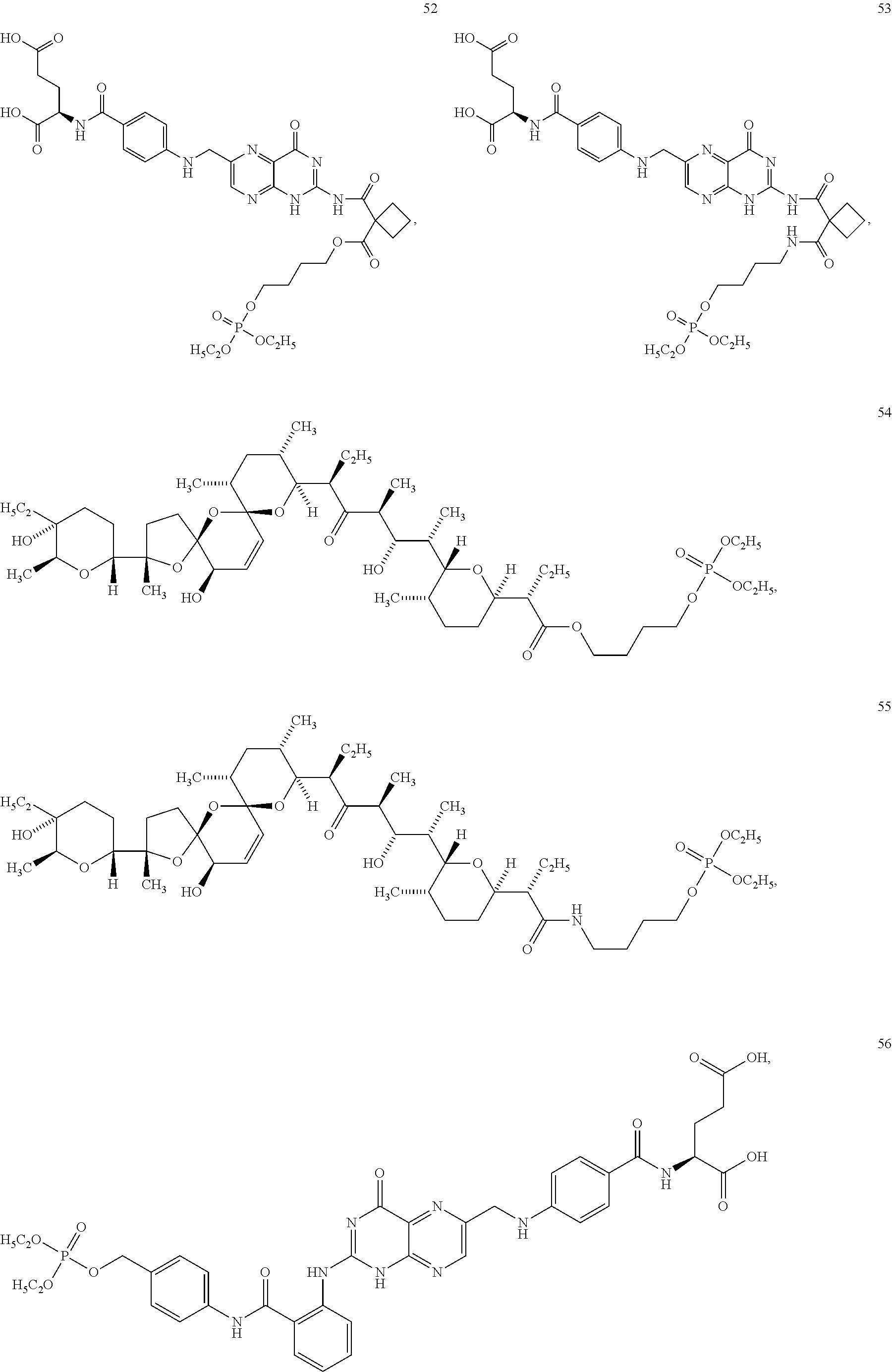

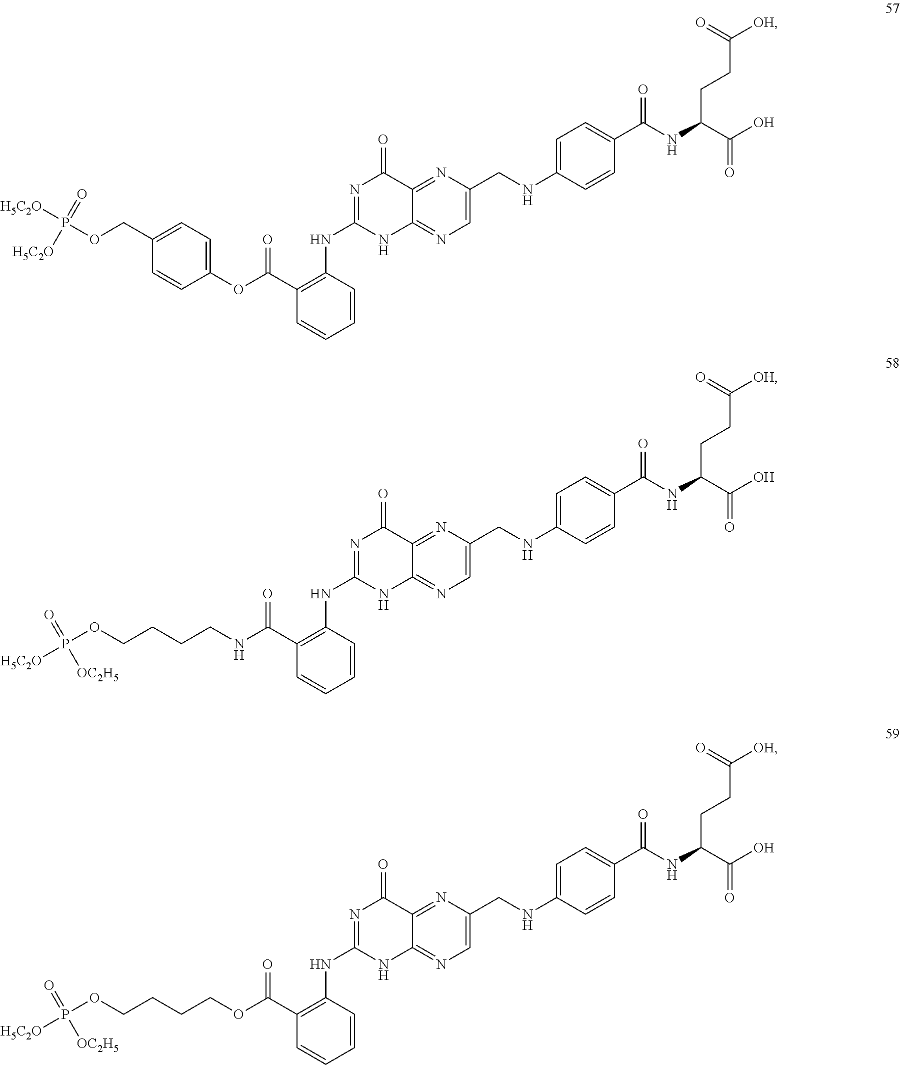

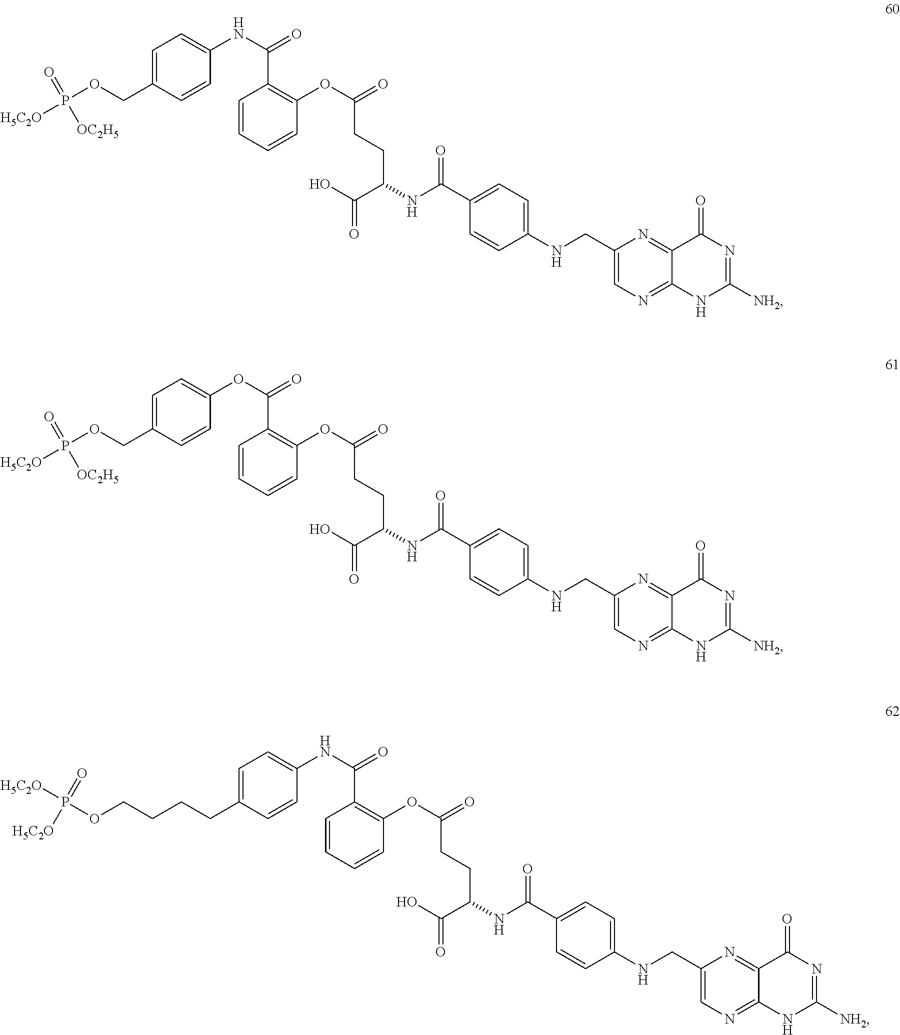

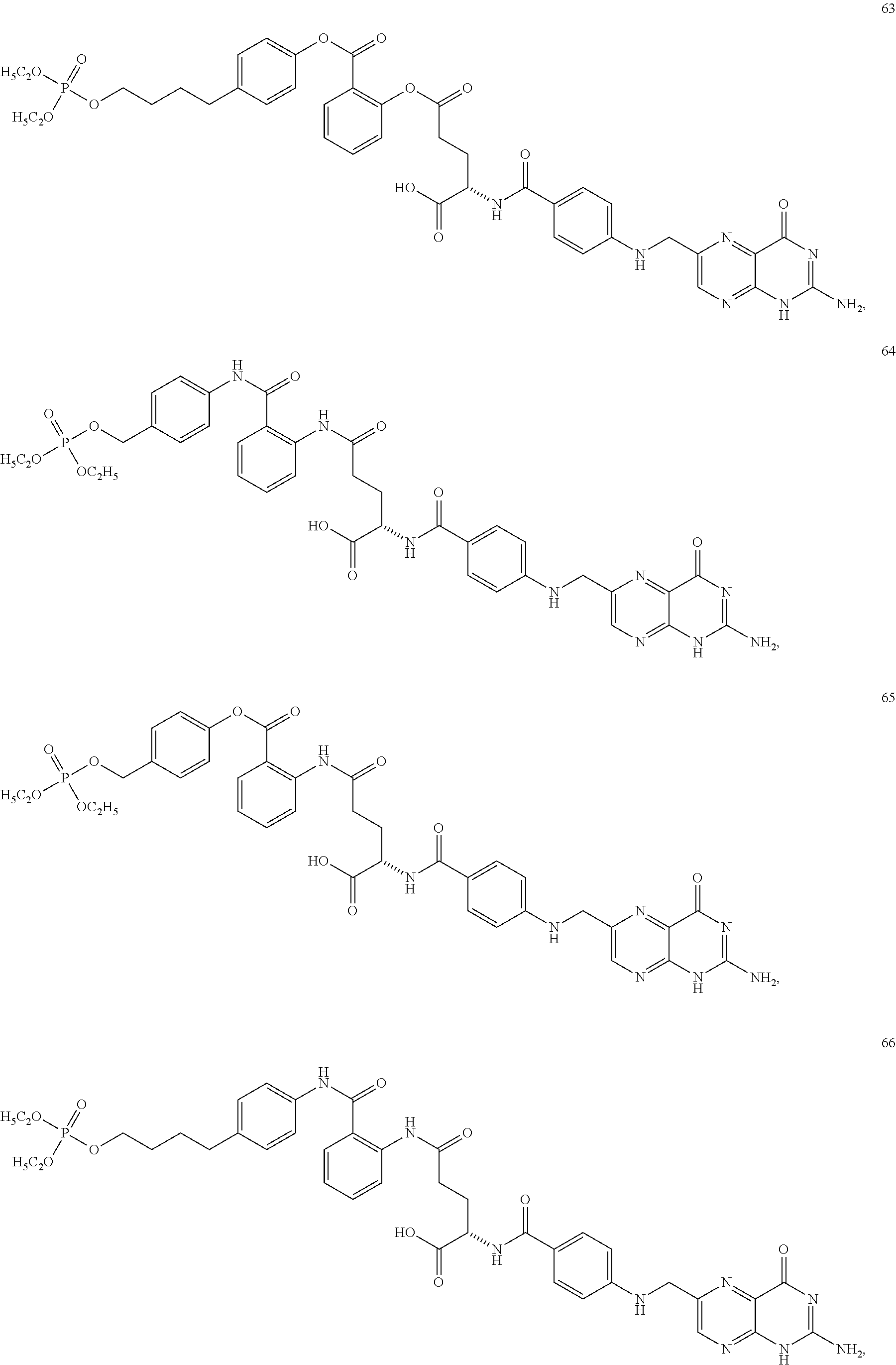

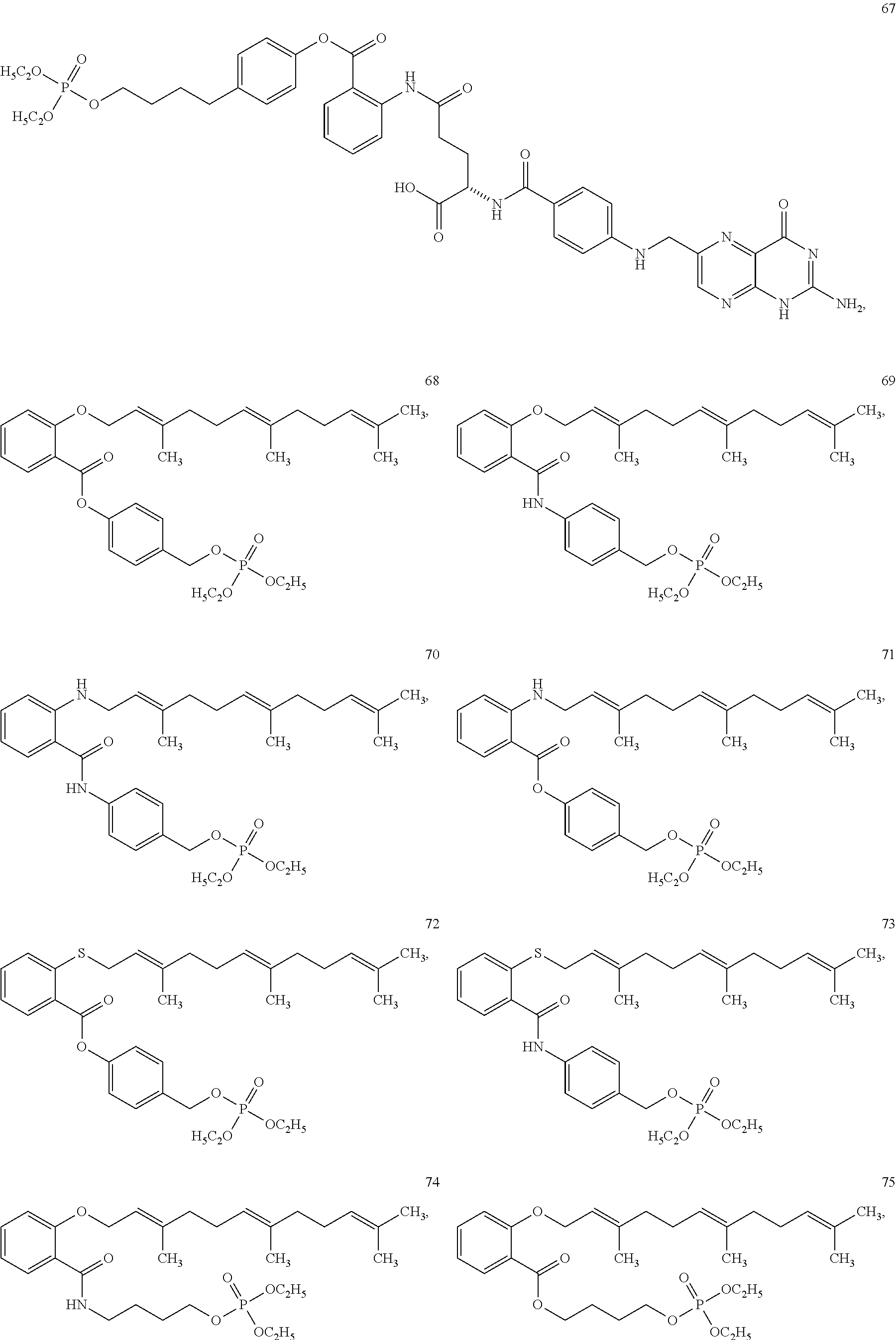

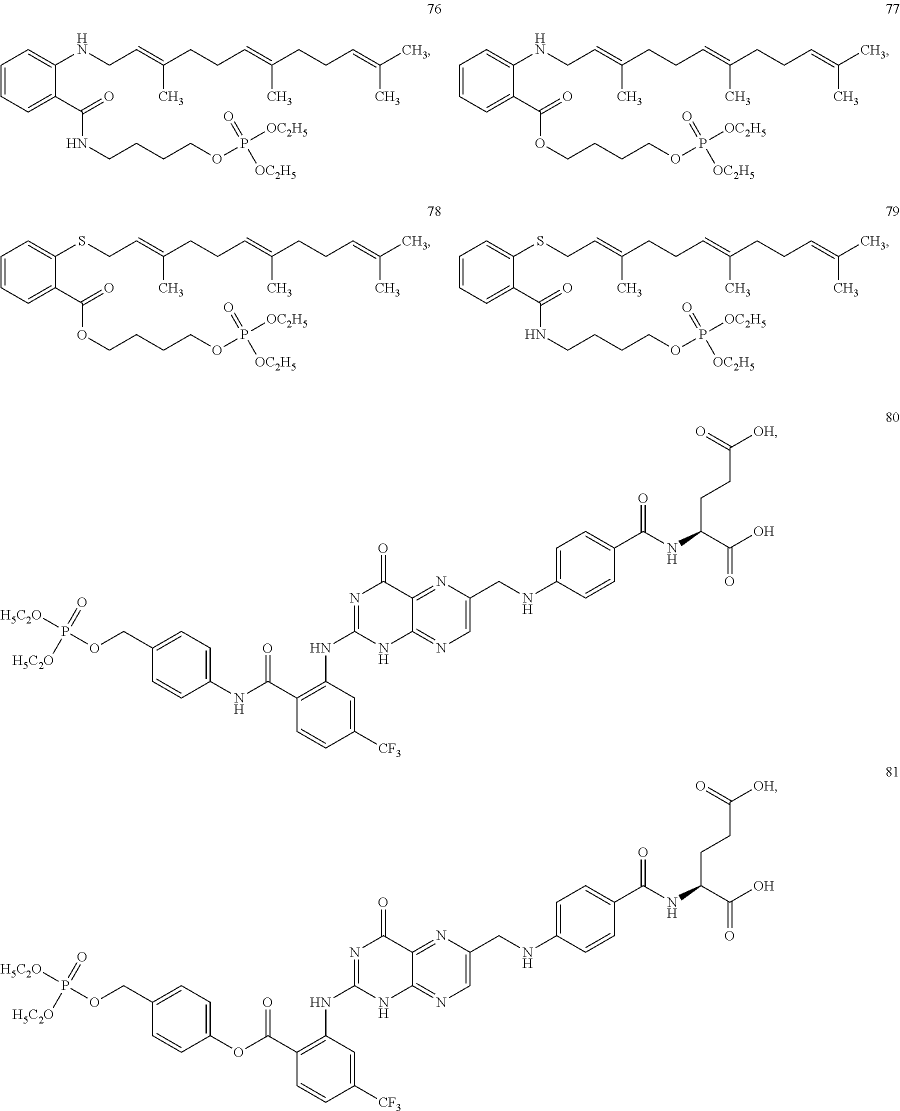

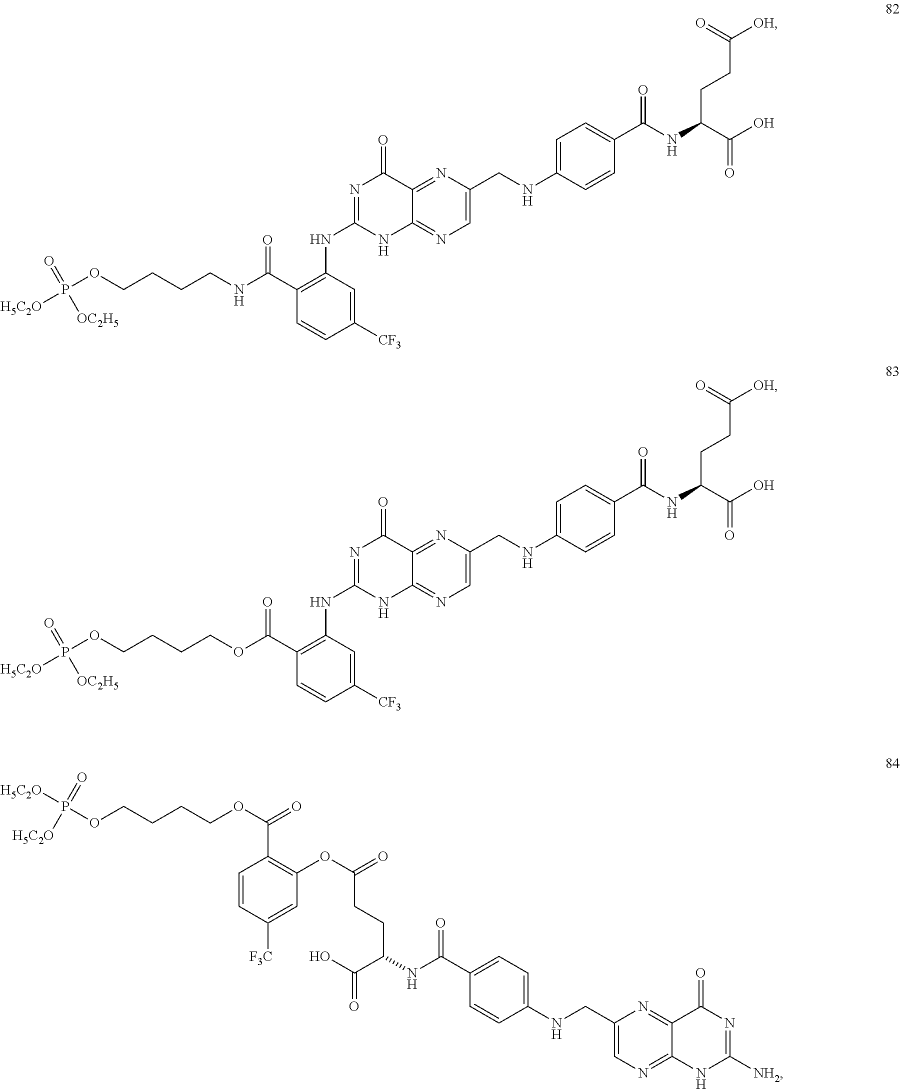

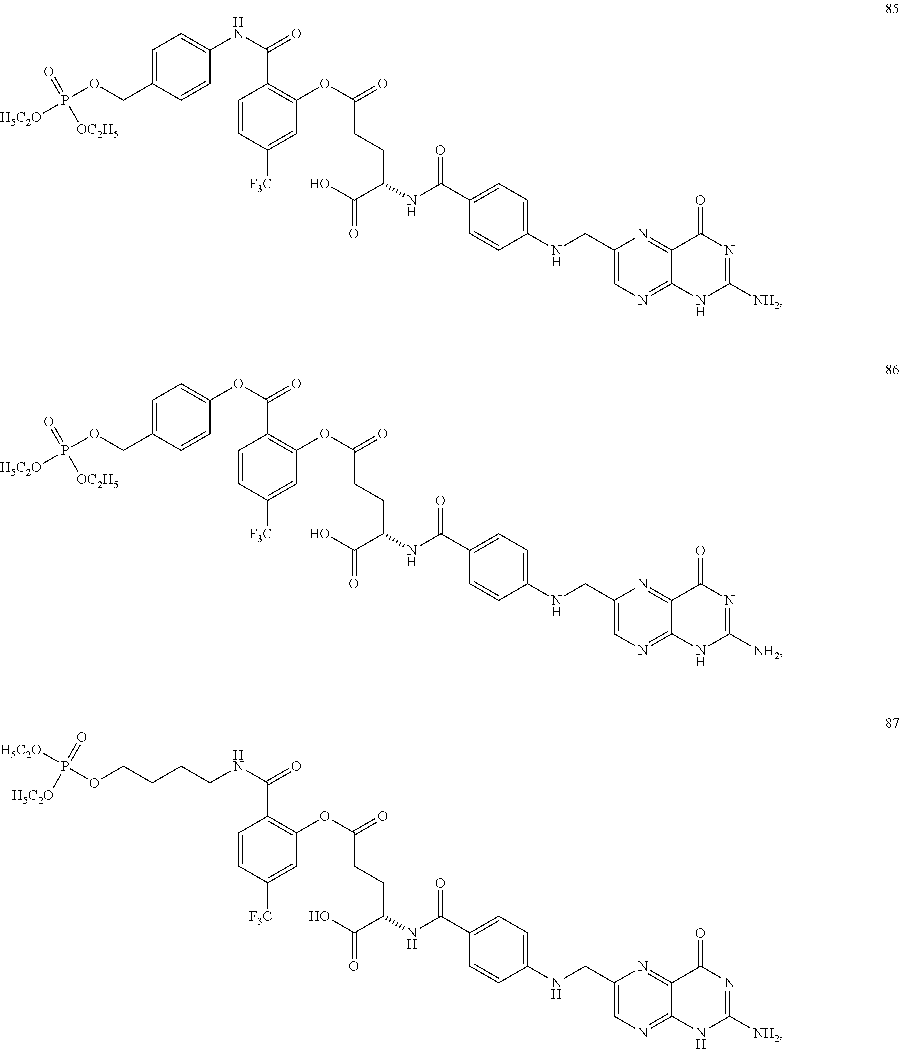

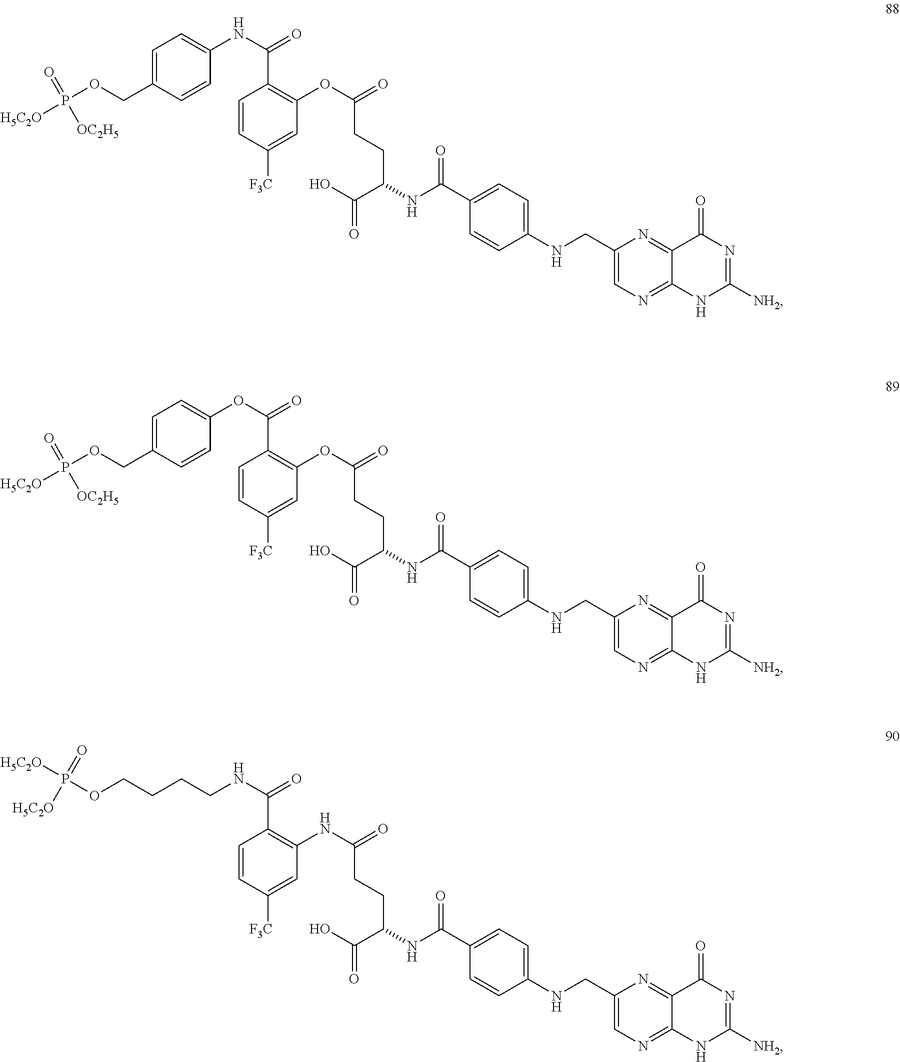

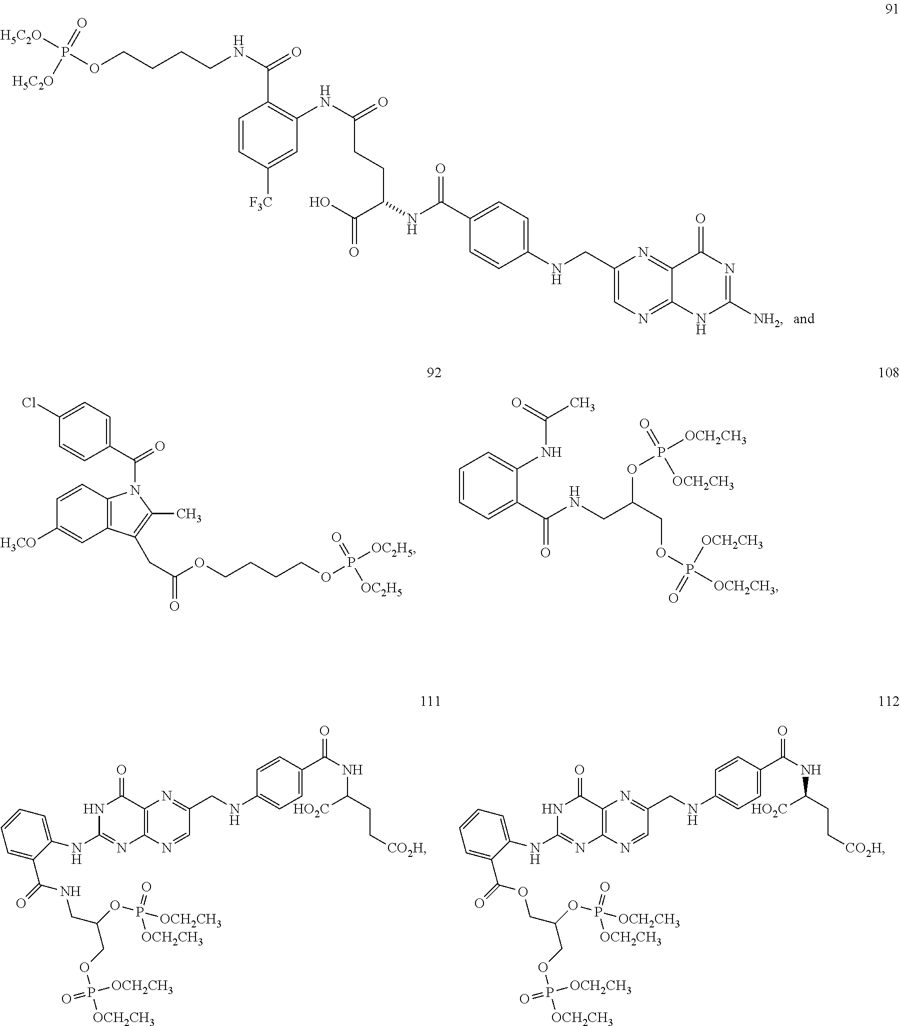

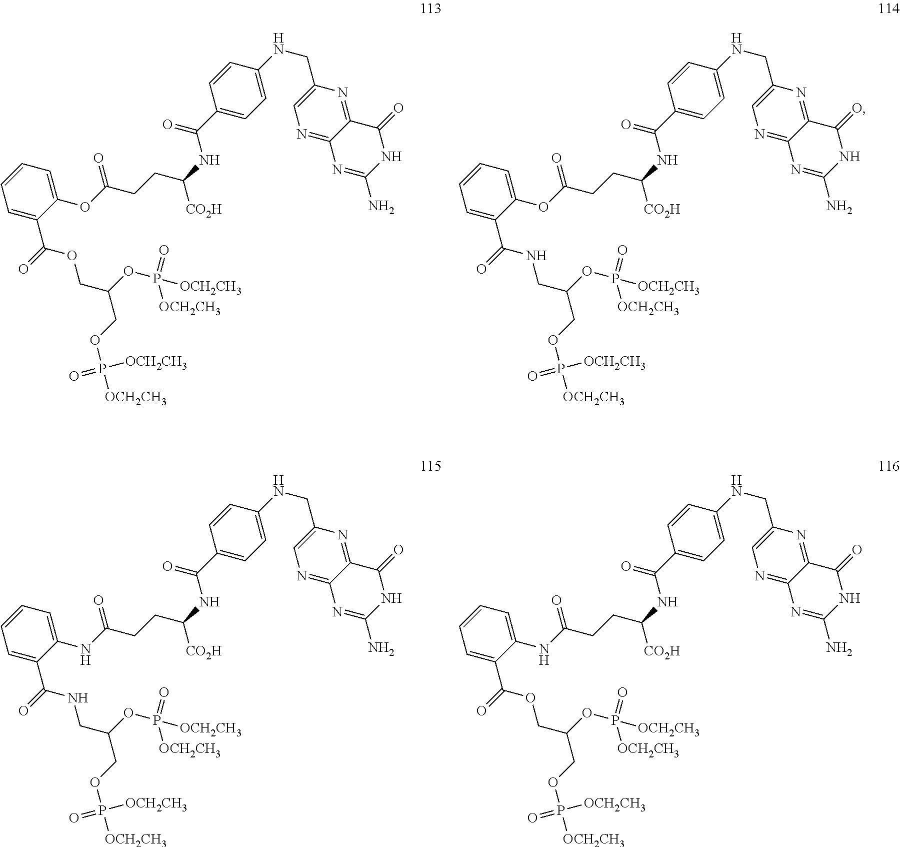 [SMILES: CCOP(=O)(OCC)OCC(COC(=O)c1ccccc1OC(=O)CC[C@@H](NC(=O)c1ccc(NCc2cnc3nc(N)[nH]c(=O)c3n2)cc1)C(=O)O)OP(=O)(OCC)OCC ; CCOP(=O)(OCC)OCC(CNC(=O)c1ccccc1OC(=O)CC[C@@H](NC(=O)c1ccc(NCc2cnc3nc(N)[nH]c(=O)c3n2)cc1)C(=O)O)OP(=O)(OCC)OCC ; CCOP(=O)(OCC)OCC(CNC(=O)c1ccccc1NC(=O)CC[C@@H](NC(=O)c1ccc(NCc2cnc3nc(N)[nH]c(=O)c3n2)cc1)C(=O)O)OP(=O)(OCC)OCC ; CCOP(=O)(OCC)OCC(COC(=O)c1ccccc1NC(=O)CC[C@@H](NC(=O)c1ccc(NCc2cnc3nc(N)[nH]c(=O)c3n2)cc1)C(=O)O)OP(=O)(OCC)OCC]

In a fourth aspect the invention features a compound of general Formula IV

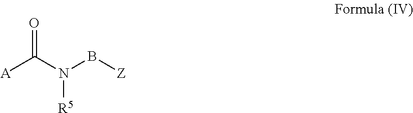

or a pharmaceutically acceptable salt thereof.

In Formula IV: A is an optionally substituted aliphatic, heteroaliphatic, aromatic, heteroaromatic substituent or alkylaryl substituent having 1 to 100 carbon atoms or selected from:

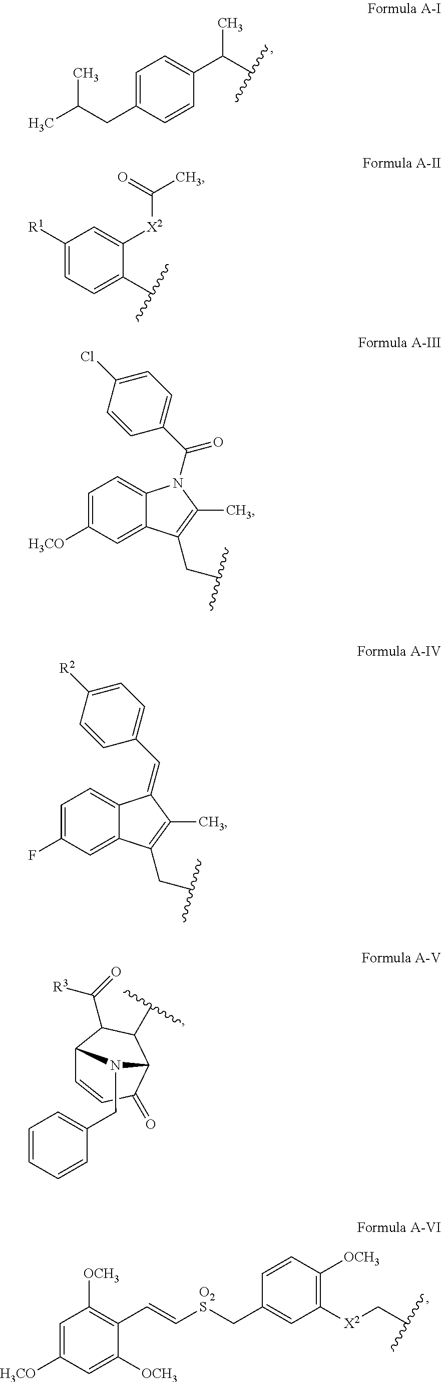

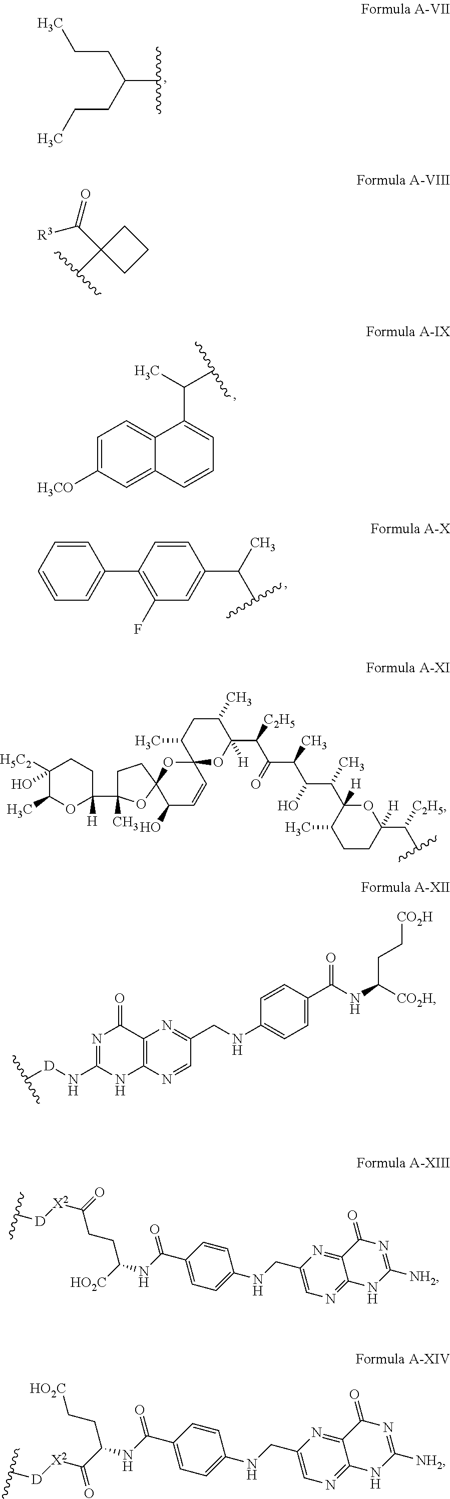

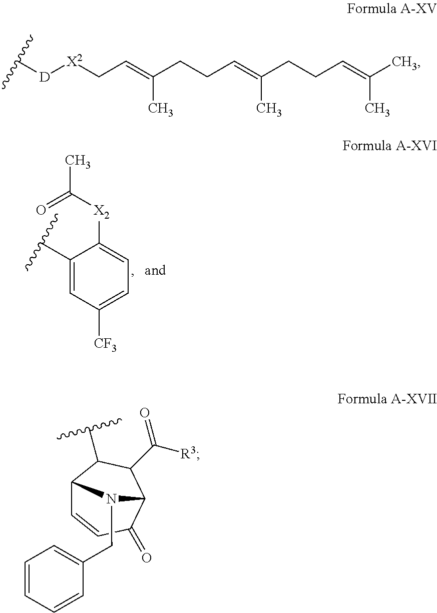

D is absent or

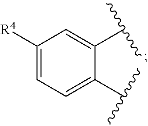

X² is selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B-Z;

R⁵ is selected from methyl and ethyl;

B is selected from:

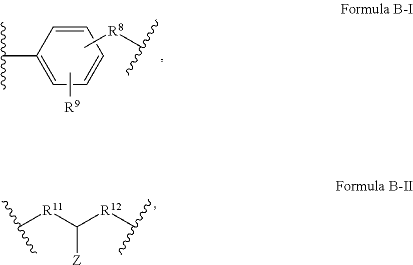

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;

Z is selected from:

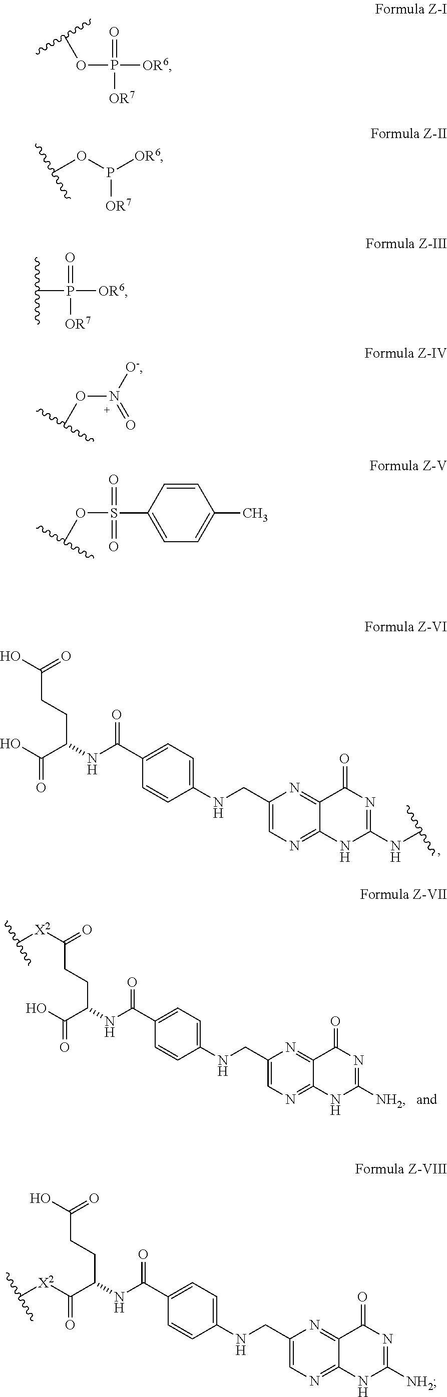

or B together with Z forms a structure:

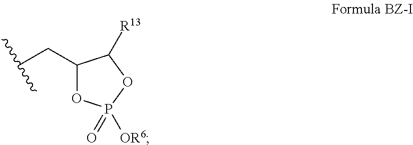

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

In a fifth aspect, the invention features a compound having a structure selected from the group consisting of

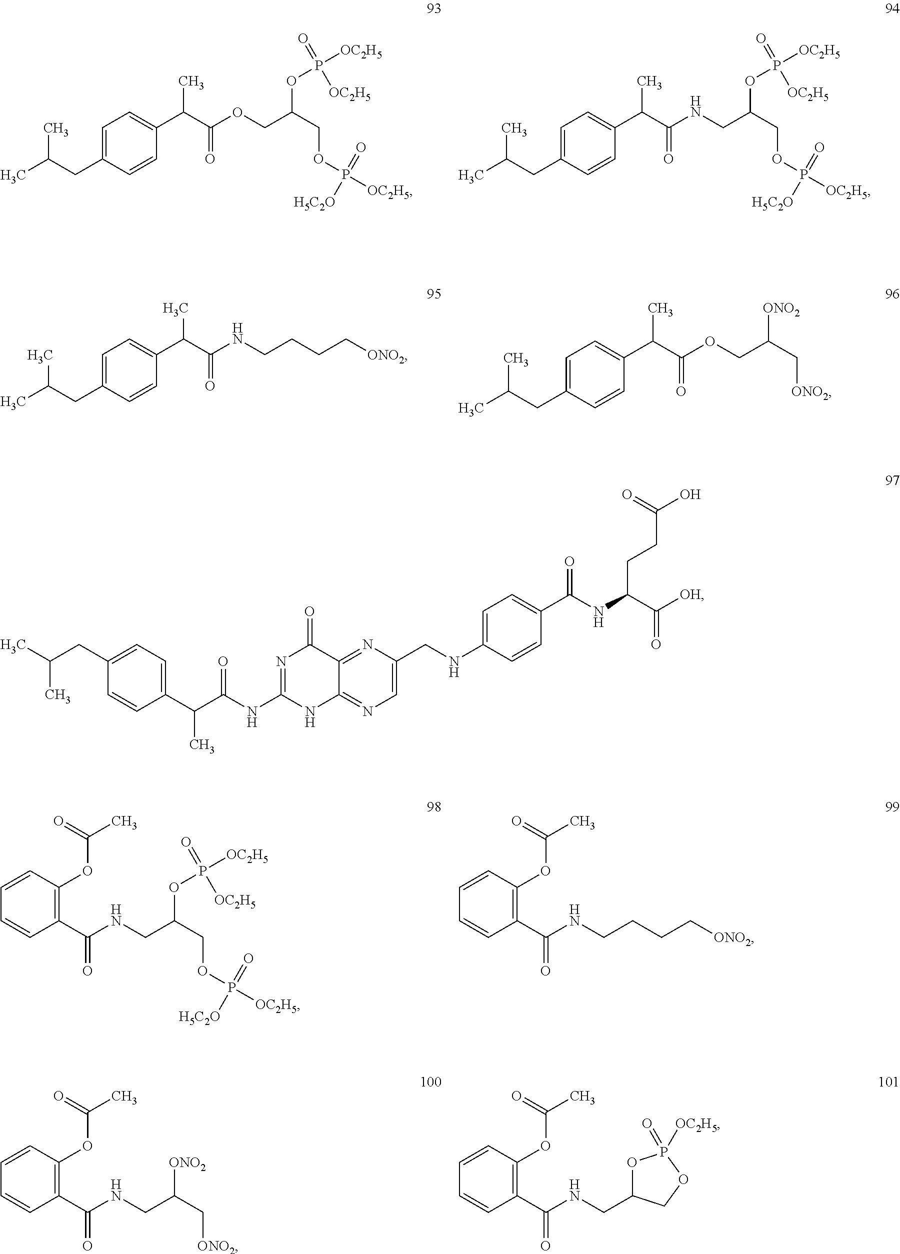

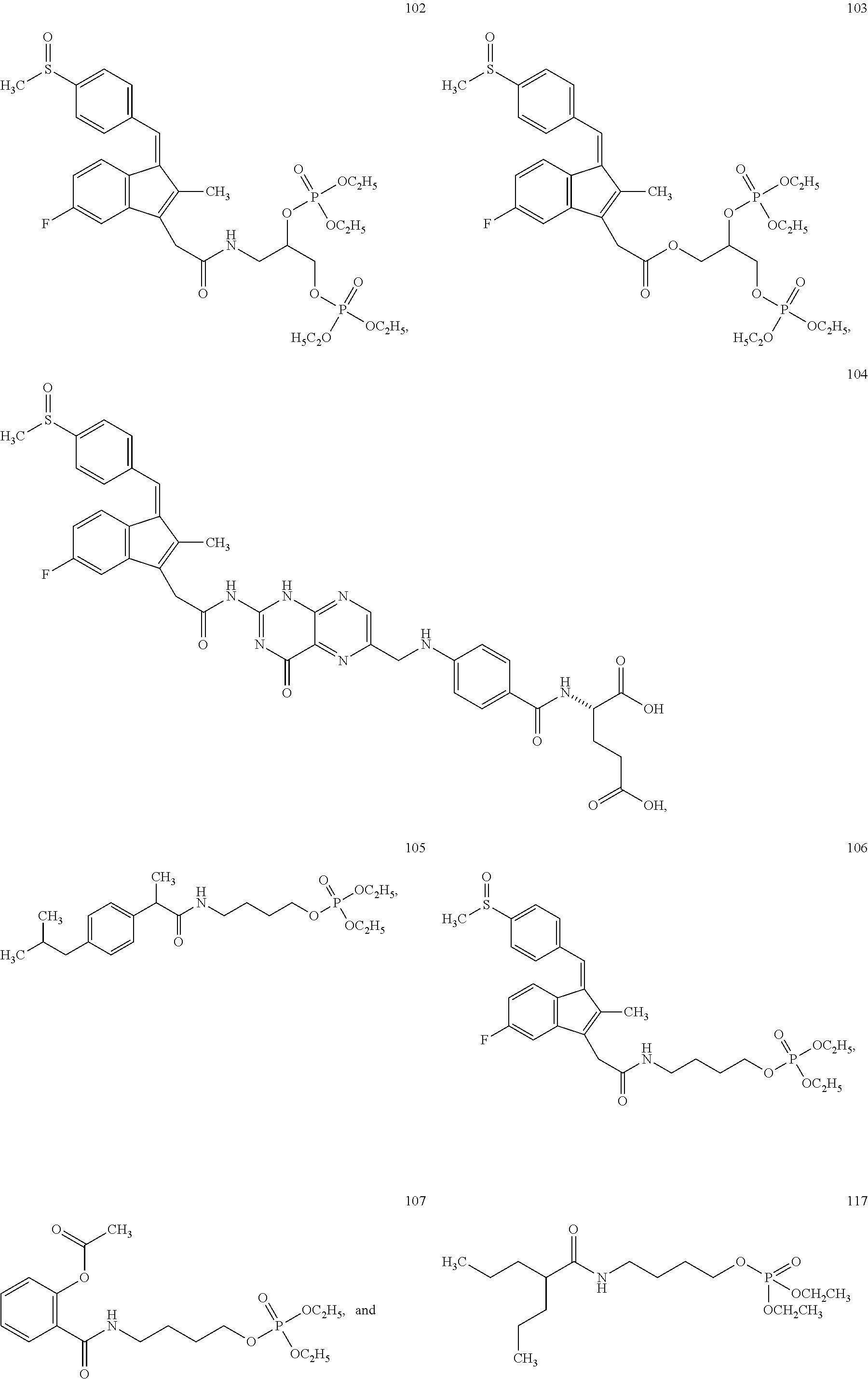

A further aspect of the present invention is directed to a topical pharmaceutical composition comprising a compound of one of Formulas I-IV or any compound specified above, as described generally herein, and a pharmaceutically acceptable excipient.

In a specific embodiment, the composition further comprises difluoromethylornithine or cimetidine.

Another aspect of the present invention relates to the use of an effective amount of compounds represented by Formulas I-IV, any compound specified above or any composition described herein in the treatment of inflammation of a subject in need thereof.

In a specific embodiment, the compound is useful in the treatment of inflammation related to rheumatoid arthritis, Sjogren's syndrome, coronary artery disease, peripheral vascular disease, hypertension, Alzheimer's disease and its variants, lupus erythematosus, chronic bronchitis, chronic sinusitis, benign prostatichypertrophy, prostate cancer, colon adenomas, colon cancer, cancer of the lung, lymphoma, and leukemia.

A further aspect of the present invention relates to the use of an effective amount of compounds represented by Formula I, II, III, or IV, or any specific compound or composition described herein for the treatment or prevention of cancer in a subject in need thereof.

In yet another aspect, the present invention features methods for treating cell proliferation by contacting a cell with an effective amount of a compound represented by Formula I, II, III, or IV, or any specific compound or composition described herein.

In a further aspect, the present invention features methods for treating non-cancerous conditions of the skin or mucous membranes, the method including topically administering to a subject in need thereof an effective amount of a compound of Formula V

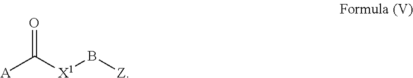

In Formula V: A is an optionally substituted aliphatic, heteroaliphatic, aromatic, heteroaromatic substituent or alkylaryl substituent having 1 to 100 carbon atoms;

X¹ is selected from —O—, —S—, and —NR⁵—;

R⁵ is selected from hydrogen and a C₁₋₆ alkyl;

B is an aliphatic, alicyclic, heteroaliphatic, heterocyclic, aryl, aralkyl, or heteroaromatic group optionally substituted with one or more R¹⁵ moieties,

each R¹⁴ is independently, selected from hydrogen, halogen, hydroxyl, alkoxyl, —CN; an optionally substituted aliphatic, alicyclic, heteroaliphatic, heterocyclic, aryl, aralkyl, heteroaromatic moiety; —OR^{R}, —S(═O)ₙR^{d}, —NR^{b}R^{c}, —C(═O)R^{a} and —C(═O)OR^{a}; n is 0-2; R^{a}, for each occurrence, is independently selected from hydrogen and an optionally substituted aliphatic, alicyclic, heteroaliphatic, heterocyclic, aryl, aralkyl, or a heteroaromatic moiety; each of R^{b} and R^{c}, for each occurrence, is independently selected from hydrogen; hydroxyl, SO₂R^{d}, and aliphatic, alicyclic, heteroaliphatic, heterocyclic, aryl, aralkyl, heteroaromatic or an acyl moiety; R^{d}, for each occurrence, is independently selected from hydrogen, —N(R^{c})₂, aliphatic, aryl and heteroaryl, R^{c}, for each occurrence, is independently hydrogen or aliphatic; and R^{R} is an optionally substituted aliphatic, alicyclic, heteroaliphatic, heterocyclic, aryl, aralkyl, heteroaromatic or acyl moiety;

Z is selected from:

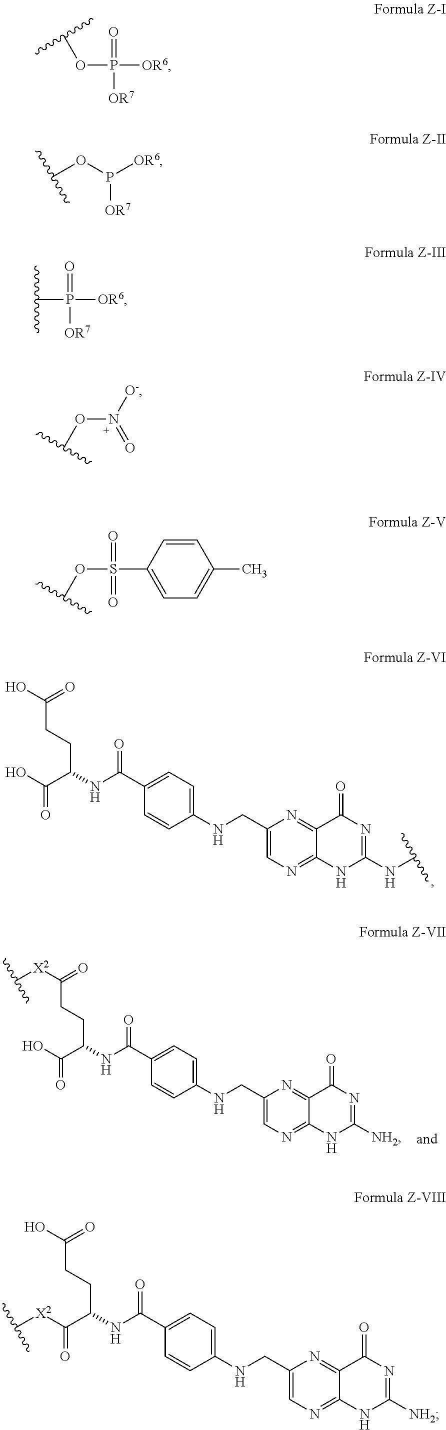

or B together with Z forms a structure:

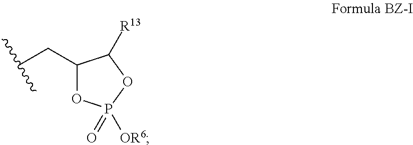

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue;

or a pharmaceutically acceptable salt thereof.

In a specific embodiment, the compound of Formula V is further described by Formula I, II, III, or IV or any specific compound described herein.

In another embodiment the compound of Formula V is a compound disclosed in U.S. Pat. No. 8,236,820, incorporated by reference. For example, the compound of Formula V can be selected from:

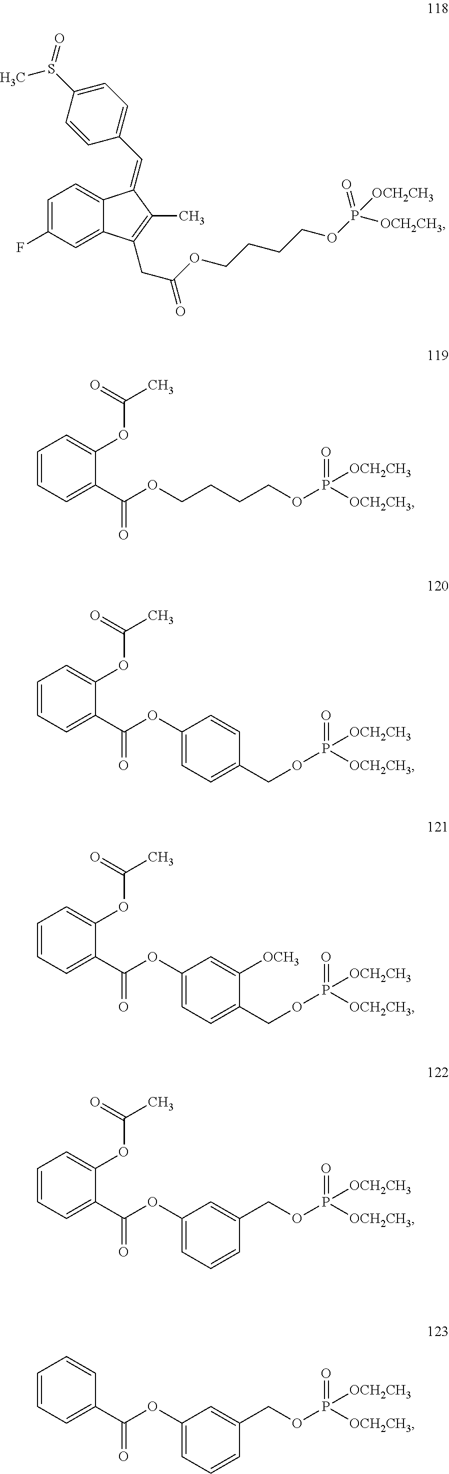 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)c1ccccc1OC(C)=O ; CCOP(=O)(OCC)OCc1ccc(OC(=O)c2ccccc2OC(C)=O)cc1 ; CCOP(=O)(OCC)OCc1ccc(OC(=O)c2ccccc2OC(C)=O)cc1OC ; CCOP(=O)(OCC)OCc1cccc(OC(=O)c2ccccc2OC(C)=O)c1 ; CCOP(=O)(OCC)OCc1cccc(OC(=O)c2ccccc2)c1]

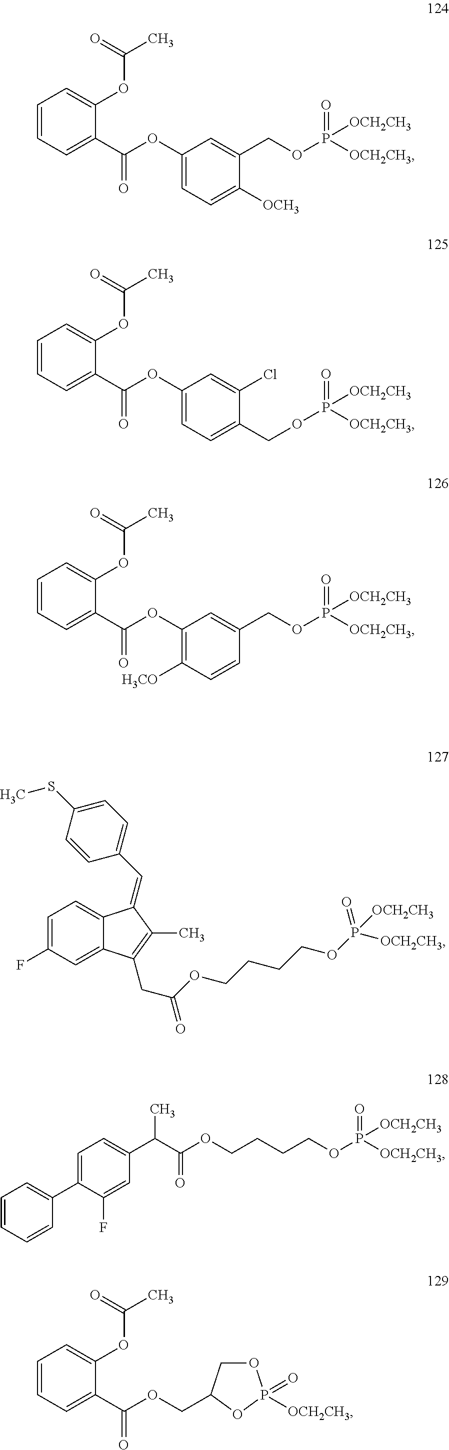 [SMILES: CCOP(=O)(OCC)OCc1cc(OC(=O)c2ccccc2OC(C)=O)ccc1OC ; CCOP(=O)(OCC)OCc1ccc(OC(=O)c2ccccc2OC(C)=O)cc1Cl ; CCOP(=O)(OCC)OCc1ccc(OC)c(OC(=O)c2ccccc2OC(C)=O)c1 ; CCOP(=O)(OCC)OCCCCOC(=O)CC1=C(C)/C(=C/c2ccc(SC)cc2)c2ccc(F)cc21 ; CCOP(=O)(OCC)OCCCCOC(=O)C(C)c1ccc(-c2ccccc2)c(F)c1 ; CCOP1(=O)OCC(COC(=O)c2ccccc2OC(C)=O)O1]

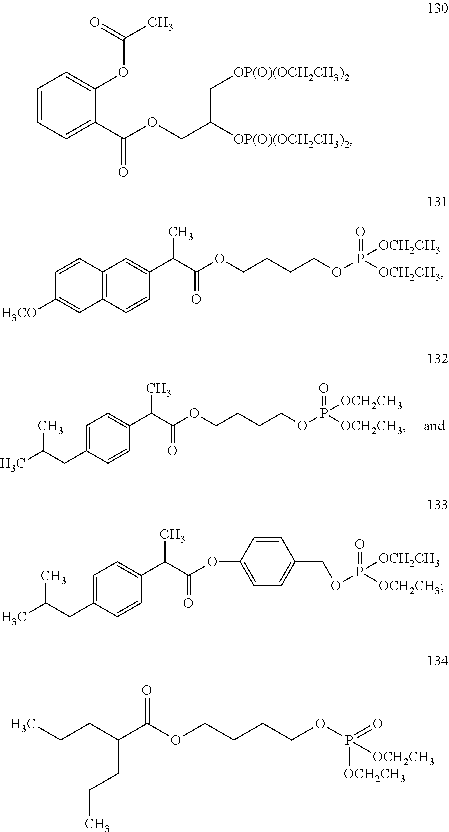

In a specific embodiment, the method further includes administering difluoromethylornithine and/or cimetidine to the subject, where the agents are administered within 28 days (e.g., within 21, 14, 10, 7, 5, 4, 3, 2, or 1 days) or within 24 hours (e.g., 12, 6, 3, 2, or 1 hours; or substantially concomitantly) of each other in amounts that together are effective to treat the subject.

In another embodiment, the compound is administered topically to the skin to treat non-cancerous conditions of the skin or mucous membranes.

In a further embodiment, the compound is administered in the form of a hydrogel or other nanocarrier.

In another embodiment, the hydrogel includes a poloaxamer and oleic acid.

In a specific embodiment, the compound is useful in the treatment of eczema or atopic dermatitis, dryness of the skin and recurring skin rashes, contact dermatitis, dyshidrosis, xerotic eczema, seborrhoeic dermatitis, neurodermatitis, discoid and venous eczema, actinic keratosis, papilloma (both cutaneous and anogenital), benign epithelial tumor, and hirsutism.

In yet another aspect, the present invention features methods for treating or preventing basal cell carcinoma, squamous-cell carcinoma, biliary tract cancer, bladder cancer, bone cancer, brain and other CNS cancer, cervical cancer, choriocarcinoma, connective tissue cancer, cancer of the digestive system, endometrial cancer, esophageal cancer, eye cancer, cancer of the head and neck, gastric cancer, intra-epithelial cancer, kidney cancer, larynx cancer, hairy cell leukemia, liver cancer, Hodgkin's and non-Hodgkin's lymphomas, melanoma, myeloma, neuroblastoma, oral cavity cancer (e.g. lip, tongue, mouth, pharynx), ovarian cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system said method comprising administering to a subject in need thereof a compound of Formula V.

In a further embodiment, an effective amount of a compound of Formula V is used to prevent a precancerous condition of the brain such as a precancerous brain lesion.

In a specific embodiment, the compound of Formula V is further described by Formula I, II, III, or IV or any specific compound described herein.

In another embodiment the compound of Formula V is a compound disclosed in U.S. Pat. No. 8,236,820, incorporated by reference.

In a further embodiment, the present invention features methods for the treatment of glioma.

A further aspect of the invention relates to a method of treating and/or preventing lung cancer and precancerous conditions of the lung, wherein said method comprises administering to a human or animal in need thereof, a pharmaceutically effective amount of a compound of the invention or the pharmaceutical composition thereof, wherein said administration is by the respiratory route.

In a specific embodiment, the method further includes administering one or more additional compounds having anticancer activity.

In another embodiment, the additional compound having anticancer activity is difluoromethylornithine, erlotinib, imatinib, or thiostrepton, where the agents are administered within 28 days (e.g., within 21, 14, 10, 7, 5, 4, 3, 2, or 1 days) or within 24 hours (e.g., 12, 6, 3, 2, or 1 hours; or concomitantly) of each other in amounts that together are effective to treat the subject.

In another aspect, the present invention relates to an effective amount of a compound of Formula V for use in the treating or reducing neuropathic pain, nociceptive pain, functional pain, musculo-skeletal pain, and central nervous system pain.

In a specific embodiment, the compound of Formula V is further described by Formula I, II, III, or IV or any specific compound described herein.

In another embodiment the compound of Formula V is a compound disclosed in U.S. Pat. No. 8,236,820, incorporated by reference.

In a further embodiment, the present invention features methods for treating subjects that have a predisposition or have been diagnosed with pain.

In yet another embodiment, the present invention relates to a compound of the invention for use as an antipyretic agent.

The pharmaceutical composition of the present invention may, for instance, be administered to a human or animal by nasal administration.

In some embodiments, the present invention relates to the pharmaceutical composition of the present invention, wherein said composition is administered to a human or animal in the form of an aerosol.

In a further embodiment, the pharmaceutical composition of the present invention is administered to a human or animal in the form of a dry powder aerosol.

The pharmaceutical composition of the present invention can be formulated in the form of nanoparticles. The nanoparticles may be lipid or polymeric nanoparticles or combinations thereof. Said nanoparticles may also be in form of a liposome, submicron emulsion, microemulsion, nanoemulsion, lipid micelle, solid lipid nanoparticle, polymeric micelle, polymeric nanoparticle or combinations thereof.

Pharmaceutical compositions of the present invention can comprise one or more further pharmaceutical agents in addition to one or more compounds of the invention. The compound of the invention can be administered alone or in combination with other active agents.

The pharmaceutical compositions of the present invention may be formulated with another additional compound having anticancer activity, for instance, with difluoromethylornithine or with tyrosine kinase inhibitors such as erlotinib or with compounds enhancing oxidative stress such as thiostrepton.

In yet another aspect, the present invention features methods for treating pain and/or fever. The invention further pertains to a method for alleviating pain, comprising administering to a subject in need thereof a pharmaceutically effective amount of a compound of the present invention or of a pharmaceutical composition of the present invention. The invention further pertains to a method for treating fever, comprising administering to a subject in need thereof a pharmaceutically effective amount of a compound of the present invention or of a pharmaceutical composition of the present invention.

In yet another embodiment of the present invention, the pharmaceutical composition is administered to a human or animal, in combination with tobacco smoke.

In a further aspect, the present invention is directed to an inhalation device comprising the pharmaceutical composition of the present invention.

Yet another aspect of the present invention is directed to a smoking device, for instance, to a cigarette, comprising tobacco and the pharmaceutical composition of the present invention.

In an embodiment of such a smoking device of the present invention, the pharmaceutical composition is spatially separated from the tobacco.

Such administration is effected via the inhalation device, or via the smoking device described in the application.

Still a further aspect of the invention relates to a product comprising a nicotine-containing material and an anti-cancer agent, wherein the anti-cancer agent comprises the compound of the invention.

In some embodiments, the anti-cancer agent may be an oxidative stress enhancer.

The anti-cancer agent may also comprise a combination of at least two different compounds having anti-cancer activity, i.e. a combination of curcumin and of the compound of the invention.

In one embodiment, the nicotine-containing material is tobacco leaf.

The product of the present invention contains nicotine and the anti-cancer agent in the ratio of from 1000:1 to 1:10 (wt:wt).

In some embodiments, the product is a smoking device selected from the group consisting of cigarette, cigar and smoking pipe, the smoking device optionally including an additional unit which renders the anti-cancer agent suitable for inhalation.

In other embodiments, the product is a smoking cessation product.

In some embodiments of the invention, the product is a transdermal patch.

In some further embodiments of the invention, the product is an inhalation device.

In some further embodiments of the invention, the product is an electronic cigarette.

In some further embodiments of the invention, the product is an orally applied product, for instance a smokeless tobacco product.

A further aspect of the invention relates to an anti-cancer agent for use in the prevention and/or treatment of cancer and/or precancerous conditions, wherein said anti-cancer agent is administered simultaneously with nicotine. The cancer may be, for instance, a lung cancer, brain cancer, or a precancerous condition thereof.

In one embodiment, the anti-cancer agent is inhaled together with tobacco smoke.

The compounds of the invention may be used for the manufacture of pharmaceutical compositions for treatment of a disease listed above.

The term “aliphatic substituent,” as used herein, includes saturated or unsaturated, branched or unbranched aliphatic univalent or bivalent substituents. In the present application, aliphatic substituent is intended to include, but is not limited to, alkyl, cycloalkyl, alkylene, alkenylene, alkynylene and alkadienylene substituents. According to the present invention, the aliphatic substituent has 1 to 100, (eg. 1 to 42 carbon atoms, 1 to 22 carbon atoms, 1 to 15 carbon atoms, 1 to 10 carbon atoms, 1 to 6 carbon atoms, for instance 4 carbon atoms). Exemplary aliphatic substituents are e.g. methylene, ethylene, trimethylene and tetramethylene.

The term “alkyl” used is the present application relates a saturated branched or unbranched aliphatic univalent substituent. The alkyl substituent has 1 to 100 carbon atoms, (eg. 1 to 22 carbon atoms, 1 to 10 carbon atoms 1 to 6 carbon atoms, 1 to 3 carbon atoms). Accordingly, examples of the alkyl substituent include methyl, ethyl, n-propyl, isopropyl, n-butyl, isobutyl, sec-butyl, tert-butyl, n-pentyl and n-hexyl.

The term “alkoxy” represents a chemical substituent of formula —OR, where R is an optionally substituted C1-C6 alkyl group, unless otherwise specified. In some embodiments, the alkyl group can be substituted, e.g., the alkoxy group can have 1, 2, 3, 4, 5 or 6 substituent groups as defined herein.

The term “alkoxyalkyl” represents a heteroalkyl group, as defined herein, that is described as an alkyl group that is substituted with an alkoxy group. Exemplary unsubstituted alkoxyalkyl groups include between 2 to 12 carbons. In some embodiments, the alkyl and the alkoxy each can be further substituted with 1, 2, 3, or 4 substituent groups as defined herein for the respective group.

As used herein, the term “cycloalkyl” refers to a monocyclic, bicyclic, or tricyclic substituent, which may be saturated or partially saturated, i.e. possesses one or more double bonds. Monocyclic substituents are exemplified by a saturated cyclic hydrocarbon group containing from 3 to 8 carbon atoms. Examples of monocyclic cycloalkyl substituents include cyclopropyl, cyclobutyl, cyclopentyl, cyclopentenyl, cyclohexyl, cyclohexenyl, cycloheptyl and cyclooctyl. Bicyclic fused cycloalkyl substituents are exemplified by a cycloalkyl ring fused to another cycloalkyl ring. Examples of bicyclic cycloalkyl substituents include, but are not limited to decalin, 1,2,3,7,8,8a-hexahydro-naphthalene, and the like. Tricyclic cycloalkyl substituents are exemplified by a cycloalkyl bicyclic fused ring fused to an additional cycloalkyl substituent.

The term “alkylene” used is the present application relates a saturated branched or unbranched aliphatic bivalent substituent (e.g. the alkylene substituent has 1 to 6 carbon atoms, 1 to 3 carbon atoms). Accordingly, examples of the alkylene substituent include methylene, ethylene, trimethylene, propylene, tetramethylene, isopropylidene, pentamethylene and hexamethylene.

The term “alkenylene” as used is the present application is an unsaturated branched or unbranched aliphatic bivalent substituent having a double bond between two adjacent carbon atoms (e.g. the alkenylene substituent has 2 to 6 carbon atoms, 2 to 4 carbon atoms). Accordingly, examples of the alkenylene substituent include but are not limited to vinylene, 1-propenylene, 2-propenylene, methylvinylene, 1-butenylene, 2-butenylene, 3-butenylene, 2-methyl-1-propenylene, 2-methyl-2-propenylene, 2-pentenylene, 2-hexenylene.

The term “alkynylene” as used is the present application is an unsaturated branched or unbranched aliphatic bivalent substituent having a triple bond between two adjacent carbon atoms (e.g. the alkynylene substituent has 2 to 6 carbon atoms 2 to 4 carbon atoms). Examples of the alkynylene substituent include but are not limited to ethynylene, 1-propynylene, 1-butynylene, 2-butynylene, 1-pentynylene, 2-pentynylene, 3-pentynylene and 2-hexynylene.

The term “alkadienylene” as used is the present application is an unsaturated branched or unbranched aliphatic bivalent substituent having two double bonds between two adjacent carbon atoms (e.g. the alkadienylene substituent has 4 to 10 carbon atoms). Accordingly, examples of the alkadienylene substituent include but are not limited to 2,4-pentadienylene, 2,4-hexadienylene, 4-methyl-2,4-pentadienylene, 2,4-heptadienylene, 2,6-heptadienylene, 3-methyl-2,4-hexadienylene, 2,6-octadienylene, 3-methyl-2,6-heptadienylene, 2-methyl-2,4-heptadienylene, 2,8-nonadienylene, 3-methyl-2,6-octadienylene, 2,6-decadienylene, 2,9-decadienylene and 3,7-dimethyl-2,6-octadienylene substituents.

The term “heteroaliphatic substituent”, as used herein, refers to a monovalent or a bivalent substituent, in which one or more carbon atoms have been substituted with a heteroatom, for instance, with an oxygen, sulfur, nitrogen, phosphorus or silicon atom, wherein the nitrogen and sulfur atoms may optionally be oxidized, and the nitrogen heteroatom may optionally be quaternized. The heteroatom(s) O, N and S may be placed at any interior position of the heteroaliphatic substituent. Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —CH₂—CH═N—OCH₃, and —CH═CH—N(CH₃)—CH₃. A heteroaliphatic substituent may be linear or branched, and saturated or unsaturated.

In one embodiment, the heteroaliphatic substituent has 1 to 100, (e.g 1 to 42 carbon atoms). In yet another embodiment, the heteroaliphatic substituent is a polyethylene glycol residue.

The term “polyethylene glycol residue” (PEG) refers to a compound of formula —(OCH₂CH₂)ₘR in which R is a hydrogen, alkyl, or alkoxy substituent and m has a value typically from 21 to 135, but not restricted to this range. Commercial polyethylene glycols having number average molecular weights of 1,000, 1,500, 1,540, 4,000 and 6,000 are useful in this invention. These solid polyethylene glycols have melting points of 35° C. to 62° C. and boiling or flash points ranging from 430° C. to over 475° C. Polyethylene glycol residues falling within the definition of the present invention include those having the formula —(OCH₂CH₂)ₘOCH₃ in which m is from 21 through 135, (e.g. 40 to 50).

As used herein, “aromatic substituent” is intended to mean any stable monocyclic, bicyclic or polycyclic carbon ring of up to 10 atoms in each ring, wherein at least one ring is aromatic, and may be unsubstituted or substituted. Examples of such aromatic substituents include phenyl, p-toluenyl(4-methylphenyl), naphthyl, tetrahydronaphthyl, indanyl, biphenyl, phenanthryl, anthryl or acenaphthyl. In cases where the aromatic substituent is bicyclic and one ring is non-aromatic, it is understood that attachment is via the aromatic ring.

The term “alkylaryl substituents” refers to alkyl substituents as described above wherein one or more bonds to hydrogen contained therein are replaced by a bond to an aryl substituent as described above. It is understood that an arylalkyl substituents is connected to the carbonyl group if the compound of the invention through a bond from the alkyl substituent. Examples of arylalkyl substituents include, but are not limited to, benzyl(phenylmethyl), p-trifluoromethylbenzyl(4-trifluoromethylphenylmethyl), 1-phenylethyl, 2-phenylethyl, 3-phenylpropyl, 2-phenylpropyl and the like.

The term “heteroaromatic substituent” as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic heteroaromatic substituents include phenyl, pyridine, pyrimidine or pyridizine rings that are
a) fused to a 6-membered aromatic (unsaturated) heterocyclic ring having one nitrogen atom;
b) fused to a 5- or 6-membered aromatic (unsaturated) heterocyclic ring having two nitrogen atoms;
c) fused to a 5-membered aromatic (unsaturated) heterocyclic ring having one nitrogen atom together with either one oxygen or one sulfur atom; or
d) fused to a 5-membered aromatic (unsaturated) heterocyclic ring having one heteroatom selected from O, N or S.

Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The aliphatic, heteroaliphatic, aromatic and heteroaromatic substituents can be optionally substituted one or more times, the same way or differently with any one or more of the following substituents including, but not limited to: aliphatic, heteroaliphatic, aromatic and heteroaromatic substituents, aryl, heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; CI; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(R_{X})₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, (alkyl)aryl or (alkyl)heteroaryl substituents described above and herein may be substituted or unsubstituted. Additionally, it will be appreciated, that any two adjacent substituents taken together may represent a 4, 5, 6, or 7-membered substituted or unsubstituted alicyclic or heterocyclic substituents. Additional examples of generally applicable substituents are illustrated by the specific embodiments shown below.

The terms “halo” and “halogen” refer to a halogen atom selected from the group consisting of F, Cl, Br and I.

The term “halogenated alkyl substituent” refers to an alkyl substituents as defined above which is substituted with at least one halogen atom. In an embodiment, the halogenated alkyl substituent is perhalogenated. In another embodiment, the halogenated alkyl substituent is a univalent perfluorated substituent of formula CₙF₂ₙ₊₁. For example, the halogenated alkyl substituent may have 1 to 6 carbon atoms, (e.g. 1 to 3 carbon atoms). Accordingly, examples of the alkyl group include trifluoromethyl, 2,2,2-trifluoroethyl, n-perfluoropropyl, n-perfluorobutyl and n-perfluoropentyl.

Some of the compounds of the present invention can comprise one or more stereogenic centers, and thus can exist in various isomeric forms, e.g. stereoisomers and/or diastereomers. Thus, the compounds of the invention and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided. Moreover, when compounds of the invention exist in tautomeric forms, each tautomer is embraced herein.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Non-cancerous skin and mucous membrane conditions that can be treated in accordance with this invention include, but are not limited to, the following conditions: warts, in particular genital warts, including perianal warts, penile warts and the like; pigmented benign skin tumors, e.g. seborrhoeic warts, dermatosis papulosa nigra, skin tags, lentigines (freckles), melanocytic naevi (congenital or acquired), and dermatofibroma; benign vascular tumors, e.g. cavernous haemangiomas (strawberry naevi), spider naevi, Campbell de Morgan spots (cherry haemangiomas), and pyrogenic granulomas; benign tumor papules, e.g. syringomas, apocrine hidrocystoma, milia, and sebaceous gland hyperplasia; benign tumor nodules, e.g. lipomas, epidermoid cysts, pilar cysts, pilomatrixoma, and poromas; benign tumor plaques, e.g. naevus sebaceous, epidermal naevi, and inflammatory linear verrucous epidermal naevus (ILVEN), psoriasis, actinic keratosis, any forms of hair loss, alopecia, eczema or atopic dermatitis, dryness of the skin and recurring skin rashes, contact dermatitis, dyshidrosis, xerotic eczema, seborrhoeic dermatitis, neurodermatitis, discoid and venous eczema, papilloma, benign epithelial tumor, and hirsutism.

The term “brain cancer” refers to both primary brain tumors and metastatic brain tumors that originate from non-brain cancer cells such as lung cancer cells. Primary brain tumors are categorized by the type of tissue in which they first develop. The most common brain tumors are called glioma; they originate in the glial tissue. There are a number of different types of gliomas: for instance, astrocytomas, brain stem gliomas, ependymomas, and oligodendrogliomas.

Primary brain tumors are categorized by the type of tissue in which they first develop. The most common brain tumors are called glioma; they originate in the glial tissue. There are a number of different types of gliomas: for instance, astrocytomas, brain stem gliomas, ependymomas, and oligodendrogliomas.

Other types of primary brain tumors which do not originate from the glial tissue are, for instance, meningiomas, craniopharyngiomas and germinomas.

In yet another embodiment, the pharmaceutical composition containing the compound of the invention can be useful in the treatment and/or prevention of cancer. “Cancer” as used herein refers to an uncontrolled growth of cells which interferes with the normal functioning of the bodily organs and systems. Cancers include, but are not limited to, basal cell carcinoma, biliary tract cancer; bladder cancer; bone cancer; brain and other central nervous system (CNS) cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemias, including hairy cell leukemia; liver cancer; lung cancer (e g small cell and non-small cell); lymphomas including Hodgkin's and nonHodgkin's lymphomas; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Cell proliferative disorders of the lung include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, precancerous lung lesion and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, emphysema, and Hodgkin's disease.

The expression “effective amount” as used herein, refers to a sufficient amount of the compound of the invention to exhibit the desired therapeutic effect. The exact amount required will vary from subject to subject, depending on the species, age, and general condition of the subject, the particular therapeutic agent and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the anticancer activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

As used herein, the term “lung cancer” includes all forms of cancer of the lung including, but not limited to malignant lung neoplasms, carcinoma in situ, typical carcinoid tumors, and atypical carcinoid tumors. Lung cancer can include small cell lung cancer (“SCLC”), non-small cell lung cancer (“NSCLC”), non-squamous non-small cell lung cancer, squamous non-small cell lung cancer, squamous cell carcinoma, non-squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructual heterogeneity (e.g. mixed cell types).

A metastasis is a region of cancer cells, distinct from the primary tumor location resulting from the dissemination of cancer cells from the primary tumor to other parts of the body. In one embodiment the cancer is melanoma (primary or metastatic). In one embodiment the cancer is breast cancer. In one embodiment the cancer is lung cancer. In one embodiment the cancer is prostate cancer. In one embodiment the cancer is colon cancer.

The phrase, “pharmaceutically acceptable derivative,” as used herein, denotes any pharmaceutically acceptable salt, ester, or salt or cocrystal of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others prodrugs. A prodrug is a derivative of a compound, usually with significantly reduced pharmacological activity, which contains at least one additional moiety, which is susceptible to removal in vivo yielding the parent molecule as the pharmacologically active species. An example of a prodrug is an ester, which is cleaved in vivo to yield a compound of interest. Prodrugs of a variety of compounds, and materials and methods for derivatizing the parent compounds to create the prodrugs, are known and may be adapted to the present invention. Certain exemplary pharmaceutical compositions and pharmaceutically acceptable derivatives will be discussed in more detail herein below.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are transformed in vivo to yield the parent compound of the above formula, for example by hydrolysis in blood. A thorough discussion is provided in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Some embodiments of the present invention are directed to the compound of the invention and pharmaceutical compositions thereof for prevention and/or treatment of precancerous conditions of the lung. The term “precancerous conditions in the lung” as used therein refers to a group of cell proliferative disorders of the lung.

According to the present invention, the compounds of the invention can be active against lung and/or brain cancer and therefore can be used in the treatment and/or prevention of lung and/or brain cancer and precancerous conditions thereof, wherein said compound is administered to a human or animal by the respiratory route. As used herein, “preventing,” “prevention,” or “prevent” describes reducing or eliminating the onset of lung or brain cancer or the precancerous conditions thereof or the symptoms or complications of lung and/or brain cancer and precancerous conditions thereof.

Treating lung and/or brain cancer can result in a reduction in size or volume of a tumor. A reduction in size or volume of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor or by any reproducible means of measurement.

Treating lung and/or brain cancer may further result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating lung and/or brain cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. A metastasis is a region of cancer cells, distinct from the primary tumor location resulting from the dissemination of cancer cells from the primary tumor to other parts of the body. The number of metastatic lesions may be measured by any reproducible means of measurement. The number of metastatic lesions may be measured by counting metastatic lesions visible to the naked eye or at a specified magnification. Preferably, the specified magnification is 2×, 10×, or 50×.

Treating lung and/or brain cancer can result in an increase in average survival time of a population of subjects treated according to the present invention in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with the compound of the invention.

Treating lung and/or brain cancer can also result in a decrease in the mortality rate of a population of treated subjects in comparison to an untreated population. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means, for example, by calculating for a population the average number of disease-related deaths per unit time following initiation of treatment with the compound of the invention. A decrease in the mortality rate of a population may also be measured, for example, by calculating for a population the average number of disease-related deaths per unit time following completion of a first round of treatment with the compound of the invention.

Another embodiment of the present invention relates to a method for preventing cancer by means of administering the compound of the invention or a pharmaceutical composition thereof. Accordingly, treatment of an individual with the compound of the invention or a pharmaceutical composition thereof reduces the risk of the individual to develop cancer. Preferably, after the treatment, the risk of the individual to develop cancer is reduced by 5% or greater; more preferably, the risk develop cancer is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. As used herein, reducing risk of developing cancer includes decreasing the probability or incidence of developing cancer for an individual compared to a relevant, e.g. untreated, control population, or in the same individual prior to treatment according to the invention. Reduced risk of developing cancer may include delaying or preventing the onset of a cancer. Risk of developing cancer can also be reduced if the severity of a cancer or a precancerous condition is reduced to such a level such that it is not of clinical relevance. That is, the cancer or a precancerous condition may be present but at a level that does not endanger the life, activities, and/or well-being of the individual. For example, a small tumor may regress and disappear, or remain static. Preferably, tumor formation does not occur. In some circumstances the occurrence of the cancer or the precancerous condition is reduced to the extent that the individual does not present any signs of the cancer or the precancerous condition during and/or after the treatment period.

The method for preventing cancer according to the present invention is beneficial both for individuals having a precancerous condition and individuals who are healthy. Individuals with lifestyle habits that could lead to cancer, particularly smokers, and individuals affected by diseases for which the probability of cancer incidence is high have a particularly high order of priority as individuals for the preventive method of the present invention. Furthermore, individuals who are likely to acquire familial cancers, and such individuals as those who are diagnosed with a risk of cancer by means of gene diagnoses based on single-nucleotide polymorphism or the like may also be targeted.

The compounds of the invention and pharmaceutical compositions thereof may have anticancer activity. Thus, the compounds represented by the invention and pharmaceutical compositions thereof may inhibit the growth of human or animal cancer cell lines such as A549 human lung cancer cells in in vitro tests and have IC₅₀ value of preferably less than 600 μM, more preferred of less than 100 μM, particularly preferred of less than 70 μM. The tests are preferably carried out as specified in S. Joseph et al. (Molecular Medicine Reports 2011, 4:891-899).

The compounds of the invention and pharmaceutical compositions thereof are further directed at individuals at risk of developing lung cancer. Such risk may be based on the medical or social history of an individual, such as inhalation of tobacco products as it occurs for example in smokers or exposure to asbestos or in non-smokers who breathe in secondhand smoke. Another category of individuals at risk for lung cancer are those harboring genetic mutations predisposing them to lung cancer. Yet another category is individuals who have been exposed to ionizing radiation or chemotherapeutic agents. Yet another category is individuals with a known cancer at a location other than the lungs that have a propensity to metastasize to the lungs.

Finally, another category is individuals with prior lung cancer that has already been treated. Accordingly, the corresponding embodiment of the present invention relates to a method for preventing cancer recurrence by means of administering the compound of the invention or a pharmaceutical composition thereof. Cancer recurrence is a re-development of the cancer in an individual, who had previously undergone a cancer treatment, after a period of time in which no cancer could be detected. The probability of a cancer recurring depend on many factors, including the type of cancer and its extent within the body at the time of the treatment.

The compounds of the present invention can have high in vivo stability. Preferably, the concentration the compound of the invention in blood plasma of an animal after 3 hr of administration is at least 30% of its initial concentration, more preferred at least 40% of its initial concentration, and particularly preferred at least 50% of its initial concentration. The corresponding tests can be carried out with animals such as mice according to the method described by Xie et al. (Xie G, Nie T, Mackenzie G, Sun Y, Huang L, Ouyang N, et al. Br. J. Pharmacol. 2011).

In addition, the compounds of the present invention can have cellular uptake values, which can be determined by using cancer cells, for instance human non-small cell lung cancer cells A549 and subsequently assaying their intracellular levels by HPLC. The tests can be performed according to the method outlined in Example 2. Preferably, the cellular uptake values of the compounds are higher than 0.1 nmol/mg protein, more preferred higher than 1.0 nmol/mg protein, even more preferred higher than 10.0 nmol/mg protein and particularly preferred higher than 50.0 nmol/mg protein.

In one embodiment, the compounds of the invention may have n-octanol-water partition coefficient (log P) value higher than 2, more preferred higher than 3 and particularly preferred higher than 4. Log P is defined as ratio of concentrations (mol/volume) of the compounds of the invention in n-octanol and in water. Suitable methods for the measurement of n-octanol-water coefficients are, for instance described in Octanol-Water Partition Coefficients: Fundamentals and Physical Chemistry, John Wiley and Sons Ltd., 1997, ISBN: 0-417-97397 1. Both solvents are mutually saturated before the measurement. At equilibrium the n-octanol phase contains 2.3 mol/l of water and the aqueous phase contains 4.5×10⁻³ mol/l of n-octanol. The measurement is carried out at the isoelectric point of the compound of the invention at temperature of 25° C. The log P of the compounds of the invention is preferably determined by the shake-flask method, which is, for example, described in the review of J. Sangster (J. Phys. Chem. Ref. Data 18, 1989; 3:1111-1227). The measurement is carried out under the conditions described by T. Fujita et al. (J. Am. Chem. Soc. 1964; 86:5175-5180) and the concentration of the compound of the invention in each of the two phases is determined by high performance liquid chromatography (HPLC).

In a further aspect, the invention is directed to a pharmaceutical composition comprising a compound of the invention, as described generally herein, and a pharmaceutically acceptable excipient. In a specific embodiment, the composition is useful in the treatment of human and animal inflammation related diseases including but not limited to rheumatologic diseases such as rheumatoid arthritis, osteoarthritis and Sjogren's syndrome; cardiovascular diseases, such as coronary artery disease, peripheral vascular disease and hypertension; neurodegenerative diseases such as Alzheimer's disease and its variants or cerebrovascular diseases; and autoimmune diseases such as lupus erythematosus; and other conditions characterized by chronic inflammation of organs such as the lung, such as chronic bronchitis or the sinuses, such as chronic sinusitis and inflammatory conditions of the gut such as inflammatory bowel disease; cardiovascular diseases, for example, coronary artery disease, peripheral vascular disease and hypertension; neurodegenerative diseases, for example, Alzheimer's disease and its variants or cerebrovascular diseases; and autoimmune diseases such as lupus erythematosus; other conditions characterized by chronic inflammation of organs such as the lung, such as chronic bronchitis or the sinuses, such as chronic sinusitis.

In yet another embodiment, the pharmaceutical composition containing the compound of the invention can be useful in the treatment and/or prevention of cancer and precancerous conditions, including but not limited to, benign prostatic hypertrophy, colon adenomas, actinic keratosis and various premalignant conditions of the lung, breast and pancreas.

The compounds of the invention may be useful in the treatment of the above-mentioned cancers. They are particularly suited for treating neoplastic and pre-neoplastic diseases of human and animal including but not limited to, for example, benign prostatic hypertrophy, prostate cancer, colon adenomas and colon cancer, cancer of the lung, lymphomas and leukemias. In another embodiment the cancer is skin cancer.

In another embodiment, the invention is directed to a method for inhibiting inflammation, in particular chronic inflammation in a subject in need thereof by administering to the subject an amount of the compound or composition of the present invention effective to inhibit inflammation. The subject may be a human patient or animal, for instance a mammal.

Thus, the invention is directed to the use of the aforementioned compounds for treating inflammation-related diseases and/or cancer.

The compounds and pharmaceutical compositions of the present invention can be further useful for alleviating (or mitigating) or treating a pain, for example, a chronic pain (particularly, a neuropathic pain) effectively. The compounds and pharmaceutical compositions of the present invention can for instance be administered as an injectable therapy adapted for one or more applications selected from a group consisting of subcutaneous, caudal, epidural, intramuscular, intradural, intraspinous and peripheral nerve blockade. They can further be formulated by entrapping in liposomes, lipid and polymeric micelles, dendrimers, solid lipid nanoparticles or other nanoparticles. The compounds and pharmaceutical compositions of the present invention are preferably capable of providing analgesic effect for at least 2 hours, more preferred for at least 4 hours, yet even more preferred for at least 6 hours.

Another aspect of the present invention features compounds of the invention and pharmaceutical compositions thereof for preventing and/or treating hyperthermia, fever, or pyresis in mammalian subjects. In various embodiments, the compounds and compositions of the present invention are effective for preventing elevation of body temperature above a normal body temperature range, and/or for lowering body temperature that has elevated above normal body temperature range in mammalian subjects suffering from impairment of thermal homeostasis.

By administering the antipyretic compound of the invention in a suitable prophylactic or therapeutic treatment protocol, subjects presenting with, or at elevated risk for, neuroleptic malignant syndrome or malignant hyperthermia can be effectively treated. Treatment of these conditions using the compounds and pharmaceutical compositions provided herein will reduce or prevent elevated temperatures in these subjects, and will often additionally substantially prevent or alleviate one or more of the above-identified symptoms associated with the subject condition as well.

Antipyretic agents are provided for effective management, prophylaxis, and/or treatment of various forms of “hot flashes” that occur in mammalian subjects. Hot flashes are most commonly associated with menopause, however, they may also be drug induced (for example by anti-estrogen compounds such as tamoxifen, toremifen and raloxifen), or triggered by removal of estrogen-producing tissues (e.g., after abdominal hysterectomy and bilateral salpingo-oopherectomy. As used herein, the term “hot flash” refers to any sudden, typically brief, sensation of heat, which often appears to affect the entire body, and may further be accompanied by secondary symptoms, including sweating, palpitations, and/or red blotching of the skin. In the exemplary case of menopausal hot flashes (i.e., menopausal, post-menopausal, and perimenopausal hot flashes) the antipyretic agents of the invention provided by the present invention are effective to substantially prevent or alleviate one or more of the foregoing symptoms.

Antipyretic effectiveness of the compounds of the present invention in this context may be demonstrated, for example, by a reduction in the number of hot flashes experienced by test versus control subjects, wherein the number of hot flashes of treated menopausal subjects may be reduced, for example, to fewer than 5 per day, fewer than 3 per day, fewer than 2 per day, fewer than 1 per day, or eliminated altogether. Alternatively, effectiveness may be demonstrated by a number of other numerical evaluation and scale rating systems including, but not limited to, the Kupperman Menopausal Index, the Menopause Rating Scale, Montgomery-Asberg Depression Rating Scale, the Hamilton Anxiety Rating Scale and the Hamilton Depression Rating Scale. Using the Hamilton Depression Rating Scale, for example, a score of 10-13 indicates mild depression; 14-17 mild to moderate depression; >17 moderate to severe depression. In the Hamilton Anxiety Rating Scale, mild anxiety is 18-24, moderate anxiety is 25-29 and severe anxiety would be any number over 30. With the Kupperman Menopausal Index is an assessment system that involves grading major menopausal symptoms from 0 (not present) to 3 (severe) and using the total score to quantify severity symptoms. The symptoms include hot flashes, depression, headache, palpitations, joint pain, loss of concentration, sleep disturbance, profuse perspiration, nervousness and irritability.

Hyperthermia is also common in cancer patients, either through infection, tumor development (causing paraneoplastic fever), drugs (allergic or hypersensitivity reactions), blood product transfusion, and graft-versus-host disease (GVHD). Paraneoplastic fever, or fever caused by tumors, is particularly common in patients presenting with lymphoma and renal cell carcinoma. These and other subjects are effectively treated, prophylactically and/or therapeutically, by administering to the subject an antipyretic effective amount of an antipyretic agent of the invention sufficient to prevent or reduce temperature elevation, as noted above, or to prevent or alleviate one or more related hyperthermic response(s) and/or one or more symptom(s) secondary or attendant to hyperthermia in the subject.

According to the present invention the compounds of the invention may possess anti-inflammatory activity, analgesic activity and/or anticancer activity.

In some embodiments compounds of the invention and pharmaceutical compositions thereof can reduce the levels of inflammatory cytokines such as tumor necrosis factor-α (TNF-α) by at least 50% in in vivo tests with female LEW/CrlBR Lewis rats when given in a daily dosage of preferably no more than 500 mg/kg, more preferred of no more than 300 mg/kg, particularly preferred of no more than 100 mg/kg. The tests are preferably performed according to the procedure by L. Huang et al. (British Journal of Pharmacology 2011; 162:1521-1533).

The compounds of the invention can have anticancer activity. For example, the compounds of the invention can inhibit the growth of human or animal cancer cell lines such as HT-29 in in vitro tests and have IC₅₀ value of preferably less than 300 μM, more preferred of less than 100 μM, particularly preferred of less than 70 μM. The tests are preferably carried out as specified in S. Joseph et al. (Molecular Medicine Reports 2011, 4:891-899).

In a specific embodiment, the invention is directed to a method for obtaining a pharmaceutical composition, comprising formulating the compounds of the present invention into a composition comprising the compound of the present invention and one or more pharmaceutically acceptable carrier or excipient. The invention is further directed to uses of the compound of the present invention for manufacturing a medicament.

Other features and advantages of the invention will be apparent from the following detailed description, figures and the claims.

### BRIEF DESCRIPTION OF THE FIGURES

FIG. 1 is an illustration of a nose-only aerosol exposure system.

FIGS. 2A-2F are illustrations of modes of administration of the compound of the invention.

FIG. 3 is a graph that illustrates the biodistribution of liposomal phospho-ibuprofen amide in mice after i.v. administration at 200 mg/kg.

FIG. 4 is a graph that illustrates the inhibition of human lung cancer by phospho-ibuprofen amide.

FIG. 5 is a graph that displays the inhibition of human lung cancer by phospho-ibuprofen amide.

FIG. 6 is a graph that illustrates the pharmacokinetic study of PTI in mice.

FIG. 7 is a graph that illustrates effective inhibition of human cancer cell xenograft tumor growth by PTI.

FIG. 8 is a graph that illustrates levels of phospho-sulindac (PS) and its metabolites in the lungs (A) and plasma (B) of mice subjected to aerosol administration of PS.

FIG. 9 is a graph that illustrates survival rates of control and aerosolized-PS treated groups of mice implanted orthotopically with A549 cells.

FIG. 10 is an image that illustrates aerosol administration of PS.

FIG. 11 is a graph that illustrates aerosol administration of PS.

FIG. 12 is a graph that illustrates lung levels of PS after inhalation and oral administration.

FIG. 13 is a graph that illustrates the plasma level of PS after inhalation and oral administration.

FIG. 14 is a graph that illustrates that phosphovalproic acid (PV) and ibuprofen phospho-glycerol amide (PGIA) synergize strongly to inhibit the growth of glioblastoma and lung cancer

FIG. 15 is a graph that illustrates biodistribution of liposomal phospho-ibuprofen amide in mice. The concentration was determined in the major organs of mice (n=2), 1 h after iv administration of 200 mg/kg of phospho-ibuprofen amide 1.

FIG. 16 is a graph that illustrates inhibition of human lung cancer by phospho-ibuprofen amide. Mice xenografted with A549 human non-small cell lung cancer cells were treated with liposomal phospho-ibuprofen amide 1, ibuprofen or vehicle by I.V. as indicated. Representative fluorescence images of lungs from control (left), ibuprofen (center) and phospho-ibuprofen amide 1 (left) treated mice. These images indicate the efficacy of these compounds; phospho-ibuprofen amide 1 essentially eliminated lung cancer, with very few foci of cancer cells. Values (% control) are Mean±SEM.

FIG. 17 is a graph that illustrates inhibition of human lung cancer by phospho-ibuprofen amide. Lung weight, g. Results are from the study described in FIG. 2. Lung weight includes both noncancerous tissue and cancerous tissue and thus underestimates the effect of the test agent.

FIG. 18 are HPLC chromatograms of extracts from cells treated with ibuprofen, PI bearing phosphate and PI bearing diethylphosphate. The vertical lines indicate the respective position in the chromatograms of the peaks of authentic compounds. PI phosphate and ibuprofen generated no discernible peaks.

FIG. 19 is a graph that illustrates pharmacokinetic study of phosphosulindac amide (PSA) and sulindac. 100 mg/kg PSA or 62 mg/kg sulindac (equimolar to PSA) were administered to mice as a single oral gavage dose in corn oil and blood samples were collected at the indicated time points starting at 15 min post injection. Plasma levels of the PSA or sulindac metabolites (sulindac sulfide and sulindac sulfone) were determined. Values are the average of duplicate samples (all within 12% of each other).

FIG. 20 is a graph that illustrates colon cancer growth inhibition by PSA. Left: PSA inhibited human colon cancer cell xenograft tumor growth. Mice with SW480 human colon cancer xenografts were treated with PSA 100 mg/kg/day or vehicle (corn oil) by oral gavage. Right: Effect of PSA on tumor multiplicity in Apc^{Min/+} mice. The total number of tumors per animal was reduced after PSA treatment by 85%. Values are Mean±SEM.

FIG. 21 is a graph that illustrates toxicity assessment of phospho-tyrosyl-indomethacin (PTI). Left: Representative H&E stained gastric tissue sections from control, indomethacin (Indo) or PTI treated mice. Indo caused gastric damage but not PTI. The numerical results are shown below. Right: PTI shows no genotoxicity; TA98. Salmonella typhimurium strain.

FIG. 22 is a graph that illustrates pharmacokinetic study of PTI in mice. Following a single i.p. dose of 100 mg/kg PTI (left) or 58 mg/kg indomethacin (equimolar to PTI) (Indo; right) the plasma levels of intact PTI and indomethacin (hydrolysis product of PTI) were determined at the indicated time points. The AUCₜₒₜₐₗ of PTI is about 3.5 times higher than that of indomethacin.

FIG. 23 is a graph that illustrates effective inhibition of human cancer cell xenograft tumor growth by PTI. Mice with A549 human non-small cell lung cancer or SW480 human colon cancer xenografts were treated with PTI 10 or 15 mg/kg/day or vehicle (corn oil) by oral gavage as indicated. Mice with lung cancer xenografts followed a treatment protocol (treatment started when xenografts reached an average volume of 100 mm³) whereas those with SW480 xenografts followed a prevention protocol (drug administration started 1 wk prior to cell implantation). Values are Mean±SEM.

FIG. 24 is a graph that illustrates pharmacokinetic study of PEGylated phospho-ibuprofen (PI-PEG) and phospho-ibuprofen (PI) in mice. X-axis: time, hours; y-axis: PI-PEG concentration, μM.

FIG. 25 is a graph that illustrates tumor volumes of SW-480 xenografts in nude mice treated with PBS (control) or with PI-PEG (n=9 tumors/group).

FIG. 26 is an image that illustrates the growth inhibitory effect of PTI.

FIG. 27 is graph that illustrates the stability of PTI to esterases.

FIG. 28 is a graph that illustrates the cell uptake of PTI.

FIG. 29 is a graph that illustrates in vivo efficacy against a gastric cancer model.

FIG. 30 is a graph that illustrates in vivo efficacy against a skin cancer model.

FIG. 31 is a graph that illustrates in vivo efficacy against a lung cancer model.

FIG. 32 is a graph that illustrates in vivo efficacy against a breast cancer model.

FIG. 33 is a graph that illustrates that topical PS/DFMO inhibits skin papillomas. Left: Frequency of papillomas by histology Pap1-3: papilloma grade 1-3; M1-3: microinvasion grade 1-3; SCC: squamous cell carcinoma. Upper right: Representative pictures of control and treated mice. Lower right. Epidermis thickness of topical treatment groups. Blue arrows point to the epidermis; markedly thickened in vehicle, it is normalized in PS/DFMO. *, p<0.001 from vehicle.

FIG. 34 is a graph that illustrates the effect of oral and topical PS and DFMO on papillomas. Treatment of mice with chemically-induced papillomas started on wk 11, Upper: Tumor multiplicity during treatment. Lower: Tumor load/mouse at sacrifice. *, p<0.001 from vehicle.

FIG. 35 is an image that illustrates the components of the Franz cell. The hydrogel is placed on the skin in the donor chamber. The drug reaches the solvent-filled receptor chamber (stirring) and samples are obtained at various time points. The heating jacket keeps the temperature constant.

### DETAILED DESCRIPTION OF THE INVENTION

### Compounds

The invention features compounds and pharmaceutical compositions for the prevention and/or treatment of lung and brain cancer and precancerous conditions thereof, for the treatment of pain, for the treatment of skin disorders, for treating and/or preventing inflammation-related diseases, and for the treatment and prevention of cancer. Exemplary compounds described herein are compounds that have a structure according to the Formula I, II, III, IV, or V, shown below.

Formula I is provided below.

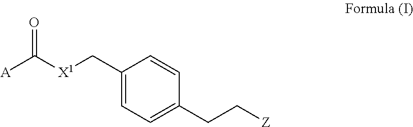

In Formula I: A is an optionally substituted aliphatic, heteroaliphatic, aromatic, heteroaromatic substituent or alkylaryl substituent having 1 to 100 carbon atoms or is selected from:

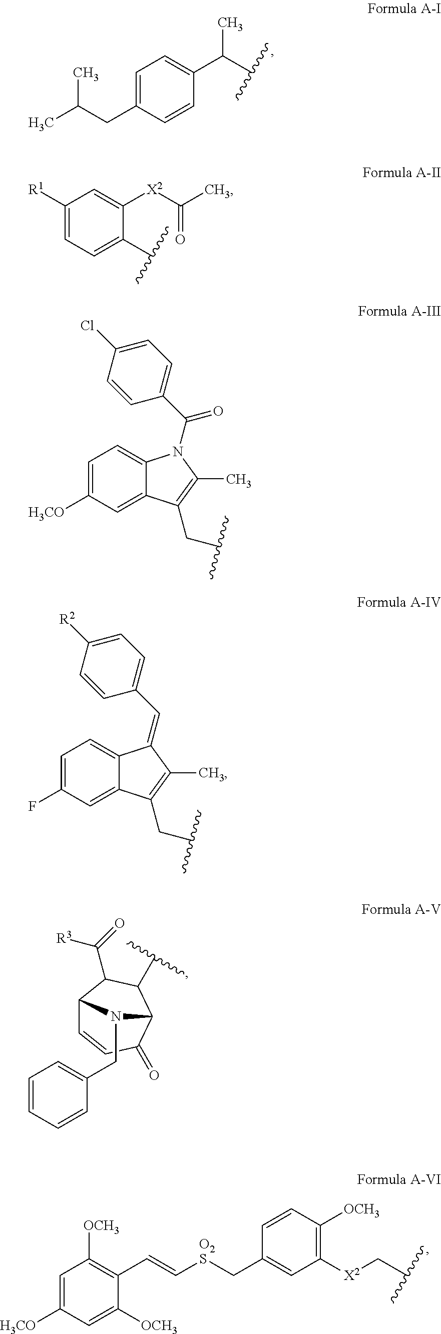

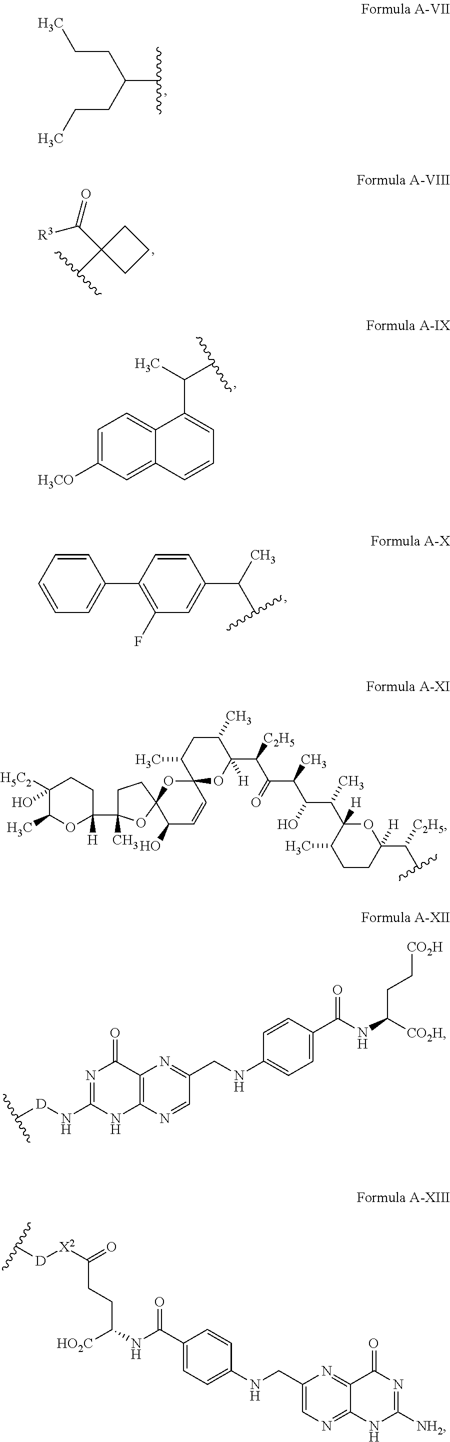

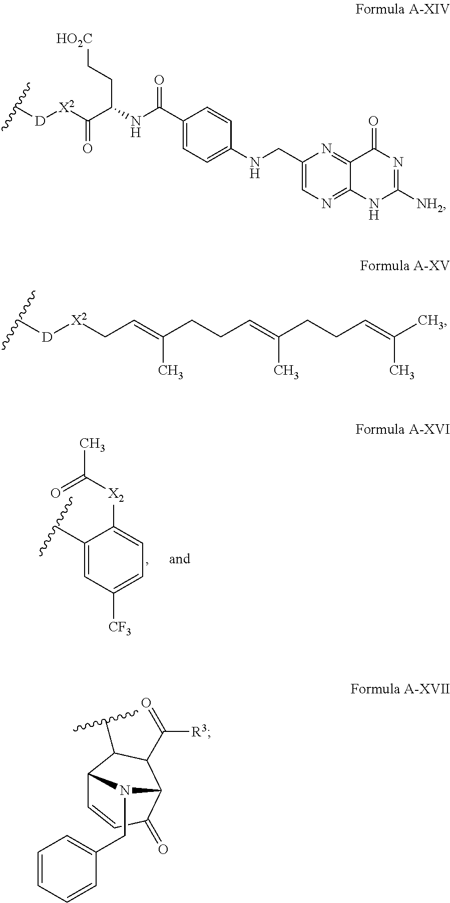

D is absent or

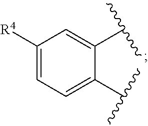

X¹ and X² are independently selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, —X¹—(CH₂)₄-Z, and

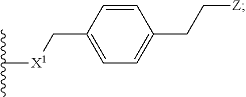

R⁵ is selected from hydrogen and C₁₋₆ alkyl;

Z is selected from:

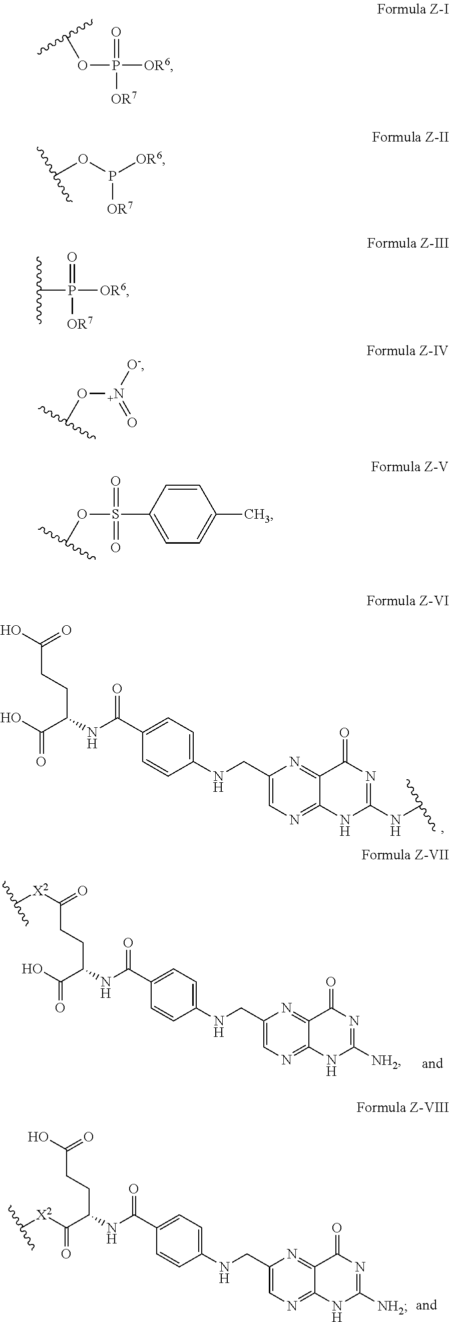

R⁶ and R⁷ are independently selected from hydrogen, C₁₋₆-alkyl, and polyethylene glycol residue.

In some embodiments, X¹ is —NR⁵—, and R⁵ is selected from hydrogen, methyl, and ethyl.

In other embodiments, X¹ is —O—.

In certain embodiments, Z is

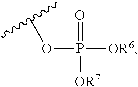

R⁶ is selected from ethyl and a polyethylene glycol residue, and R⁷ is selected from hydrogen and ethyl.

In still other embodiments, A is selected from:

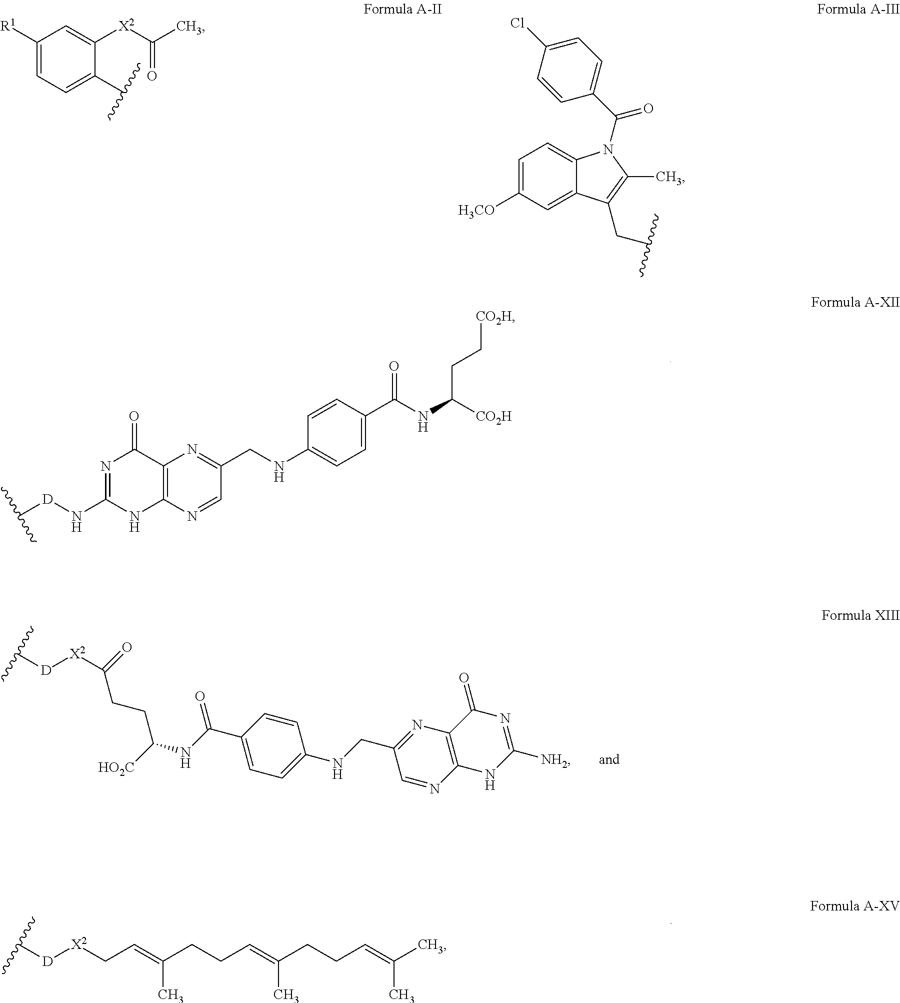

wherein D is

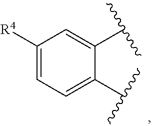

R¹ and R⁴ are independently selected from hydrogen and trifluoromethyl, and X² is selected from —O—, —S—, and —NH—.

In some embodiments, X¹ is —O—, Z is —O—P(O)(CH₂CH₃)₂, and A is:

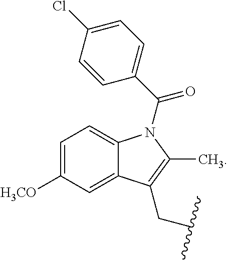 [SMILES: *Cc1c(C)n(C(=O)c2ccc(Cl)cc2)c2ccc(OC)cc12]

In certain embodiments, X¹ is selected from —O— and —NH—, Z is —O—P(O)(CH₂CH₃)₂, A is:

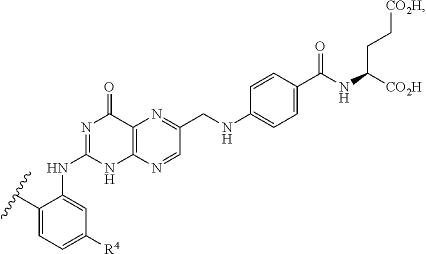

and R⁴ is selected from hydrogen and trifluoromethyl.

In other embodiments, X¹ and X² are independently selected from —O— and —NH—, Z is —O—P(O)(CH₂CH₃)₂, A is:

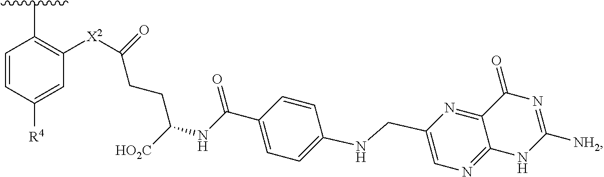

and R⁴ is selected from hydrogen and trifluoromethyl.

In some embodiments, X¹ and X² are independently selected from —O—, —S—, and —NH—; Z is —O—P(O)(CH₂CH₃)₂; and A is:

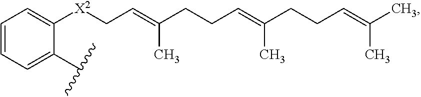

In some embodiments, X¹ is selected from —O—, —S—, and —NH—, Z is selected from —O—P(O)(CH₂CH₃)₂ and —ONO₂, A is:

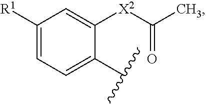

and R¹ is selected from hydrogen and trifluoromethyl, and X² is selected from —O—, —S— and —NH—.

In certain embodiments, X¹ is selected from —O— and —NH—, Z is —ONO₂, and A is:

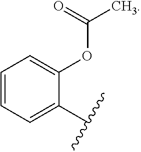 [SMILES: *c1ccccc1OC(C)=O]

Accordingly, the compounds of Formula I include but are not limited to compounds 1 to 21 and 109 specified above.

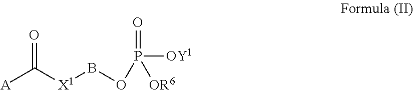

or a pharmaceutically acceptable salt thereof.

In Formula II: Y¹ is a polyethylene glycol residue;

R⁶ is selected from hydrogen, C₁₋₆-alkyl, and polyethylene glycol residue;

A is an optionally substituted aliphatic, heteroaliphatic, aromatic, heteroaromatic substituent or alkylaryl substituent having 1 to 100 carbon atoms or selected from:

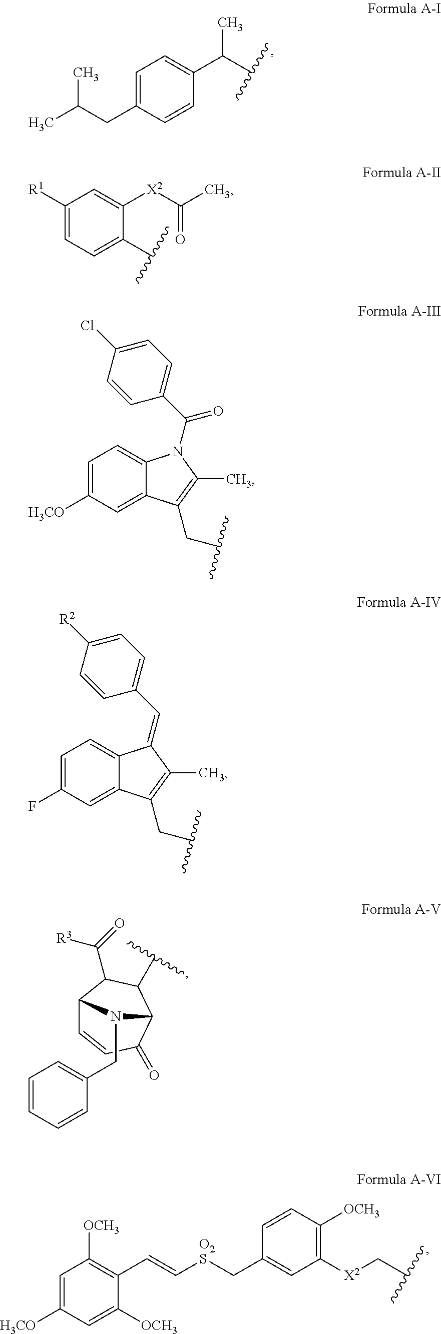

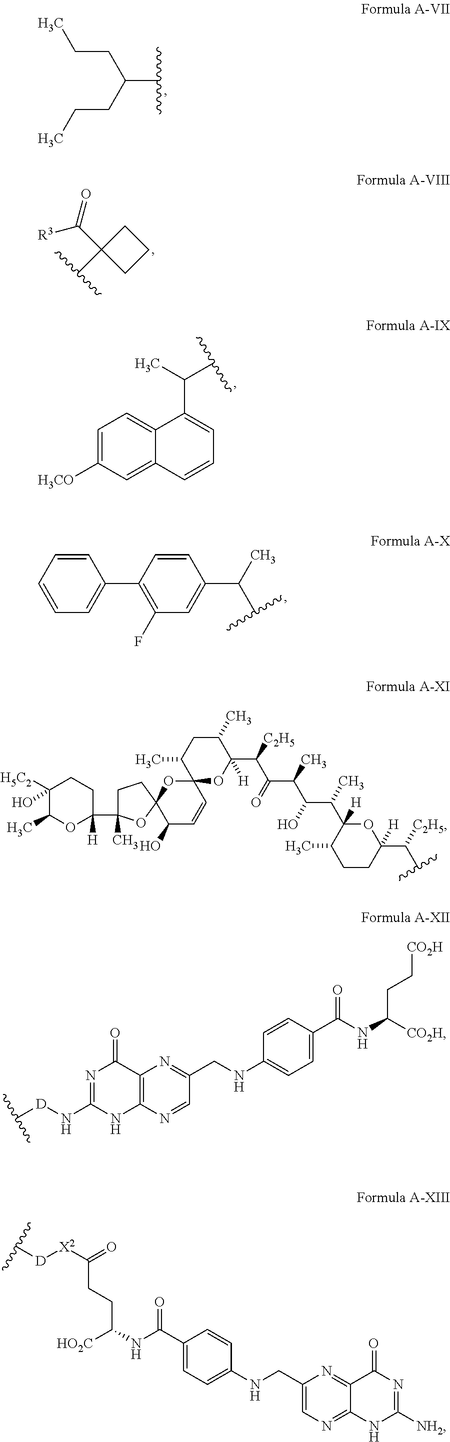

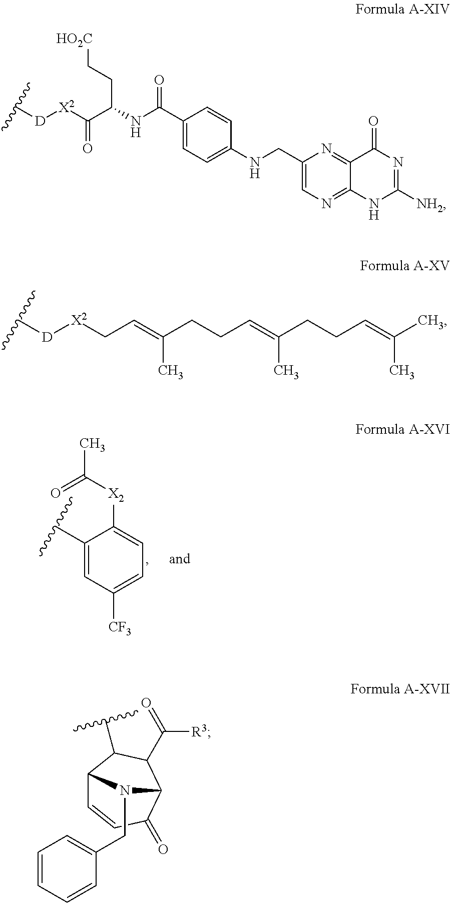

D is absent or

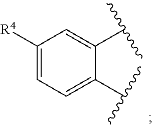

X¹ and X² are independently selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B-Z;

R⁵ is selected from hydrogen and C₁₋₆ alkyl;

B is selected from:

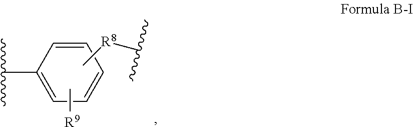

a single bond, and an aliphatic group with 1 to 22 carbon atoms;

R⁸ is a C₁₋₄ alkylene; and

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl.

In further embodiments, Y¹ is a polyethylene glycol residue described by —O(CH₂CH₂O)ₘR¹⁰, wherein m is 1 to 100 (e.g. 20 to 100, 20 to 50, 40 to 50), and R¹⁰ is selected from hydrogen, alkyl and alkoxy, and R⁶ is hydrogen.

In still other embodiments, Y¹ is —O(CH₂CH₂O)ₘR¹⁰ wherein m is 45, R¹⁰ is —OCH₃, and R⁶ is hydrogen.

In some embodiments, X¹ is —O—.

In other embodiments, X¹ is —NR⁵— and R⁵ is selected from hydrogen, methyl, and ethyl.

In certain embodiments, B is —(CH₂)₄—.

In some embodiments, A is:

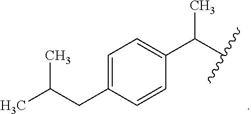 [SMILES: *C(C)c1ccc(CC(C)C)cc1]

In other embodiments, X¹ is —O—, B is —(CH₂)₄—, Y¹ is —O(CH₂CH₂O)ₘR¹⁰ wherein m is 45 and R¹⁰ is —OCH₃, R⁶ is hydrogen, and A is:

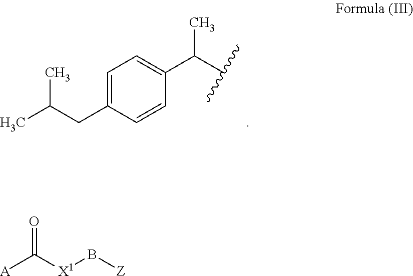

or a pharmaceutically acceptable salt thereof.

In Formula III: A is selected from:

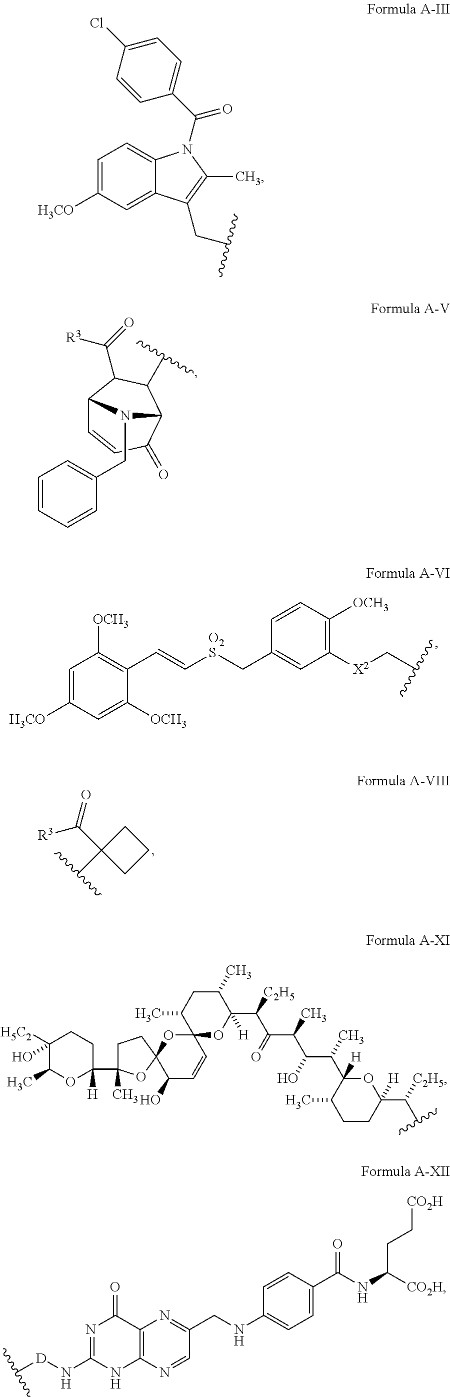

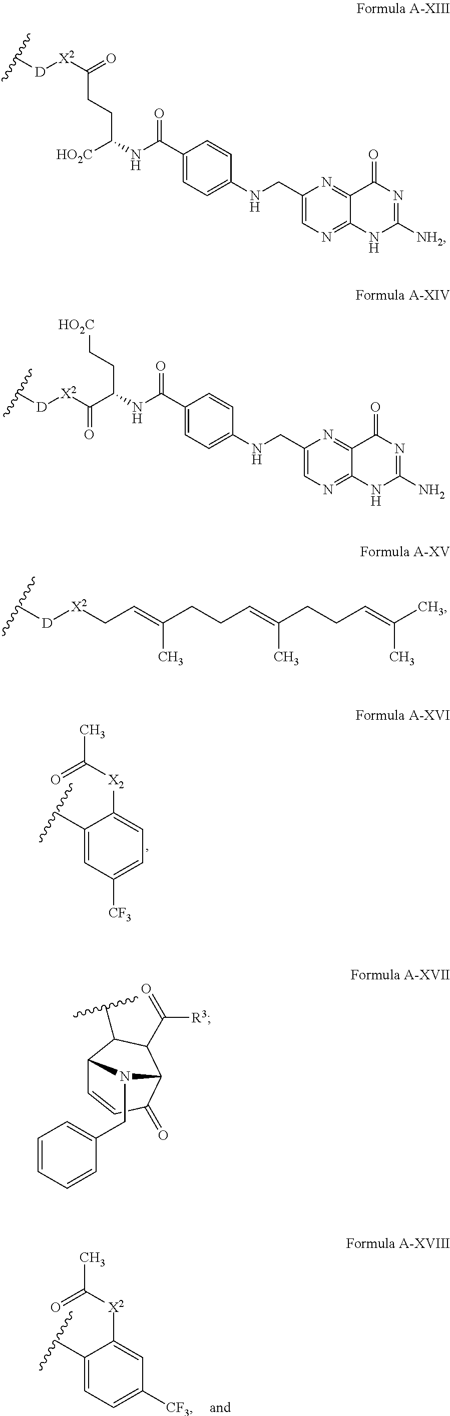

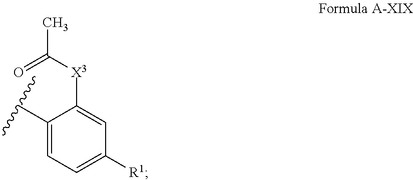

D is absent or

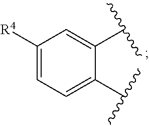

X¹ and X² are independently selected from —O—, —NR⁵—, and —S—;

R¹ and R⁴ are independently selected from hydrogen and trifluoromethyl;

X³ is selected from —S— and —NH—;

R³ is selected from hydroxyl, Z, and —X¹—B-Z;

R⁵ is selected from hydrogen and C₁₋₆ alkyl;

B is selected from:

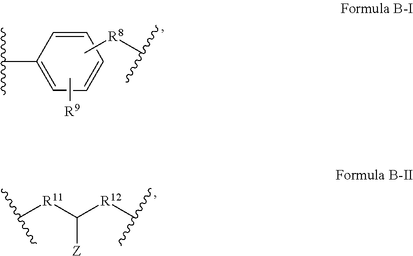

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;

Z is selected from:

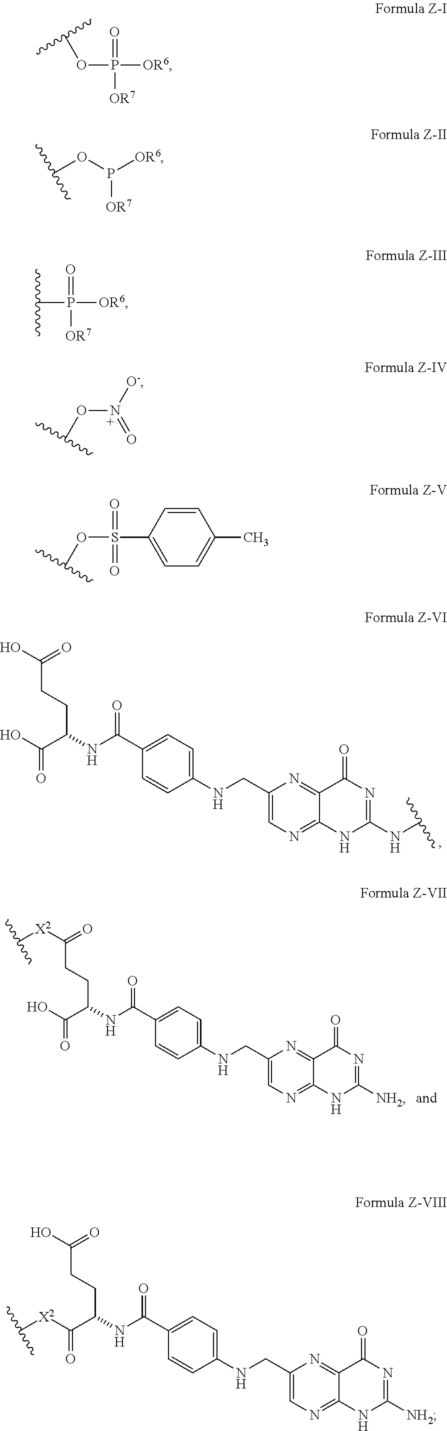

or B together with Z forms a structure:

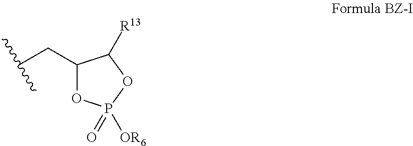

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

In still other embodiments, X¹ is —O—.

In certain embodiments, X¹ is —NR⁵— and R⁵ is selected from hydrogen, methyl, and ethyl.

In some embodiments, B is selected from:

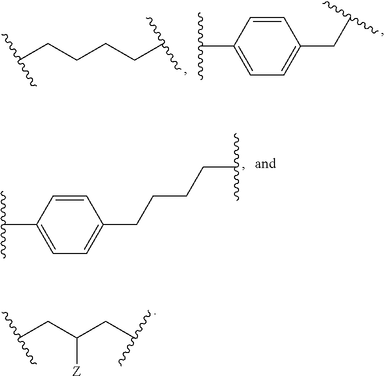

In other embodiments, Z is selected from —OP(O)(OCH₂CH₃)₂ and —ONO₂.

In further embodiments, BZ is

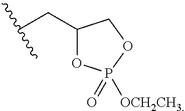 [SMILES: *CC1COP(=O)(OCC)O1]

In certain embodiments, X¹ is selected from —O— and —NH—, B is selected from

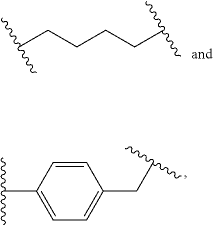 [SMILES: *CCCC* ; *Cc1ccc(*)cc1]

### Z is —OP(O)(OCH₂CH₃)₂, and A is:

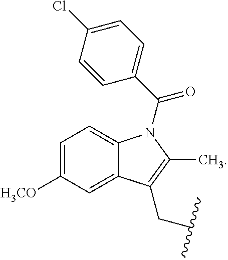 [SMILES: *Cc1c(C)n(C(=O)c2ccc(Cl)cc2)c2ccc(OC)cc12]

In some embodiments, X¹ is selected from —O— and —NH—, B is selected from

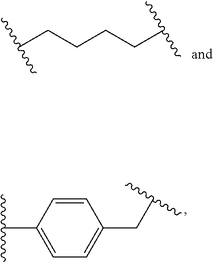 [SMILES: *CCCC* ; *Cc1ccc(*)cc1]

### Z is —OP(O)(OCH₂CH₃)₂, A is

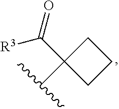

### and R³ is:

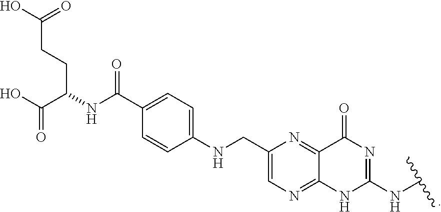

In some embodiments, wherein X¹ is selected from —O— and —NH—, B is selected from

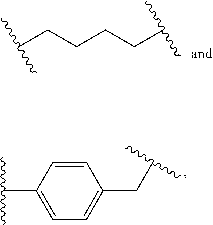 [SMILES: *CCCC* ; *Cc1ccc(*)cc1]

### Z is —OP(O)(OCH₂CH₃)₂, A is:

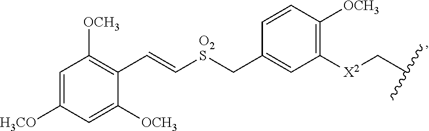

and X² is selected from —O— and —NH—.

In other embodiments, X¹ is selected from —O— and —NH—, B is selected from

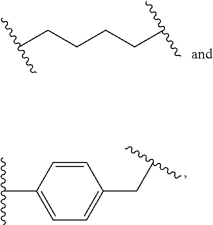 [SMILES: *CCCC* ; *Cc1ccc(*)cc1]

### Z is —OP(O)(OCH₂CH₃)₂, and A is:

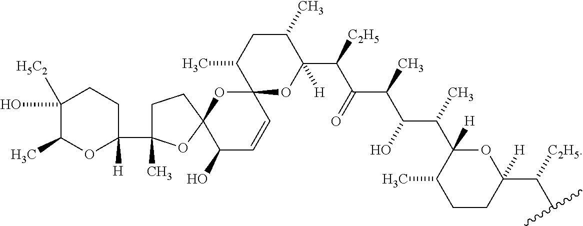

In further embodiments, X¹ is selected from —O— and —NH—, B is selected from

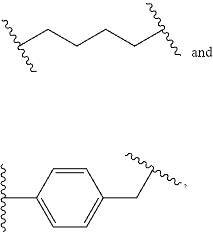 [SMILES: *CCCC* ; *Cc1ccc(*)cc1]

### Z is —OP(O)(OCH₂CH₃)₂, A is:

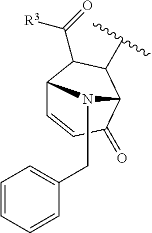

and R³ is hydroxyl or selected from:

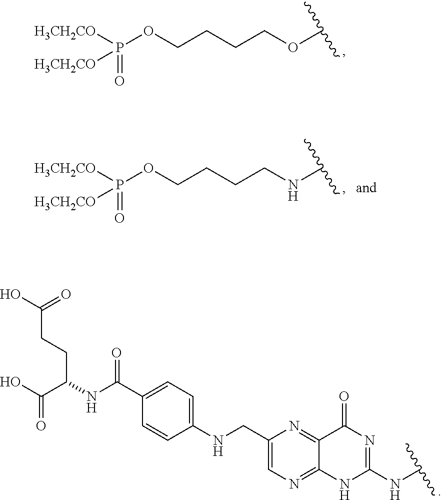

In certain embodiments, X¹ is selected from —O— and —NH—, B is selected from

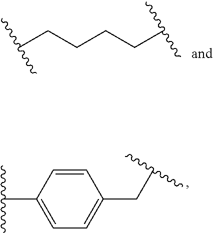 [SMILES: *CCCC* ; *Cc1ccc(*)cc1]

### Z is —OP(O)(OCH₂CH₃)₂, A is:

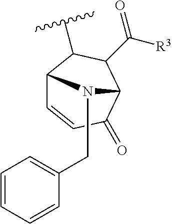

and R³ is hydroxyl or selected from:

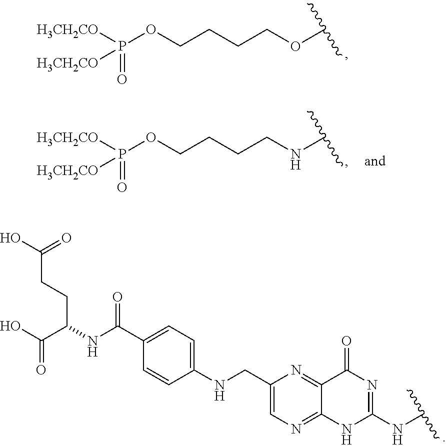

In some embodiments, X¹ is selected from —O— and —NH—, B is selected from

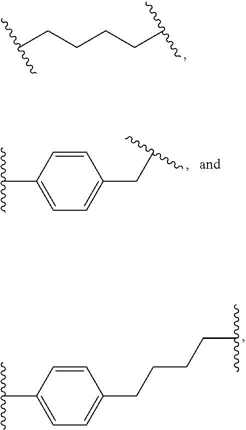 [SMILES: *CCCC* ; *Cc1ccc(*)cc1 ; *CCCCc1ccc(*)cc1]

### Z is —OP(O)(OCH₂CH₃)₂, A is:

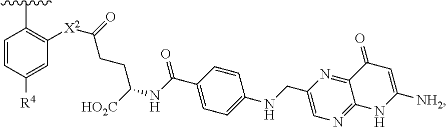

and R⁴ is selected from hydrogen and trifluoromethyl.

In some embodiments, X¹ is selected from —O— and —NH—, B is selected from

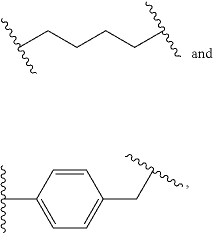 [SMILES: *CCCC* ; *Cc1ccc(*)cc1]

### Z is —OP(O)(OCH₂CH₃)₂, A is:

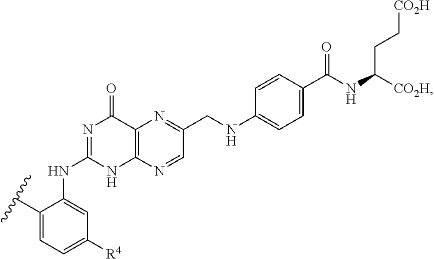

and R⁴ is selected from hydrogen and trifluoromethyl.

In other embodiments, X¹ is selected from —O— and —NH—, B is selected from

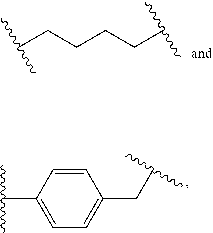 [SMILES: *CCCC* ; *Cc1ccc(*)cc1]

### Z is —OP(O)(OCH₂CH₃)₂, A is:

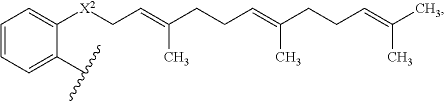

and X² is selected from —O—, —S—, and —NH—.

In other embodiments, X¹ is selected from —O— and —NH—, B is selected from

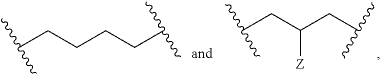

Z is selected from —OP(O)(OCH₂CH₃)₂ and —ONO₂, A is:

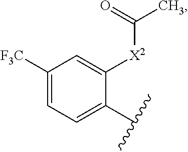

and X² is selected from —O—, —S—, and —NH—.

In some embodiments, X¹ is selected from —O— and —NH—, B is —(CH₂)₄—, Z is —ONO₂, A is:

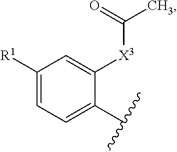

R¹ is selected from hydrogen and trifluoromethyl, and X³ is selected from —S—, and —NH—.

In other embodiments, X¹ is —NH—, A is:

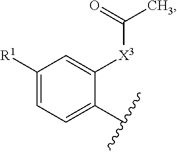

R¹ is selected from hydrogen and trifluoromethyl, and X³ is selected from —S—, and —NH—.

Accordingly, the compounds of Formula III include but are not limited to compounds 22 to 92, 108, and 112 to 116 specified above.

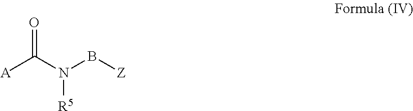

or a pharmaceutically acceptable salt thereof.

In Formula IV: A is an optionally substituted aliphatic, heteroaliphatic, aromatic, heteroaromatic substituent or alkylaryl substituent having 1 to 100 carbon atoms or selected from:

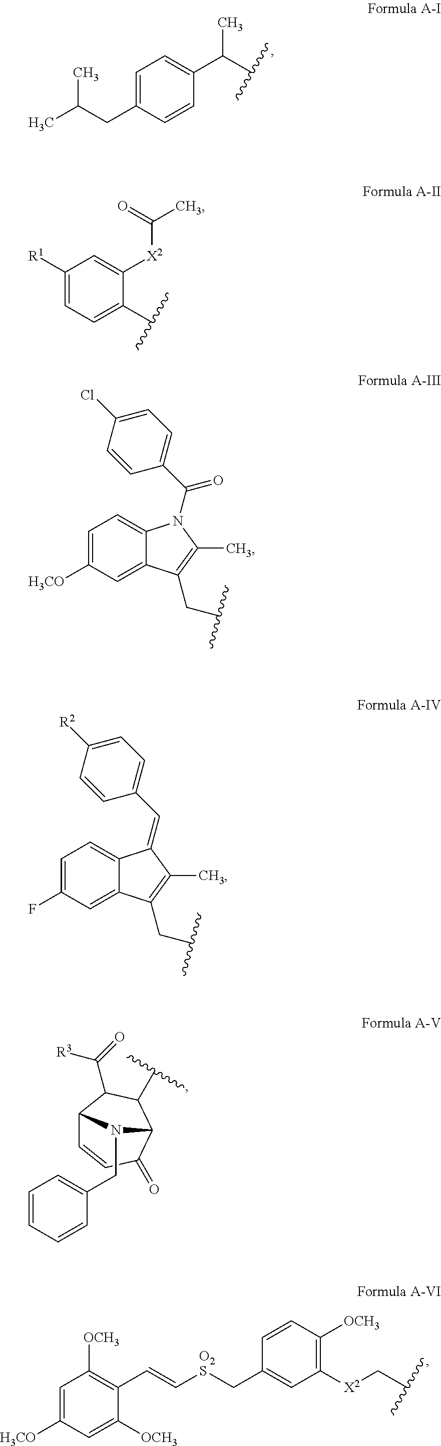

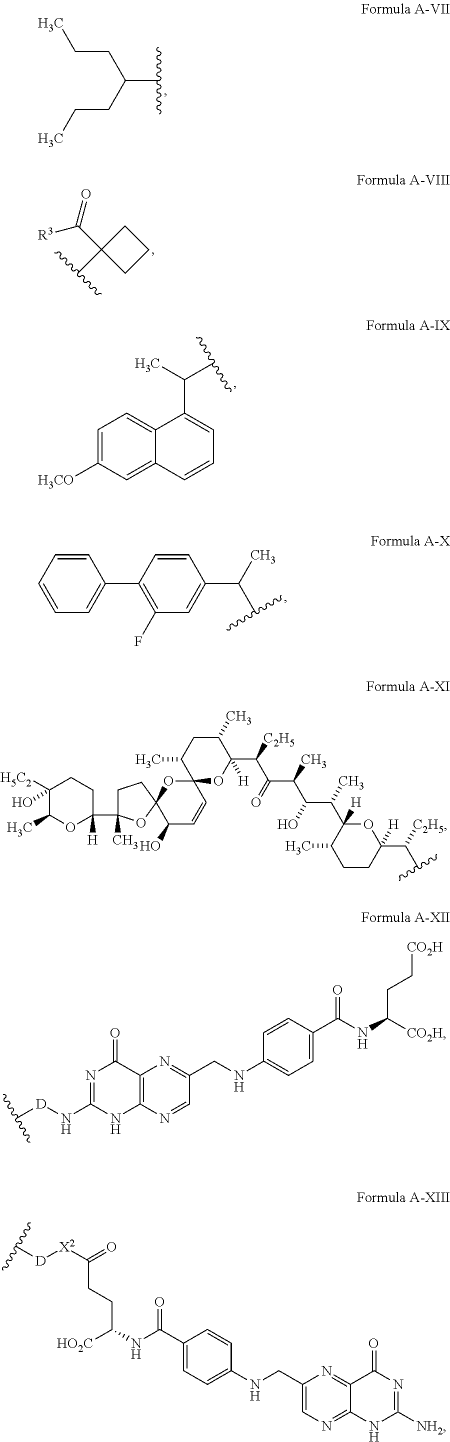

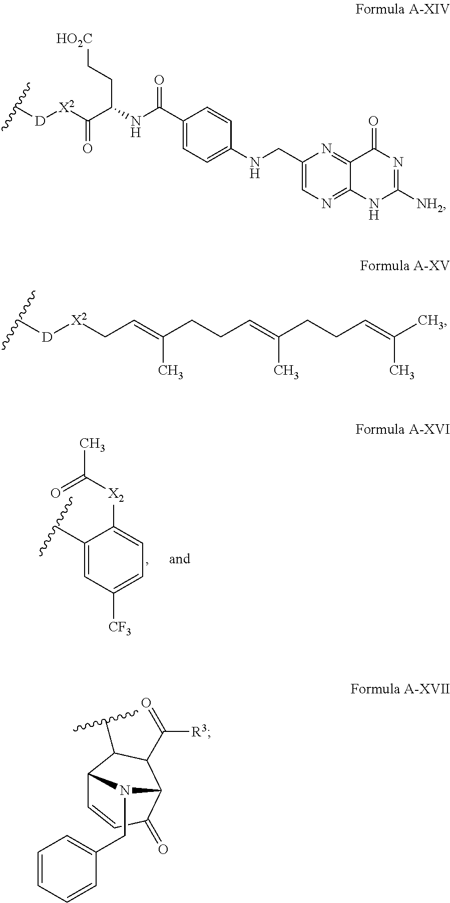

D is absent or

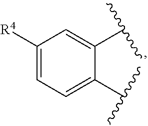

X² is selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B-Z;

R⁵ is selected from methyl and ethyl;

B is selected from:

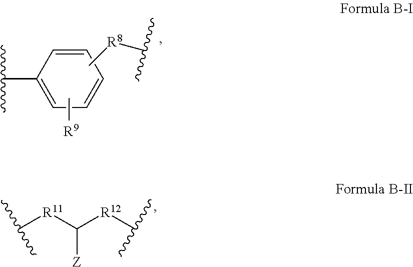

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;

Z is selected from:

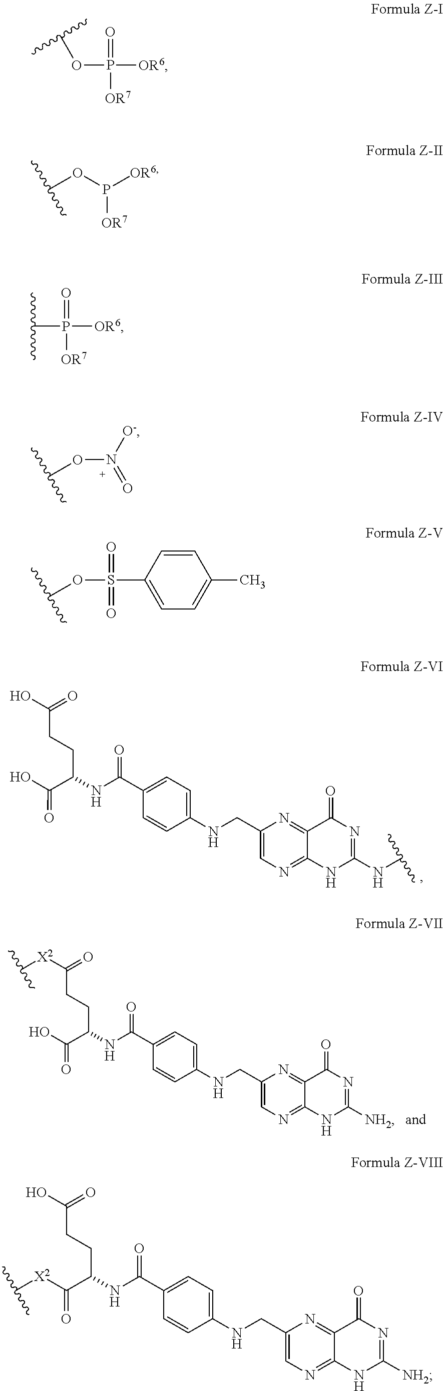

or B together with Z forms a structure:

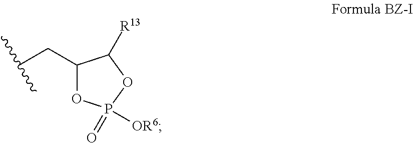

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

Exemplary compounds described herein also include compounds 93 to 108 specified above.

A further aspect of the present invention is directed to a pharmaceutical composition comprising a compound of the invention, as described generally herein, and a pharmaceutically acceptable excipient.

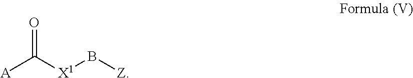

In Formula V: A is an optionally substituted aliphatic, heteroaliphatic, aromatic, heteroaromatic substituent or alkylaryl substituent having 1 to 100 carbon atoms;

X¹ is selected from —O—, —S—, and —NR⁵—;

R⁵ is selected from hydrogen and a C₁₋₆ alkyl;

B is an aliphatic, alicyclic, heteroaliphatic, heterocyclic, aryl, aralkyl, or heteroaromatic group optionally substituted with one or more R¹⁵ moieties,

each R¹⁴ is independently, selected from hydrogen, halogen, hydroxyl, alkoxyl, —CN; an optionally substituted aliphatic, alicyclic, heteroaliphatic, heterocyclic, aryl, aralkyl, heteroaromatic moiety; —OR^{R}, —S(═O)ₙR^{d}, —NR^{b}R^{c}, —C(═O)R^{a} and —C(═O)OR^{a}; n is 0-2; R^{a}, for each occurrence, is independently selected from hydrogen and an optionally substituted aliphatic, alicyclic, heteroaliphatic, heterocyclic, aryl, aralkyl, or a heteroaromatic moiety; each of R^{b} and R^{c}, for each occurrence, is independently selected from hydrogen; hydroxyl, SO₂R^{d}, and aliphatic, alicyclic, heteroaliphatic, heterocyclic, aryl, aralkyl, heteroaromatic or an acyl moiety; R^{d}, for each occurrence, is independently selected from hydrogen, —N(R^{e})₂, aliphatic, aryl and heteroaryl, R^{e}, for each occurrence, is independently hydrogen or aliphatic; and R^{R} is an optionally substituted aliphatic, alicyclic, heteroaliphatic, heterocyclic, aryl, aralkyl, heteroaromatic or acyl moiety;

Z is selected from:

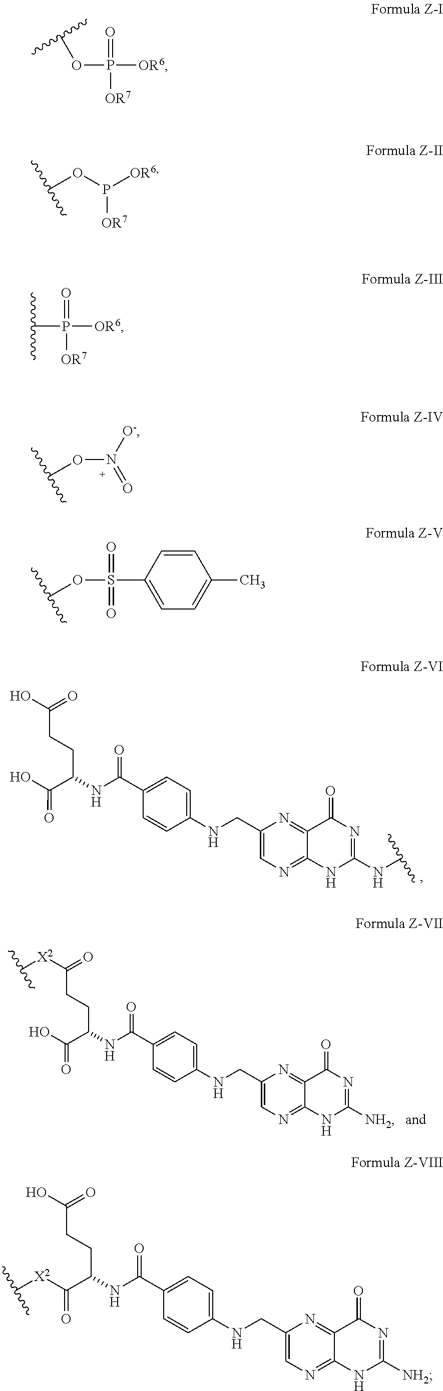

or B together with Z forms a structure:

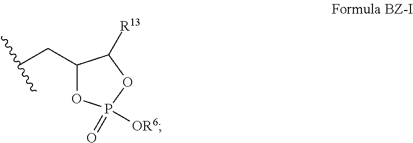

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue;

or a pharmaceutically acceptable salt thereof.

In a specific embodiment, the compound of Formula V is further described by Formula I, II, III, or IV or any specific compound described herein.

In another embodiment the compound of Formula V is a compound disclosed in U.S. Pat. No. 8,236,820, incorporated by reference.

Other embodiments and any of compounds 1-134, as well as exemplary methods for the synthesis of these compounds, are described herein.

### Utility and Administration

As discussed above, certain of the compounds represented by the invention can exhibit activity generally as inhibitors of pain, inflammation and/or cancer and precancerous conditions thereof. Thus, in certain embodiments, compounds of Formulas I-V (e.g., compounds 1-134) are useful for the treatment of any of a number of conditions or diseases in which inflammation, in particular chronic inflammation is the cause of or relates to the onset or continued occurrence of the disease or condition, such as but not limited to rheumatologic diseases such as rheumatoid arthritis and Sjogren's syndrome; cardiovascular diseases, for example, coronary artery disease, peripheral vascular disease and hypertension; neurodegenerative diseases, for example, Alzheimer's disease and its variants or cerebrovascular diseases; and autoimmune diseases such as lupus erythematosus; other conditions characterized by chronic inflammation of organs such as the lung, such as chronic bronchitis or the sinuses, such as chronic sinusitis. Moreover, compounds of the present invention are also useful for the treatment of cancers, in particular cancers of the breast, brain, and the digestive and respiratory systems.

Accordingly, in one aspect of the invention, methods for the treatment of inflammation-related disorders and/or cancer are provided comprising administering a therapeutically effective amount of a compound of any one of Formulas I-IV or any compound specified herein to a subject in need thereof. In certain embodiments, a method for the treatment of related disorders is provided comprising administering a therapeutically effective amount of a compound, or a pharmaceutical composition comprising an inventive compound to a subject in need thereof, in such amounts and for such time as is necessary to achieve the desired result.

The invention is also directed to the use of compounds of Formulas I-V (e.g., compounds 1-134) for the preparation of a medicament for administration to a human or animal patient in need thereof, to inhibit or block inflammation and/or inhibit the growth of cancer. Such compounds preferably are administered once an inflammation-related disease or an inflammatory condition that may predispose to disease or cancer has been diagnosed in the patient, optionally in combination with other anti-inflammation agents or other anti-cancer agents such as those that maintain therapeutic levels of the compounds within the body. Treatment may also be provided after other therapies have been tried and failed, and may be administered prophylactically.

As discussed above, compounds of Formulas I-V (e.g., compounds 1-134), as well as pharmaceutical compositions including these compounds, can be useful for the treatment and/or prevention of lung cancer and precancerous condition of the lung.

A compound or pharmaceutical composition containing the compound may be administered, for example, by the nasal or oral respiratory route. For example, compounds can be suspended or dissolved in an appropriate carrier and administered directly into the lungs using a nasal spray or inhalant. Alternatively, compounds and pharmaceutical compositions may be sprayed into the nasal cavity and absorbed through the nasal mucosa.

Importantly, direct inhalational administration of certain disclosed compounds into the lungs features several advantages over oral administration:
a) A lower amount of the compound is required for achieving the same therapeutic effect. This may be critical for expensive compounds.
b) Any potential undesired side effects of the compound are minimized.
c) Inactivation of the compound in vivo through first-pass metabolism, e.g. by non-specific esterases in the intestine and liver is substantially avoided.
d) Improved absorption using aerosol drug delivery.

Accordingly, in another aspect of the invention, methods for the treatment and/or prevention of lung and/or brain cancer and precancerous condition thereof are provided comprising administering a therapeutically effective amount of compounds of Formulas I-V (e.g., compounds 1-134) to a subject in need thereof by the respiratory route. In certain embodiments, the compounds and pharmaceutical compositions including these compounds are administered in such amounts and for such time as is necessary to achieve the desired result.

The invention is also directed to the use of compounds of Formulas I-V (e.g., compounds 1-134) for the preparation of a medicament for administration to a human or animal patient in need thereof for the treatment and/or prevention of lung and/or brain cancer and precancerous condition thereof. Such compounds are preferably administered once a precancerous condition of the lung or lung and/or brain cancer has been diagnosed in the patient, optionally in combination with anti-inflammation agents or other anticancer agents such as those that maintain therapeutic levels of the compounds within the body. Compounds also may be administered after other therapies have failed.

In another embodiment, compounds of Formulas I-V (e.g., compounds 1-134), as well as pharmaceutical compositions including these compounds, may be administered prophylactically for the purpose of prevention of lung and/or brain cancer. Thus, compounds and pharmaceutical compositions including these compounds may be administered to subjects having an increased risk of developing lung and/or brain cancer, for instance to smokers. Lung cancer is the culmination of a long transition of the tracheal epithelium from normal through various precancerous stages. Thus, administration of these compounds invention before or during this transitional period is simpler for the patient and is further cost-effective compared to current therapeutic modalities for already developed lung cancer.

In some embodiments of the invention, the pharmaceutical composition is administered to a human or animal in the form of an aerosol.

Yet in other embodiments of the invention, the pharmaceutical composition is administered to a human or animal in the form of a dry powder aerosol.

In a further embodiment of the invention the pharmaceutical composition is administered to a human or animal, preferably to a human, in combination with tobacco smoke. Thus, the corresponding envisioned mode of administration is for the compound of the invention to be inhaled at the same time when the smoker smokes.

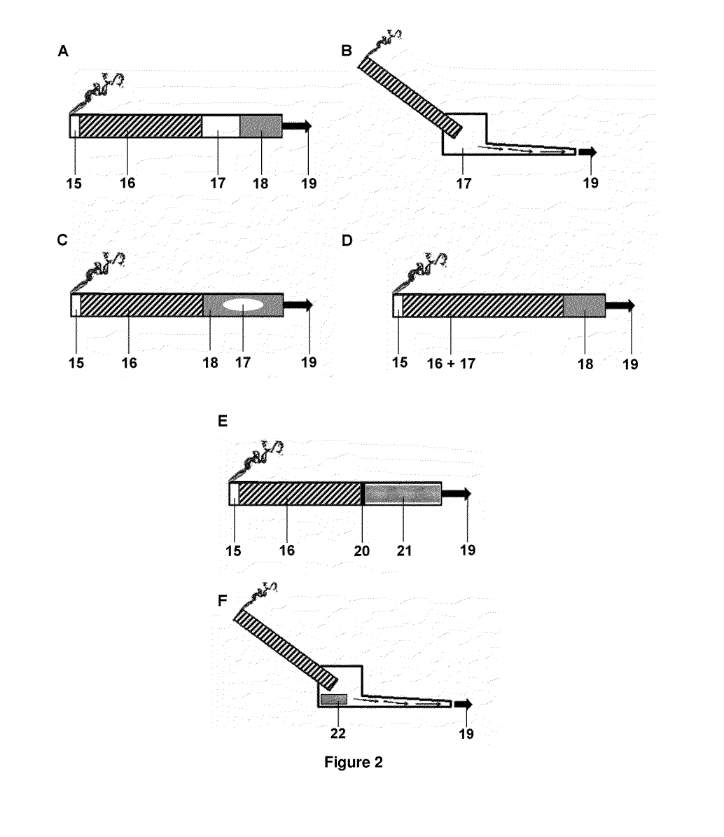

For this purpose, compounds of Formulas I-V (e.g., compounds 1-134) or a pharmaceutical composition thereof can be incorporated in a smoking device such as for instance a cigarette or a smoking pipe as shown in FIG. 2. In the embodiments illustrated by FIGS. 2A-2D, the number 17 indicates the location of the compound of the invention or a pharmaceutical composition thereof. In the smoking devices shown in FIG. 2E and FIG. 2F the compound or a pharmaceutical composition thereof is located in the cartridge 21 and in the additional unit 22 respectively.

In some embodiments of the invention, compounds of Formulas I-V (e.g., compounds 1-134) can be directly mixed with tobacco. In these embodiments, a vaporization of the compound takes place in the pyrolysis zone of the smoking device. These embodiments are particularly preferred for sufficiently volatile compounds of the invention. The vaporization of the compound can be additionally facilitated, when volatile solids such as menthol are used as carriers in the pharmaceutical composition.

The term “tobacco” as used herein relates to the leaf of a tobacco plant i.e. a plant of the genus Nicotiana, such as Nicotiana tabaccum. Tobacco leaves of several types may be employed. Suitable types of tobacco leaves include, but are not limited to, Brightleaf tobacco, Burley, Cavendish, Corojo, Criollo, Oriental tobacco, Perique, Shade tobacco, Thuoc lao, Type 22, White Burley, wild tobacco and Y1.

In other embodiments of the invention, the pharmaceutical composition that includes the compound is spatially separated from the tobacco.

Preferably, the aerosol particles comprise less than 10 wt.-% of degradation products formed by the compound. More preferred, the particles comprise less than 5 wt.-% of degradation products formed by the compound. Yet even more preferred, the particles comprise less than 1.0 wt.-%, for instance less than 0.5 wt.-% of degradation products formed by the compound.

Preferably, at least 50 wt.-% of the aerosol is amorphous in form, wherein crystalline forms make up less than 50 wt.-% of the total aerosol weight, regardless of the nature of individual particles. More preferred, at least 75 wt.-% of the aerosol is amorphous in form. Particularly preferred, at least 90 wt.-% the aerosol is amorphous in form.

Typically, the aerosol has an inhalable aerosol particle density greater than 10⁶ particles/ml. Preferably, the aerosol has an inhalable aerosol particle density greater than 10⁷ particles/ml or 10⁸ particles/ml.

Preferably, the aerosol particles have a mass median aerodynamic diameter of between 3 μm and 0.02 μm, more preferred between 2 μm and 0.05 μm, even more preferred between 1 μm and 0.1 μm, particularly preferred between 0.8 μm and 0.2 μm.

Particle size distribution of the aerosol can be determined using any suitable method in the art (e.g., cascade impaction). For example, an Andersen Eight Stage Nonviable Cascade Impactor (Andersen Instruments, Smyrna, Ga.) linked to a furnace tube by a mock throat (USP throat, Andersen Instruments, Smyrna, Ga.) is one system used for cascade impaction studies.

Inhalable aerosol mass density is determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the tidal volume of an inhaling patient.

Inhalable aerosol drug mass density is determined, for example, by delivering a drug-containing aerosol into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the tidal volume of an inhaling patient. The amount of the compound of the invention collected in the chamber is determined by extracting the chamber, conducting chromatographic analysis of the extract, for instance by using analytical HPLC, and comparing the results of the chromatographic analysis to those of a standard containing known amounts of the compound of the invention.

In certain embodiments, the uses and methods of the invention involve the administration of a therapeutically effective amount of the compound or a pharmaceutically acceptable derivative thereof to a subject (including, but not limited to a human or animal, including livestock, domesticated or zoo animals) in need thereof.

It will be appreciated that the compounds and compositions, according to the method of the present invention, may be administered using any amount and any route of administration effective for the treatment of conditions or diseases in which anti-inflammation, anti-cancer, analgesic, antipyretic or related activities have a therapeutically useful role. Thus, the expression “effective amount” as used herein, refers to a sufficient amount of agent to inhibit inflammation and to exhibit a therapeutic effect. The exact amount required will vary from subject to subject, depending on the species, age, and general condition of the subject, the severity of the infection, the particular therapeutic agent, its mode of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with appropriate pharmaceutically acceptable carriers in a desired dosage form, the pharmaceutical compositions of this invention can be administered to a human or animal subject orally, rectally, parenterally (intravascularly, intramuscularly, intraperitoneally, subcutaneously), intracisternally, intravaginally, topically in the form of a gel, cream, ointment, lotion or drops, bucally in the form of a gel or tablet, or the like, depending on the location and extent of the disease being treated. In certain embodiments, the compounds of the invention may be parenterally administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. In other embodiments, compounds of the invention may be administered orally or rectally at dosage levels of about 0.01 mg/kg to about 100 mg/kg, from about 0.05 mg/kg to about 50 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Furthermore, after pharmaceutical composition with appropriate pharmaceutically acceptable carriers in a desired dosage form, the pharmaceutical compositions of this invention can be administered to a human or animal subject. In certain embodiments, the compounds of the invention may be administered by inhalation at dosage levels of 0.001 mg/kg to 50 mg/kg, from 0.01 mg/kg to 25 mg/kg or from 0.1 mg/kg to 10 mg/kg of subject body weight per day, one or more times a day, to obtain the desired therapeutic effect. In other embodiments, compounds of the invention may be administered at dosage levels of 0.01 mg/kg to 100 mg/kg, from 0.05 mg/kg to 50 mg/kg or from 0.1 mg/kg to 10 mg/kg of subject body weight per day, one or more times a day, to obtain the desired therapeutic effect. It will also be appreciated that dosages smaller than 0.001 mg/kg or greater than 50 mg/kg (for example 50 mg/kg to 100 mg/kg) can be administered to a subject.

The inhalation of the compound of the invention can take place between one and seven times a day, for instance three times a day.

### Inhalation Devices

Contemplated for use in the practice of this invention are a wide range of mechanical devices designed for pulmonary delivery of the pharmaceutical compositions of the present invention, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp, Bedford, Mass.

Device for the nasal drug delivery are also known to persons skilled in the art and are commercially available, for instance, from Bespak (Bespak Europe Limited, United Kingdom).

In some other embodiments, the pharmaceutical composition of the present invention is directly heated, whereby the compound of the invention forms a vapor and subsequently condenses into an aerosol. Thus, an aerosol containing the compound of the invention is formed. Subsequently, the patient inhales this aerosol. Suitable devices are known in the prior art and are, for instance, described in US 2003/0000518.

In another embodiment, the compound of the invention or the pharmaceutical composition is dissolved in a solvent such as ethanol, glycerol, water, 1,3-propylene glycol or in a mixture of any of those. For instance, ethanol can be employed for this purpose.

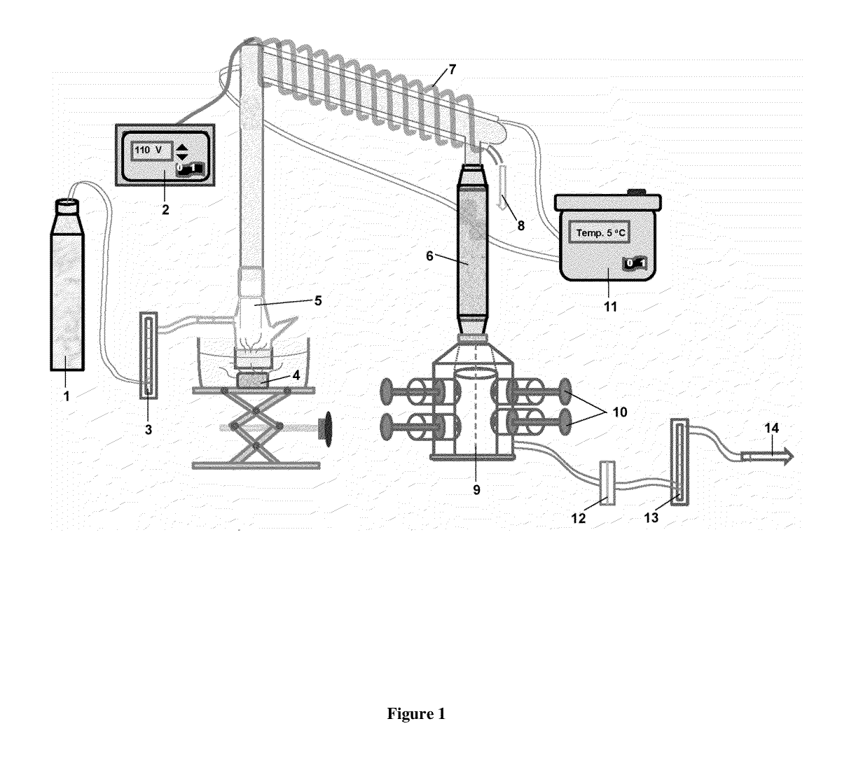

An example of inhalation device, in which the compound to be delivered is dissolved in a solvent is shown in FIG. 1. This exposure system can be employed for pre-clinical and clinical studies as well as for routine administration of the compound to the patients. Air flow in the device is controlled by two major elements:
a) an inlet air regulator equipped with a flow meter 3, which pushes air 1 into the device via the baffle 5; and
b) a vacuum pump 14 which draws air from the system. The air entering the vacuum pump 14 passes through a filter 12 and a flow meter 13.

The compound to be delivered is dissolved in ethanol and the solution in the baffle 5 is aerosolized with the ultrasonic atomizer 4. The aerosol formed passes through an ascending stainless steel column, followed by a reflux column which is maintained at a temperature gradient by a heating tape 7 (82° C.) and a condenser (5° C.) to condense and remove ethanol. The temperature of the heating tape 7 is adjusted by the voltage regulator 2. The aerosol of the compound of the invention exiting the reflux column then passes through a charcoal column 6 which serves to remove residual traces of ethanol from the aerosol before it enters the chamber 9. The patient can inhale the aerosol from air-tight tubes 10 for desired time intervals.

In another embodiment, the compound is administered in a so-called electronic cigarette. Such devices are known in the prior art and are, for instance, described in US 2006/0196518, US 2007/0267031 and Caponnetto et al (Journal of Medical Case Reports 5, 585, 2011). An electronic cigarette is primarily used for the administration of nicotine and, optionally, of flavors such as menthol. Incorporating the compound or the corresponding pharmaceutical composition in the nicotine cartridge thus allows efficient administration of the compound of the invention by the respiratory route. Advantageously, the cartridge containing the compound can be employed with a commercially available electronic cigarette.

Accordingly, another aspect of the present invention relates to a cartridge containing a compound of Formulas I-V (e.g., any one or more of compounds 1-134) or the pharmaceutical composition thereof for use in an electronic cigarette. Such cartridge can be primarily used by patients suffering from lung cancer or those with precancerous conditions in the lung.

Another envisioned mode of administration is for the compound to be inhaled at the same time that the smoker smokes. For this purpose, the compound or the pharmaceutical composition thereof can be for instance incorporated in a cigarette, a cigar (see FIG. 2A) or in a smoking device such as a smoking pipe (see FIG. 2B in the chamber of a smoking pipe) or in a water pipe etc.

FIG. 2A: the pharmaceutical composition 17 containing a compound to be delivered is incorporated into the cigarette containing tobacco 16 and, optionally, having a filter 18. Tobacco smoke coming from the pyrolysis zone 15 causes volatilization of the compound 17. In order to improve volatilization, the compound can be formulated with a volatile solid such as menthol. The tobacco smoke 19 containing the compound enters the mouth and the lungs of the smoker.

FIG. 2B: the pharmaceutical composition 17 is incorporated into a smoking pipe. Alternatively, another smoking device such as water pipe can be employed. The volatilization of the pharmaceutical composition 17 can be additionally facilitated by an external heating, for instance, by using an electric heating element.

In FIG. 2C a further embodiment of the present invention is shown. A compound is administered in a so-called “cigarette with menthol capsules.” The pharmaceutical composition 17 is incorporated in a menthol capsule, which, in turn, is located in the filter 18. Cigarettes with menthol capsules are known in the prior art and are, for instance, described in US 2009/0277465. The compound of the invention or the pharmaceutical composition thereof is incorporated into the menthol capsule and is volatilized during the smoking process. Thus, this embodiment is particularly suited for smokers and aims to prevent lung cancer and/or precancerous conditions in the lung.

In a further embodiment shown in FIG. 2D, the pharmaceutical composition 17 is directly mixed with tobacco. Thus, volatilization the compound occurs primarily in the pyrolysis zone 15 of the cigarette and the tobacco smoke 19 containing the compound of the invention enters the mouth and the lungs of the smoker. In this embodiment, the filter 18 is optional. This embodiment is particularly useful, if the compound of the invention is sufficiently volatile.

A further embodiment is shown in FIG. 2E. The pharmaceutical composition 17 (not shown) is incorporated in an electronic cigarette cartridge 21. Valve 20 prevents the entry of the aerosol and solvent vapor emitted by the cartridge 21 into the tobacco section 16. In this embodiment, tobacco smoke formed in the pyrolysis zone 15 enters the section containing the electronic cigarette cartridge 21 via the valve 20. Thus, the aerosol emitted by the electronic cigarette cartridge 21 is mixed with the tobacco smoke and the resulting mixture 19 is subsequently inhaled by the smoker.

FIG. 2F: a further embodiment is shown. The anti-cancer agent or a pharmaceutical composition thereof 17 (not shown) is incorporated in an additional unit 22 which may be an atomizer or cartonizer or similar device that renders the anti-cancer agent suitable for inhalation. Having an appropriate valve or other mechanism(s), smoke and inhalable agent may be mixed to simultaneously deliver smoke and anti-cancer agent to the mouth and ultimately the lungs of the smoker.

In all embodiments shown in FIGS. 2A-2F during inhalation of tobacco smoke, the smoker also inhales the desired compound. In order to facilitate volatilization of the compound of the invention, it can be formulated in a dry powder aerosol composition such as the one described by C. Plumley, et al. (Int. J. Pharm. 369, (1-2), pages 136-143, 2009) or in a pharmaceutical composition containing volatile solids such as menthol. Alternatively, a neat compound of the invention can be used instead of the pharmaceutical composition thereof.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, for instance if the patient suffers from Alzheimer's disease, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Pharmaceutical Compositions

As discussed above, this invention features compounds for use in the treatment and prevention of lung and/or brain cancer and precancerous conditions thereof, wherein said compounds are administered to a human or animal by the respiratory route. The term “respiratory route” as used herein refers to both nasal and pulmonary respiratory routes.

As discussed above, this invention features compounds that have biological properties and pharmacological activity useful for the treatment of any of a number of conditions or diseases generally characterized by abnormal inflammation, or prophylaxis in instances wherein a risk of appearance of such conditions or diseases is present as well as for the treatment and/or prevention of cancer. Moreover, certain compounds known in the art have been newly identified as having activity likewise useful in the prophylaxis or treatment of abnormal inflammation and cancers, and the invention is also directed to anti-inflammation and anti-cancer compositions comprising such compounds.

Accordingly, in another aspect of the present invention, pharmaceutical compositions are provided, which comprise any one of the compounds described herein (e.g., a compound of Formulas I-V or one of compounds 1-134), or a pharmaceutically acceptable salt or other pharmaceutically acceptable derivative thereof, and optionally comprise a pharmaceutically acceptable carrier. In certain embodiments, these compositions optionally further comprise one or more additional therapeutic agents. Alternatively, any of the above compounds may be administered to a patient in need thereof in combination with the administration of one or more other therapeutic agents. For example, additional co-administered therapeutic agents or included in a pharmaceutical composition with the aforementioned compound may be an approved anti-inflammation agent, or it may be any one of a number of agents undergoing approval in the Food and Drug Administration that ultimately obtain approval for the treatment of any disorder related to inflammation. Such additional therapeutic agents may also be provided to promote the targeting of the compound to the desired site of treatment, or may increase their stability, increase their plasma half-life, and further improve their biodistribution and pharmacokinetics. It will also be appreciated that certain of the compounds described herein can exist in free form for treatment, or where appropriate, as a pharmaceutically acceptable derivative thereof. According to the present invention, a pharmaceutically acceptable derivative includes, but is not limited to, pharmaceutically acceptable salts, esters, salts or cocrystals of such esters, or a pro-drug or other adduct or derivative of a compound described herein which upon administration to a patient in need is capable of providing, directly or indirectly, a compound as otherwise described herein, or a metabolite or residue thereof.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Administration by the nasal respiratory route includes nasal administration, and nose to brain delivery whereby the composition of the present invention is sprayed into the nasal cavity and delivered to the brain via the olfactory and trigeminal neural pathways. Nasal drug delivery is known to a person skilled in the art and is, for instance, described in L. Ilium (J. Control. Release 87 (2003), pp. 187-198). Administration by nasal respiratory route and nose to brain delivery is particularly suitable for the treatment of brain cancer and the corresponding precancerous conditions.

Preferably, the permeability of the nasal mucosa to the compounds described herein is high, and subsequently, their bioavailability upon nasal administration is more than 60%, preferably more than 70% and even more preferred more than 80%.

When the composition is administered by the nasal respiratory route, more than 50 wt.-%, preferably more than 60 wt.-% and particularly preferred more than 70 wt.-% of the compound is absorbed through the nasal mucosa and enters the systemic circulation of the patient. Thus, this embodiment of the present invention allows a rapid and effective administration of the compound. Furthermore, if the aerosol particles have mass median aerodynamic diameter of less than 10 μm, up to 40 wt.-%, preferably up to 50 wt.-% and more preferred up to 60 wt.-% of the compound of the invention is delivered to the lungs of the patient. Accordingly, the compound is delivered to the lung cancer of the patient both locally and systemically.

The composition for nasal administration may be an aqueous solution designed to be administered to the nasal passages in form of drops or sprays. Preferably, this composition is isotonic to nasal secretions and slightly buffered to maintain a pH of 5.5 to 6.5. Antimicrobial agents and/or preservatives may be also present in this composition.

In another embodiment of the invention, the composition is administered by the oral respiratory route.

For administration by the respiratory route, the compounds can be delivered in the form of an aerosol spray from a pressurized container or dispenser, which contains a suitable propellant, e.g. hydrofluoroalkanes, chlorofluorocarbons, carbon dioxide, or a nebulizer. In the case of a pressurized aerosol, the dosage unit may be determined by providing a valve to deliver a metered amount. Capsules and cartridges of e.g. gelatine for use in an inhaler or insufflator may be formulated containing a powder mix of the compound and a suitable pharmaceutically acceptable carrier.

Administration by the respiratory route usually requires the use of pharmaceutical compositions suitable for the dispensing of the compounds. Typically, each pharmaceutical composition is specific to the type of device employed and may involve the use of an appropriate propellant material, in addition to the usual diluents, adjuvants and/or carriers. Also, the use of liposomes, microcapsules or microspheres, inclusion complexes, or other types of carriers is contemplated. The compounds of the invention may be prepared in different pharmaceutical compositions depending on their physical and chemical properties or the type of device employed.

Pharmaceutical composition suitable for use with a nebulizer, either jet or ultrasonic, will typically comprise the compound dissolved in a solvent at a concentration of about 0.1 to 25 mg of the compound of the invention per 1 ml of solution. The pharmaceutical composition may also include a buffer, for instance, an amino acid, and a simple sugar (e.g. for compound of the invention stabilization and regulation of osmotic pressure). The solvent in the pharmaceutical composition may be selected from the group consisting of water, ethanol, 1,3-propylene glycol, glycerol or a mixture of any of those. Nebulized pharmaceutical compositions may also contain a surfactant, to reduce or prevent surface induced aggregation of the compound caused by atomization of the solution in forming the aerosol.

Pharmaceutical compositions for use with a metered-dose inhaler device generally comprise a finely divided powder containing one ore more of the described compounds (or a pharmaceutically acceptable derivative thereof) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Pharmaceutical compositions for dispensing from a powder inhaler device will comprise a finely divided dry powder containing the compound and may also include a bulking agent, such as lactose, sorbitol, sucrose, or mannitol in amounts, which facilitate dispersal of the powder from the device, e.g. 50 to 90% by weight of the formulation. The compound should most advantageously be prepared in a particulate form with an average particle size of less than 10 μm, preferably less than 5 μm and more preferred less than 1 μm, for effective delivery to the distal lung.

In another aspect of the present invention, pharmaceutical compositions are provided, which comprise a compound of Formulas I-V (e.g., one or more of compounds 1-134), or a pharmaceutically acceptable salt or other pharmaceutically acceptable derivative thereof, and optionally comprise a pharmaceutically acceptable carrier. In certain embodiments, these compositions optionally further comprise one or more additional therapeutic agents. Alternatively, the compounds of this invention may be administered to a patient in need thereof in combination with the administration of one or more other therapeutic agents. For example, additional co-administered therapeutic agents or included in a pharmaceutical composition with a compound of this invention may be an approved anti-inflammation or analgesic agent, or it may be any one of a number of agents undergoing approval in the Food and Drug Administration that ultimately obtain approval for the treatment of any disorder related to inflammation and pain. Such additional therapeutic agents may also be provided to promote the targeting of the compounds of the invention to the desired site of treatment, or may increase their stability, increase their plasma half-life, and further improve their biodistribution and pharmacokinetics. It will also be appreciated that certain of the compounds of present invention can exist in a free form for treatment, or where appropriate, as a pharmaceutically acceptable derivative thereof. According to the present invention, a pharmaceutically acceptable derivative includes, but is not limited to, pharmaceutically acceptable salts, esters, salts or cocrystals of such esters, or a pro-drug or other adduct or derivative of a compound of this invention which upon administration to a patient in need is capable of providing, directly or indirectly, a compound as otherwise described herein, or a metabolite or residue thereof.

Liquid dosage forms for oral administration include, but are not limited to, pharmaceutically acceptable, emulsion preconcentrates the so-called self-emulsifying drug delivery systems (SEDDS), emulsions, microemulsions, nanoemulsions, solutions, suspensions, syrups and elixirs. In addition to the active compounds, the liquid dosage forms may contain inert diluents commonly used in the art such as, for example, water or other solvents, solubilizing agents, oils and emulsifiers. Suitable solvents include ethyl alcohol, isopropyl alcohol, ethyl carbonate, ethyl acetate, benzyl alcohol, benzyl benzoate, propylene glycol, 1,3-butylene glycol, dimethylformamide, glycerol, tetrahydrofurfuryl alcohol and polyethylene glycols. Oil components include soybean, cottonseed, groundnut (peanut), corn, germ, olive, castor, almond, sesame and fish oil,and mixtures thereof. Surfactants suitable for the compositions of the present invention, include mono- and/or diglycerides of fatty acids and their acetic, succinic, lactic, citric and/or tartaric esters, propylene glycol fatty acid esters, mixtures of propylene glycol esters and glycerol esters, polyethoxylated fatty acids, PEG-fatty acid diesters, PEG-fatty acid mono- and di-ester mixtures, polyethylene glycol and glycerol fatty acid esters, alcohol-oil trans-esterification products, polyglycerized fatty acids, polyethylene glycol sorbitan fatty acid esters, polyethylene glycol alkyl ethers, sorbitan fatty acid esters, lower alcohol fatty acid esters, sugar esters, vitamin E esters, such as α-tocopherol-polyethylene-glycol-1000-succinate, poloxyethylene-polyoxypropylene block copolymers, known as Pluronics® also known as Poloxamers, lecithin, C6-C22 fatty acids and salts and mixtures thereof. Besides inert diluents, the oral compositions can also include adjuvants such as wetting agents, emulsifying and suspending agents, sweetening, flavoring, and perfuming agents.

The oral liquid compositions of the present invention can be filled into hard or soft gelatin capsule or as bulk oral solutions in a bottle. These dosage forms can be manufactured by well established methods that are known in the art. The liquid-filled capsules can be further coated with enteric polymers for releasing the active in the small intestine or colon.

Injectable preparations, for example, sterile injectable aqueous or oleaginous suspensions and dispersions may be formulated according to the known art using suitable dispersing or wetting agents and suspending agents. The sterile injectable preparation may be a solution, suspension or emulsion in a nontoxic parenterally acceptable diluent or solvent. Among the acceptable aqueous vehicles and solvents that may be employed are water, Ringer's solution, U.S.P. and isotonic sodium chloride solution. In addition, vegetable oils phospholipids and surfactants form the list provided above and approved for parenteral drug administration are conventionally employed as a solvents, suspending or dispersing agents.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, preferably into liposomes, which are more biocompatible.

Compositions to deliver the agent directly to the colon—for example, tablets or capsules incorporating enteric coating pH-sensitive polymers, such as those available by the trade name Eudragit® and/or polysaccharides, such as pectin, from which the active agent is released into the colon by a pH-dependent mechanism and/or through degradation by the bacteria present in the colon or by other mechanism, ensuring exclusive or predominant colonic delivery of said compound. Other means for colonic delivery include suppositories and enemas.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include but are not limited to capsules, tablets, pills, pellets, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, pills and pellets, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, pellets, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The compounds of the invention are also suitable for incorporation into nanoparticulate systems such as liposomes, polymeric nanoparticles, polymeric micelles, lipid nanoparticles, micro- and nano-emulsions, nanogels, liposomes being particularly preferred. The corresponding nanoparticulate systems are known in the prior art and are, for instance, described in the review by Wu and Mansour (X. Wu and H. M Mansour, Invited Paper. International Journal of Nanotechnology: Special Issue-Nanopharmaceuticals, 2011, 8, 1/2, 115-145).

Nanoparticulate systems typically have an average particle size ranging from 1 to 1000 nm, preferably from 50 to 500 nm. The term “liposomes” as used herein refers to phospholipid vesicles with average particle size ranging from 50 to 1000 nm, which are formed by one or several lipid bilayers with an aqueous phase both inside and between the bilayers. The term “polymeric nanoparticles” refers to solid colloidal particles comprising polymeric materials. The average particle size of polymeric nanoparticles ranges from 30 to 300 nm.

In particular, the compounds of the invention are highly suitable for incorporation into liposomes. The resulting compositions are particularly useful for the treatment and/or prevention of cancers such as lung cancer and colon cancer. Preferred liposome compositions are those which in addition to other phospholipids, incorporate pegylated phospholipids, such as DSPE-PEG2000, and exhibit long circulation times by avoiding uptake and clearance by the reticuloendothelial system (RES) and thus, are able to reach and treat solid tumors in the body.

Polymeric micelles are particles formed through the self-assembly of amphiphilic block copolymers containing hydrophobic and hydrophilic blocks.

Lipid nanoparticles may be in the form of solid lipid nanoparticles, nanostructured lipid carriers or lipid drug conjugates. Microemulsions are typically characterized by the average internal globule size of less than 150 nm. Microemulsions require a surfactant concentration of at least 10 wt.-%, preferably of at least 50 wt.-% and more preferred of at least 20 wt.-%, based on the weight of the composition.

The term “nanogel” refers to aqueous dispersions of hydrogel particles formed by physically or chemically cross-linked polymer networks of nanoscale size. Nanogels can be prepared by a variety of methods such as self-assembly of polymers, polymerization of monomers, cross-linking of preformed polymers or template-assisted nanofabrication.

Use of nanoparticulate systems according to the present invention features sustained-release of the compound of the invention in the lung tissue, resulting in a reduction of dosing frequency and improved patient compliance and further enabling uniformity of drug dose distribution among the alveoli. Moreover, by formulating the compounds of the invention as in nanoparticulate systems, one can achieve a dose that is higher than that of other pharmaceutical compositions, which are limited by the solubility volatibility of the compound of the invention. Nanoparticles can be internalised by a variety of cell types and becide macrophages, other cells like cancer cells and epithelial cells are also able to take up nanoparticles. Therefore, usage of nanoparticulate systems for delivering the compounds of the invention is highly advantageous for the treatment and prevention of lung cancer.

Nanoparticulate formulations can further be advantageously used for the nasal delivery of the compounds of the invention. In this embodiment, multiple-unit mucoadhesive nanoparticles are preferably used in order to prolong the contact of the compound of the invention with the nasal mucosa.

The resulting compositions can be advantageously employed for administration by the respiratory route. Preferred liposome compositions are those which in addition to other phospholipids, incorporate pegylated phospholipids, such as DSPE-PEG2000, and exhibit long circulation times by avoiding uptake and clearance by the reticuloendothelial system (RES) and thus, are able to reach and treat lung cancer tumors.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include but are not limited to polymeric substances and waxes.

The present invention encompasses pharmaceutically acceptable topical formulations of inventive compounds of the invention. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, polyhydric alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16^{th} Edition, 1980 and 17^{th} Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, poloxamers and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in Formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. P., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. Gel compositions for applying the active compounds of the present invention to the skin, particularly those incorporating Pluronic® surfactants also known as poloxamers, such as Pluronic P123, are preferred. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

The hydrogel of the present invention preferably comprises at least one poloxamer. It is further preferred that the hydrogel comprises at least one permeation enhancer. The permeation enhancer is preferably selected from the group consisting of fatty acids, ethanol, non-ionic surfactants such as polyoxyethylene fatty acid esters, and lecithin, more preferably the permeation enhancer is oleic acid, lauric acid, ethyl myristate, isopropyl myristate, propylene glycol, isopropyl alcohol, a low molecular weight polyethylene glycol, Polysorbate 80, Sorbitan stearate or lecithin. Most preferably, the hydrogel of the present invention comprises a poloxamer and oleic acid.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent suitable for treating benign skin conditions), or they may achieve different effects (e.g. control of any adverse effects).

In certain embodiments, the pharmaceutical compositions of the present invention further comprise one or more additional active ingredients. The additional active ingredient may be a known agent for the treatment of non-cancerous skin and mucous membrane conditions, such as podophyllin resin, imiquimod, trichloroacetic acid, and/or podophyllotoxin. Preferably, the combination therapy comprises administration of a compound of Formula V and α-difluoromethylornithine (DFMO). For example, the combination therapy comprises administering any one of compounds 1 to 134 in combination with DFMO. In another embodiment, the combination therapy comprises administering a compound of Formula V, wherein A is a sulindac derivative, in combination with DFMO.

In some embodiments, the pharmaceutical composition may further comprise an additional compound having anticancer activity. The additional compound having anticancer activity can be selected from the group of compounds such as chemotherapeutic and cytotoxic agents, differentiation-inducing agents (e.g. retinoic acid, vitamin D, cytokines), hormonal agents, immunological agents and anti-angiogenic agents. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having anticancer activity can be found in L. Brunton, B. Chabner and B. Knollman (eds). Goodman and Gilman's The Pharmacological Basis of Therapeutics, Twelfth Edition, 2011, McGraw Hill Companies, New York, N.Y.

In a preferred embodiment, the additional compound having anticancer activity is a tyrosine kinase inhibitor (TKI). A TKI inhibits the tyrosine kinase activity of at least one tyrosine kinase. The inhibition may be reversible or irreversible. TKIs include, but are not limited to, agents such as imatinib, dasatinib, nilotinib, gefitinib, erlotinib, lapatinib, sunitinib, sorafenib and pazopanib. Various TKIs are, for instance, described in Hartmann et al. (J. Th. Hartman et al. Cur. Drug Metab, 2009, 10, pp. 470-481).

In another embodiment, the additional compound having anticancer activity is a compound with oxidative stress-inducing ability. These compounds increase the oxidative stress of cancer cells by inhibiting the mechanisms that cancer cells utilize to compensate for reactive oxygen species (ROS) and/or activating cellular signaling pathways that lead to immunocytotoxicity. Examples of the anticancer drug include platinum formulation such as cis-platin, carboplatin, and oxaliplatin, thiostrepton, cyclophosphamide, fluorouracil, etoposide, doxorubicin, bleomycin, and mitomycin. The term “reactive oxygen species” relates to highly reactive metabolites of molecular oxygen, which are generated in a tissue environment. ROS can be free radicals, ions or molecules. Examples of ROS include, but are not limited to, superoxide ion radical (O₂⁻), hydroxyl radical (OH.), peroxide (ROO.), alkoxyl radicals (RO.), hydrogen peroxide (H₂O₂), organic peroxide (ROOR′), ozone (O₃), singlet oxygen (¹O₂), etc.

Additional compounds having anticancer activity are preferably difluoromethylornithine, erlotinib and thiostrepton.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with an anti-inflammation or anticancer agent), or they may achieve different effects (e.g., control of any adverse effects).

In certain embodiments, the pharmaceutical compositions of the present invention further comprise one or more additional therapeutically active ingredients (e.g., anti-inflammatory and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications and anti-sickness drugs.

In certain embodiments the compounds of the present invention can be covalently or non-covalently bound to for example polyethylene glycol or other similar molecules to make them suitable for administration to the patient either in one of the forms described above or using nanodevices. In a preferred embodiment of the present invention, the compounds can be formulated into nanoparticles to optimize their delivery, intracellular targeting and therapeutic effect. Particularly preferred nanoparticulate compositions of the compounds are liposomes, solid lipid nanoparticles and polymeric micelles, particularly PEO-b-PLA [poly(ethylene oxide)-b-poly(lactid acid) micelles and dendrimers.

The following examples are put forth so as to provide those of ordinary skill in the art with a complete disclosure and description of how the methods and compounds claimed herein are performed, made, and evaluated, and are intended to be purely exemplary of the invention and are not intended to be limiting.

### EXAMPLES

### Materials and Methods

All reagents and solvents were ACS grade. All experiments involving moisture- or air-sensitive compounds were conducted under dry nitrogen. The starting materials and reagents, unless otherwise specified, were of the best grade commercially available (Aldrich, Fluka) and used without further purification. After purification, all new products showed a single spot on TLC analysis in two different solvent systems. All experiments were performed under atmospheric pressure of 100.3±5 kPa and room temperature unless stated otherwise. The term “room temperature” refers to a temperature of 20±2° C.

### Example 1

### Phosphoric acid diethyl ester 4-[2-(4-isobutyl-phenyl)-propionylamino]-butyl ester (phospho-ibuprofen amide, 105)

The title compound 105 was synthesized as shown in Scheme 1 below.

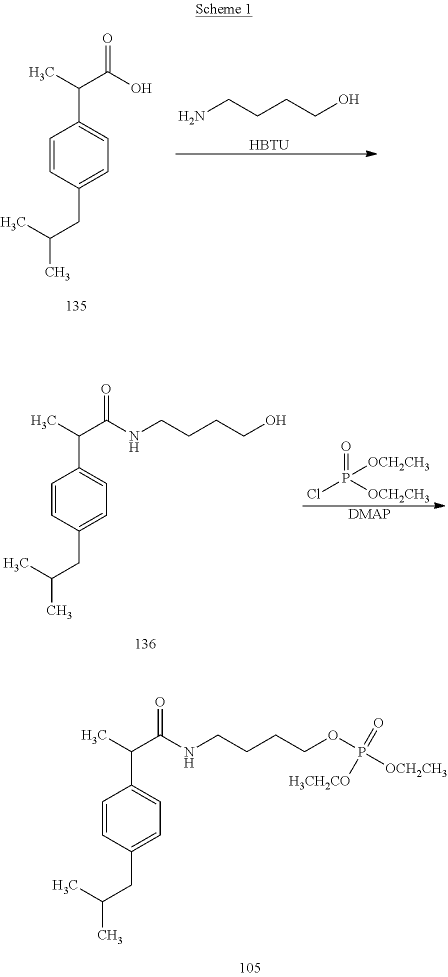

### Step 1.1 Synthesis of N-(4-Hydroxy-butyl)-2-(4-isobutyl-phenyl)-propionamide (136)

Ibuprofen (135) (0.228 g, 1 mmol), 4-amino-1-butanol (0.138 ml, 1.5 mmol) and O-(Benzotriazol-1-yl)-N,N,N′,N′-tetramethyluronium hexafluorophosphate (HBTU) (0.57 g, 1.5 mmol) were dissolved in 5 ml of N,N-dimethylformamide (DMF) containing N,N-diisopropylethylamine (DIPEA) (0.17 ml, 1 mmol). The reaction mixture was stirred at room temperature for 4 h. The reaction was monitored by TLC. The resulting reaction mixture was dissolved in ethyl acetate, and then washed with 1M HCl, saturated aqueous NaHCO₃ solution, distilled water, brine and dried over sodium sulfate (Na₂SO₄). After the solvent was removed, the crude product was purified by flash column chromatography to give 136 as a white solid in 95% yield.

### Step 1.2 Synthesis of phosphoric acid diethyl ester 4-[2-(4-isobutyl-phenyl)-propionylamino]-butyl ester (105)

Under nitrogen, diethyl chlorophosphate (0.43 g, 1.25 mmol) was added drop-wise to a solution of alcohol 136 (0.299 g, 1 mmol) in dichloromethane (10 ml) containing DIPEA (0.17 ml, 1 mmol), and 4-(dimethylamino)pyridine (DMAP) (6 mg, 0.05 mmol). The reaction mixture was stirred overnight and monitored by TLC. The obtained reaction solution was washed with water (2×25 ml), dried over anhydrous Na₂SO₄, filtered and concentrated. The crude residue was purified by column chromatography using n-hexane:ethyl acetate (60:40) as eluent. The pure fractions were combined and evaporated to give a slightly yellow liquid 1 in 85% yield.

### Biodistribution of Phospho-Ibuprofen Amide 105

Methods: Phospho-ibuprofen amide 105 was formulated in liposomes following the standard protocols described by Mattheolabakis et al. (G. Mattheolabakis, T. Nie, P. P. Constantinides, B. Rigas, Pharm. Res. 2012; 29:1435-43) and administered intravenously to mice as a single 200 mg/kg i.v. dose. After 1 h, blood and all major organs were collected and drug concentration was determined in them following already published methods (T. Nie et al. Br J Pharmacol. 2012; 166(3):991-1001).

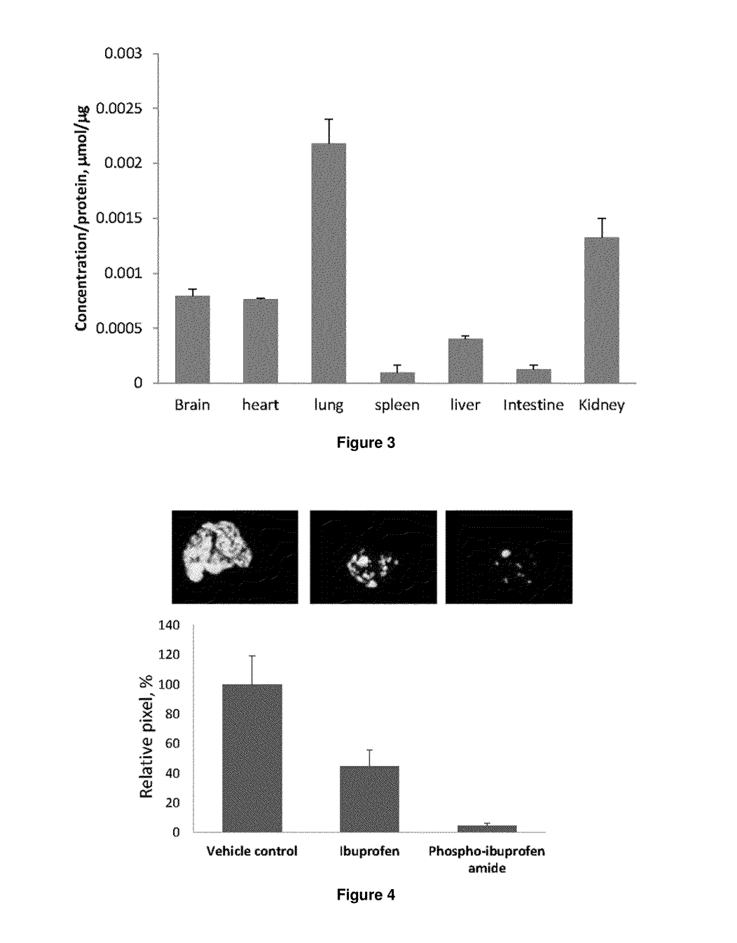

Results: As shown in FIG. 3, liposomal phospho-ibuprofen amide 105 preferentially accumulated in lungs.

### Efficacy of Phospho-Ibuprofen Amide 105: Inhibition of Lung Cancer

Methods: Female Ncr nude mice (6-7 weeks old) were injected i.v. (via their tail vein) with 6×10⁶ A549 human non-small lung cancer cells engineered to stably express green fluorescence protein. These cells were transplanted to the lungs (orthotopic lung tumor model). Three groups (n=6) of such mice were treated with a) liposomal phospho-ibuprofen amide 1 200 mg/kg, or b) ibuprofen (125) 200 mg/kg or c) vehicle once a week for 8 weeks. Mouse fluorescence was monitored using an in vivo imaging system (Maestro, Wobum, Mass.). Relative green fluorescence intensity units (from 7.5×10⁴ to 3.0×10⁵) were used as a marker for tumor initiation in the lungs. Day 0 was designated as initial detection of disease and the day before start of treatment. At the end of the study, animals were sacrificed and their tumors were removed, weighed and imaged.

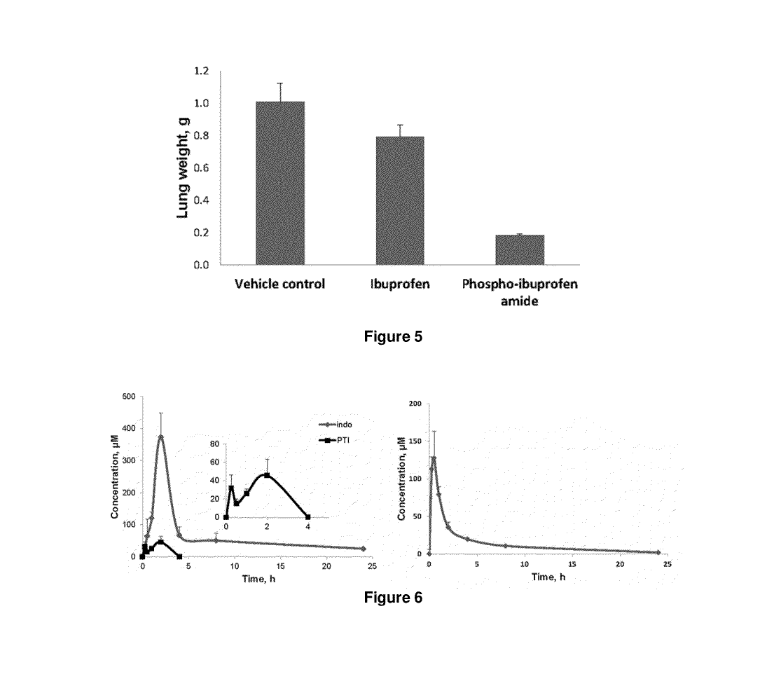

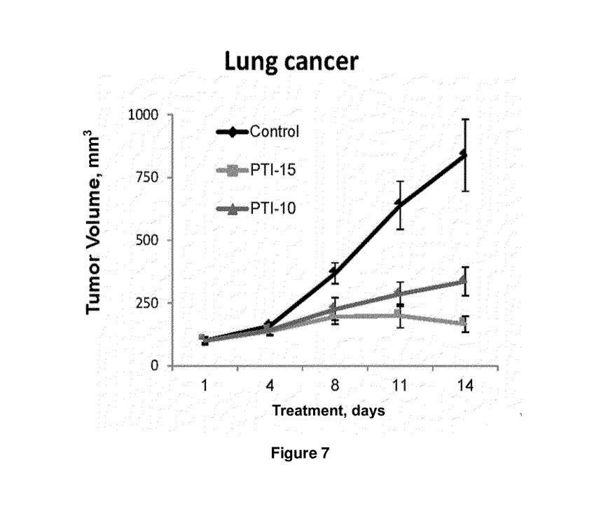

Results: FIG. 4 shows, in addition to representative fluorescence images of lungs from control (left), ibuprofen (center) and phospho-ibuprofen amide 105 (left) treated mice, the amount of lung tumor per group (based on fluorescence intensity). FIG. 5 depicts the lung weight of the same groups of animals. Values (% control) are mean±SEM.

Phospho-ibuprofen amide 105 essentially eliminated lung cancer, reducing it, by 95% based on fluorescence and by 80% base on lung tumor weight. In contrast, ibuprofen reduced tumor fluorescence by 57% and lung weight by 19%. The differences between phospho-ibuprofen amide 105 and ibuprofen were statistically significant (p<0.01).These findings underscore the efficacy of the compounds of the invention.

### Example 2

### Cellular Uptake of Ibuprofen, Phospho-Ibuprofen 132 and Phospho-Ibuprofen 137

### Test Compounds

Phospho-ibuprofen derivatives 132 and 137 as shown below and ibuprofen.

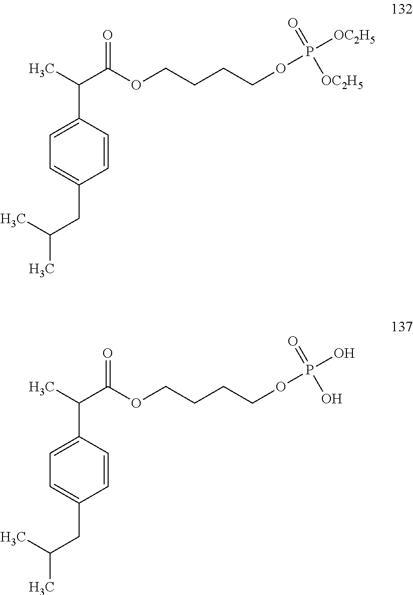 [SMILES: CCOP(=O)(OCC)OCCCCOC(=O)C(C)c1ccc(CC(C)C)cc1 ; CC(C)Cc1ccc(C(C)C(=O)OCCCCOP(=O)(O)O)cc1]

### Methods

A431 cells were seeded into 6-well culture plates (5×10⁵ per well). After overnight incubation, the cells were incubated with 100 μM ibuprofen, PI-phosphate 137 and PI-diethylphosphate 132 for 1 h. The media were removed and the monolayers were washed three times with PBS (1% BSA). Finally, the cells were collected in 200 μl PBS, after which 600 μl of acetonitrile was added to extract intracellular drugs. The intracellular levels were determined by HPLC analysis. The compounds evaluated have equivalent molar absorptivity.

### Results

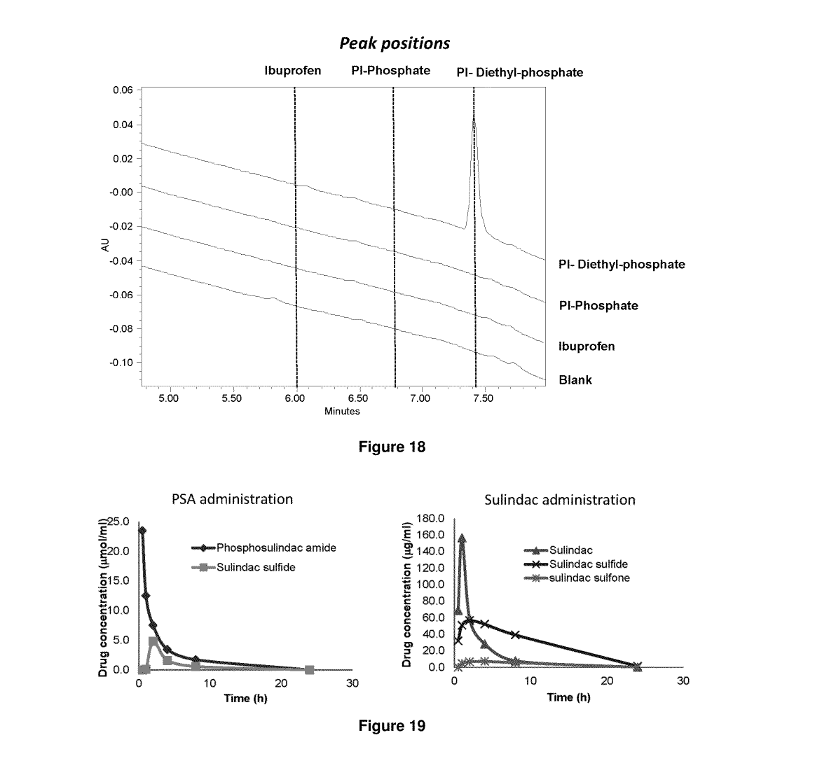

To evaluate the relative cellular uptake of ibuprofen, PI-phosphate 137 and PI-diethylphosphate 132, we incubated these compounds (100 μM for 1 h) with A431 skin cancer cells, and measured their cellular content by HPLC. As shown in FIG. 18, we did not detect significant accumulation of either ibuprofen or PI-phosphate 137 in A431 cells after 1 h incubation (limit of detection: 2.5 pmol). On the other hand, we found a high level of PI-diethylphosphate 123 in the cellular extract (750 pmol), representing at least 300-fold increase over the other two compounds.

The first HPLC chromatogram illustrates that after one hour incubation a significant amount of phospho-ibuprofen-diethyl phosphate 132 (retention time: 7.43 minutes) was accumulated in the cells. Importantly, neither ibuprofen (retention time: 6.00 minutes) nor phospho-ibuprofen-phosphate 137 (retention time: 6.78 minutes), which could potentially result from the intracellular hydrolysis of phospho-ibuprofen-diethyl phosphate where detected in the cellular extract.

When A431 cells were exposed to phospho-ibuprofen phosphate 137 or ibuprofen the cellular uptake of these compounds was below the limit of detection. Thus, phospho-ibuprofen-diethyl phosphate 132 is taken up by human cells A431 to a significantly higher extent compared to phospho-ibuprofen-phosphate 137 or ibuprofen.

The same effect was observed for other compounds of the invention in which Z is represented by Formula Z-I.

### Example 3

### Phosphoric acid diethyl ester 4-{2-[6-fluoro-3-(4-methanesulfinyl-benzylidene)-2-methyl-3H-inden-1-yl]-acetylamino}-butyl ester (phosphosulindac amide, 106)

Phosphosulindac amide 106 was synthesized according to procedure shown in Scheme 2 below:

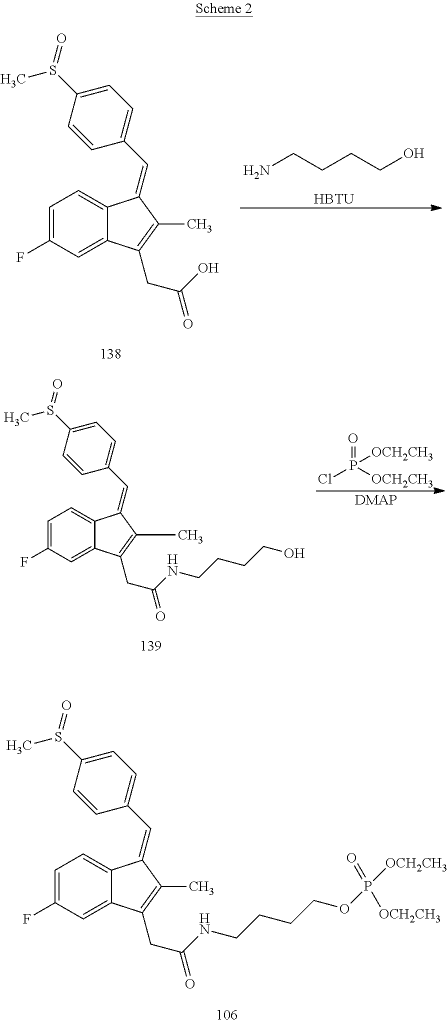

### Step 3.1 Synthesis of 2-[6-fluoro-3-(4-methanesulfinyl-benzylidene)-2-methyl-3H-inden-1-yl]-N-(4-hydroxy-butyl)-acetamide (139)

Sulindac (138) (0.356 g, 1 mmol), 4-amino-1-butanol (0.138 ml, 1.5 mmol) and HBTU (0.57 g, 1.5 mmol) were dissolved in 5 ml of DMF further containing DIPEA (0.17 ml, 1 mmol). The reaction mixture was stirred at room temperature for 4 h. The reaction was monitored by TLC. The remnant was dissolved in ethyl acetate, and then washed with 1 M HCl, saturated NaHCO₃ solution, distilled water, brine, and dried over Na₂SO₄. After the solvent was removed under reduced pressure, the crude product was purified by flash column chromatography to give 139 as a white solid in 95% yield.

### Step 3.2 Synthesis of phosphoric acid diethyl ester 4-{2-[6-fluoro-3-(4-methanesulfinyl-benzylidene)-2-methyl-3H-inden-1-yl]-acetylamino}-butyl ester (106)

Under nitrogen, diethyl chlorophosphate (0.43 g, 1.25 mmol) was added drop-wise to a solution of alcohol 139 (0.427 g, 1 mmol) in dichloromethane (10 ml) containing DIPEA (0.17 ml, 1 mmol), and DMAP (6 mg, 0.05 mmol). The reaction mixture was stirred overnight and monitored by TLC. The reaction solution was washed with water (2×25 ml), dried over anhydrous Na₂SO₄, filtered and concentrated. The crude residue was purified by column chromatography using ethanol:ethyl acetate (10:90) as eluant. The pure fractions were combined and evaporated to give a slightly yellow liquid 106 in 85% yield.

### Pharmacokinetic Studies of Phosphosulindac Amide 106 in Mice

### Methods

Mice were administered a single oral dose of 100 mg/kg of phosphosulindac amide 106 (PSA) or 66 mg/kg sulindac (equimolar to phosphosulindac amide 106) and mMice were sacrificed at designated time points when blood was collected, centrifuged immediately and the resulting plasma was deproteinized by immediately mixing it with a 2-fold volume of acetonitrile. PSA and its metabolites were analyzed by HPLC as described by Xie et al. (Xie G, Nie T, Mackenzie G, Sun Y, Huang L, Ouyang N, et al. The metabolism and pharmacokinetics of phosphosulindac (OXT-328) and the effect of difluoromethylornithine. Br. J. Pharmacol. 2011).

### Results

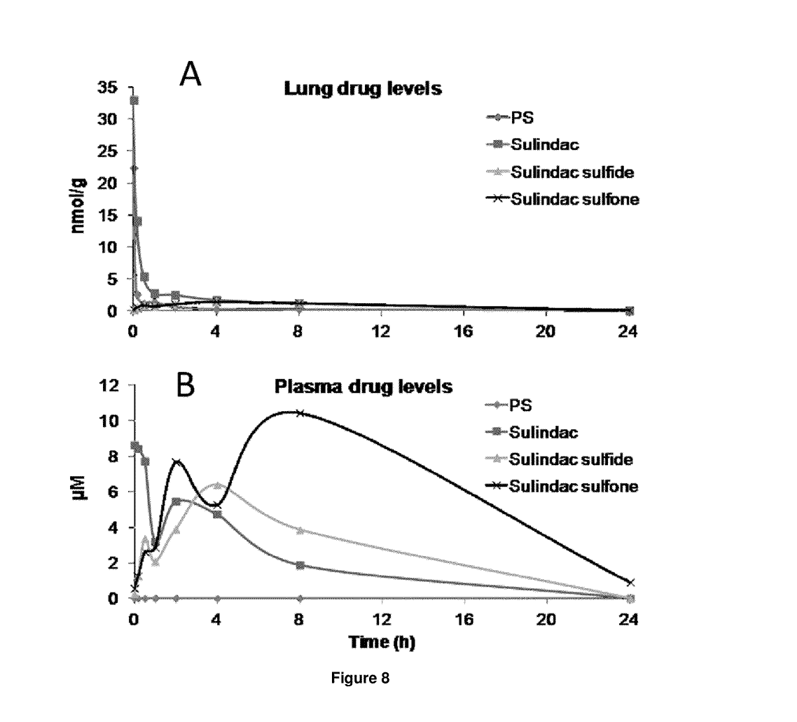

As shown in FIG. 8,
Intact PSA is detected in serum for several hours, becoming undetectable at 24 hours.
The maximum concentration of PSA was Cₘₐₓ=24 μM; the Tₘₐₓ=15 min.
Surprisingly, PSA generated no detectable sulindac or sulindac sulfone.
AUC_{0-24 h} values (μM×h)
   PSA administration
      PSA=54.04
      Sulindac sulfide=17.86
   Sulindac administration
      Sulindac=385.36
      Sulindac sulfide=690.72
      Sulindac sulfone=93.37

PS amide 106 and PI amide 105 generated significant blood and tissue levels of intact compound (FIG. 3 and FIG. 8). These levels are higher than those observed with the corresponding carboxylic ester compound.

### Compound Efficacy Inhibition of Colon Cancer

### Methods

### Efficacy in Xenografts

Female Ncr nude mice (5-6 weeks old; Harlan, Taconic Farms, Germantown, N.Y.) were inoculated subcutaneously in their right and left flanks, each with 2.0×10⁶ SW-480 colon cancer cells suspended in 100 μl of PBS. When the average tumor size reached 100 mm³, the animals were divided into two groups of 6 and treated orally for 3 weeks either with vehicle (PBS) or PSA 100 mg/kg/d in PBS. Tumors were measured twice a week with a digital microcaliper, and tumor volumes were calculated (tumor volume=[length×width×(length+width/2)×0.56]). At the end of the study, animals were sacrificed and their tumors were removed and weighed.

### Efficacy in Apc^{Min/+} Mice

Eleven week old male C57BL/6J APC^{Min/+} (n=6/group) were treated for 6 weeks with PSA 100 mg/kg/d or vehicle (corn oil) given by oral gavage. At the end of the study, animals were sacrificed and their small intestine and colon were removed, opened longitudinally and all tumors counted under a magnifying lens.

### Results

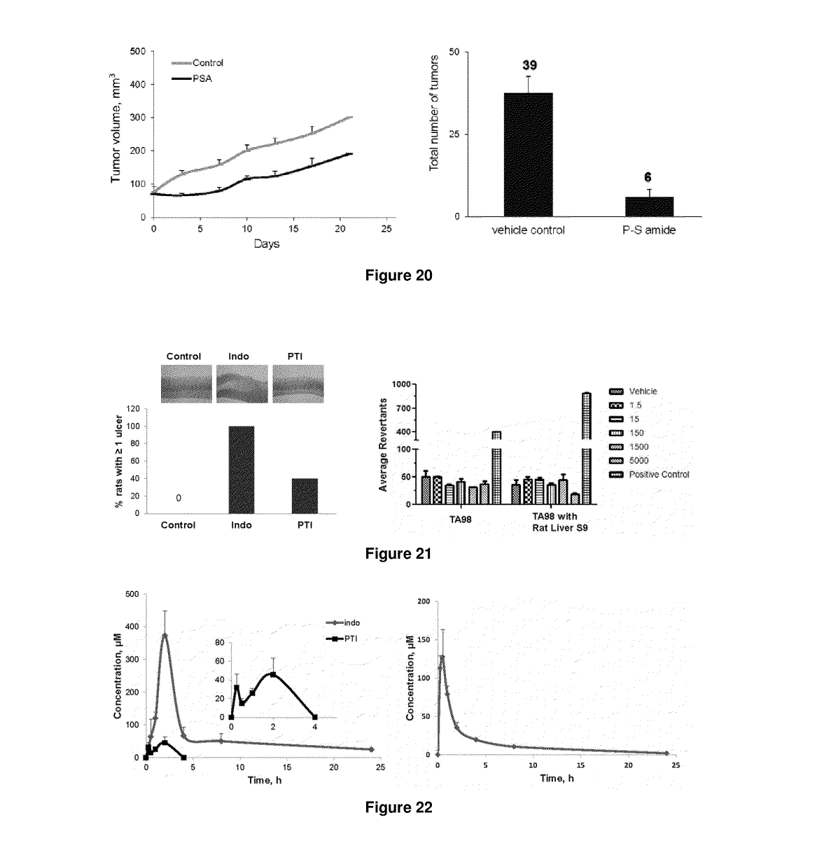

As shown in FIG. 20,
Compared to control, at the end of the study PSA 100 mg/kg/day reduced the growth of colon cancer xenografts by 41% (p<0.02)
In the Apc^{Min/+} mouse model, PSA reduced the number of all intestinal tumors by 85%, compared to the control group (p<0.001).

### Example 4

### [1-(4-Chloro-benzoyl)-5-methoxy-2-methyl-1H-indol-3-yl]-acetic acid 4-[2-(diethoxy-phosphoryloxy)-ethyl]-phenyl ester (phospho-tyrosol-indomethacin (PTI), 2)

The title compound 2 was synthesized as shown in Scheme below:

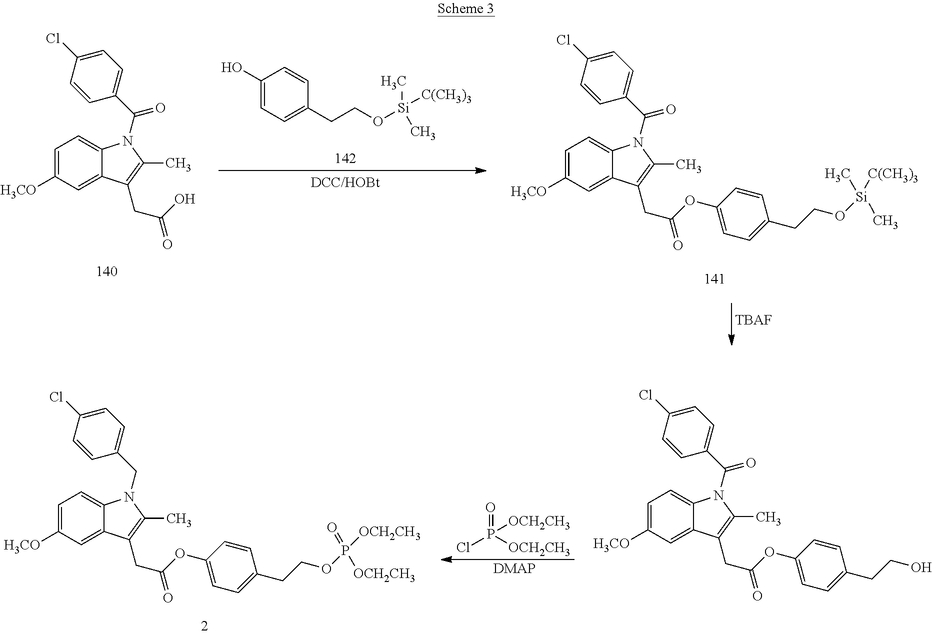

### Step 4.1 Synthesis of [1-(4-chloro-benzoyl)-5-methoxy-2-methyl-1H-indol-3-yl]-acetic acid 4-(2-hydroxy-ethyl)-phenyl ester (141)

Under nitrogen atmosphere, indomethacin (140) (1.0 g, 3 mmol), N,N′-dicyclohexylcarbodiimide (DCC) (0.9 g, 3.2 mmol), 1-hydroxybenzotriazole (HOBt) (0.6 g, 3 mmol) and dichloromethane (20 ml) were added to a flask and stirred at room temperature for 1 h. Then, a solution of the phenol 142 (0.9 g, 3.2 mmol) and DMAP (60 mg) in dichloromethane (10 ml) were added. The resulting solution was stirred at room temperature overnight. The reaction was monitored by TLC. The insoluble solids were removed by filtration and the solvent was evaporated. The remnant was dissolved in ethyl acetate, and then washed with 2% NaHCO₃ solution, distilled water, brine, and dried over Na₂SO₄. After the solvent was removed under reduced pressure, the crude product was purified by flash column chromatography to yield 141 as a pale yellow oil in 90% yield.

### Step 4.2 Synthesis of [1-(4-chloro-benzoyl)-5-methoxy-2-methyl-1H-indol-3-yl]-acetic acid 4-(2-hydroxy-ethyl)-phenyl ester (143)

Compound 141 (7 mmol) obtained in step 4.1 above was dissolved in THF (40 ml) and reacted with 1 M solution of tetrabutylammonium fluoride (TBAF) in THF (7.2 mmol) and acetic acid (7 ml) at room temperature for 3 h. Alcohol 143 was obtained as a pale yellow solid in 88% yield. MS: 477 (M+).

### Step 4.3 Synthesis of [1-(4-Chloro-benzoyl)-5-methoxy-2-methyl-1H-indol-3-yl]-acetic acid 4-[2-(diethoxy-phosphoryloxy)-ethyl]-phenyl ester

Diethylchlorophosphate (2.5 ml, 17.26 mmol) was added drop-wise to a solution of alcohol 143 (6.64 mmol) in dichloromethane (10 ml) containing DIPEA (2.2 ml, 13.28 mmol), followed by DMAP (25 mg) as a solid. The reaction mixture was heated under reflux overnight. The reaction solution was washed with water (2×25 ml), dried over anhydrous Na₂SO₄, filtered and concentrated. The crude residue was purified by column chromatography using n-hexane:ethyl acetate (40:60) as eluant. The pure fractions were combined and evaporated to give as viscous yellowish oil in 82% yield. MS: 613.16 (M+).

### Methods

Cell Culture

Human lung [A549 (p53 wild type; Kras mutant) and H358 (p53 null; Kras mutant)], colon [SW480 (p53 mutant; Kras mutant) and HT-29 (p53 mutant; Kras wild type)] and breast [MDA-MB-231 (p53 mutant; Kras mutant)] cancer cell lines (American Type Culture Collection (ATCC), Manassas, Va.) were grown in the media recommended by ATCC. F-12K medium was purchased from ATCC. McCoy's 5a medium, RPMI 1640, L-15 and antibiotics were purchased from Mediatech (Manassas, Va.). Cell viability was determined by the 3-(4,5-dimethylthiazol-2-yl)-2,5-diphenyltetrazolium bromide (MTT) assay following the protocol of the manufacturer (Roche Diagnostics, Indianapolis, Ind.).

Determination of Apoptosis by Annexin V and PI Staining

After the A549 cells were treated with the test drug in 6-well plates for 72 h, all cell populations (suspended and attached) were collected and stained with annexin V-FITC and propidium iodide (PI) (Invitrogen, Carlsbad, Calif.) for 15 min. Annexin V-FITC and PI fluorescence intensities were analyzed by flow cytometry with a FACScalibur. Annexin V(+)/PI(−) cells are early apoptotic cells; annexin V(+)/PI(+) cells are late apoptotic cells; and annexin V(−)/PI(+) cells are necrotic cells.

Determination of Cell Cycle by PI Staining.

SW480 cells were seeded in 60 mm plates and treated with the test drug for 24 h. The adherent cells were harvested and fixed with 70% ethanol for at least 30 min, washed with PBS, resuspended in 0.5 ml PBS containing RNase (50 μg/ml), and incubated for at 37° C. for 30 min. PI was then added to the solution to a final concentration of 40 μg/ml. The fluorescence intensities were analyzed by flow cytometry with a FACScalibur.

Determination of Cell Proliferation by BrdU Staining

Cells were seeded in 60 mm plates and treated with the test drugs for 16 h. Bromodeoxyuridine (BrdU; BD Biosciences, San Jose, Calif.) was added directly to the culture medium to a final concentration of 10 μM and incubated in the CO2 incubator for 30 min at 37° C., harvested, and fixed in 70% ethanol for 30 min on ice. DNA was denatured by incubating the cells with 2 N HCl/Triton X-100 for 30 min, followed by neutralization in 0.1 M Na₂B₄O₇ (pH 8.5). Ten million cells were incubated with 20 μl of anti-BrdU-FITC (BD Biosciences, San Jose, Calif.) for 30 min, cells were washed and resuspended in PBS containing 5 μg/ml propidium Iodide. Cell fluorescence intensity was analyzed by flow cytometry with a FACScalibur.

Cellular Uptake of PTI and Indomethacin

A549 cells were seeded in 100 mm² plates and allowed to grow as a monolayer. Upon reaching 80% confluence, different concentrations of indomethacin or PTI were added and the cells were incubated at 37° C. for 2 h, 6 h or 16 h. The incubation was terminated by washing the cell monolayer with complete medium and PBS. The cells were harvested by scraping, extracted by two-fold volume of acetonitrile and centrifuged at 13,000 rpm for 5 min. Drug levels were determined by HPLC.

Salmonella Plate Incorporation Mutagenicity Assay

The genetic toxicology assay was performed by BioReliance Corporation (Rockville, Md.). PTI concentrations of 5,000, 1,500, 500, 150, 50, 15, 5.0 and 1.5 μg/plate were evaluated with tester strain TA98 with and without metabolic activation in duplicate plates using the plate incorporation method of treatment. DMSO was used as the vehicle. PTI is soluble at all dose levels.

COX-1 and COX-2 Assay

The COX-1 and COX-2 inhibitory activities of indomethacin and PTI were determined with the COX fluorescent inhibitor screening assay kit (Cayman Chemical Co., Ann Arbor, Mich.) following the manufacturer's instructions.

Determination of Prostaglandin E2 (PGE2)

PGE2 levels in the cell culture media were determined by the immunoassay kit purchased from Cayman Chemical (Ann Arbor, Mich., USA) according to the manufacturer's instructions.

Efficacy in Lung and Colon Cancer Xenografts in Mice

Lung cancer treatment protocol: A549 cells (1.5×106) suspended in 100 μl of PBS (25% Hydrogel) were injected subcutaneously into both the left and right flanks of 5-6-week old female NOD SCID mice (Taconic Farms, Germantown, N.Y.). When the average tumor volume reached 100 mm3, the mice were divided into 3 groups: vehicle, PTI 10 mg/kg/d, and PTI 15 mg/kg/d (n=10/group); treatment lasted 2 weeks. At the end of the treatment, animals were euthanized and the xenografts were harvested.

Colon cancer prevention protocol: 6-week-old female athymic nude mice (Taconic Farms, Germantown, N.Y.; n=6/group) were pretreated by oral gavage with corn oil (vehicle) indomethacin 2 mg/kg/d or PTI 10 mg/kg/d for 5 days, and then 1.2×106 SW480 colon cancer cells suspended in 100 μl of PBS were inoculated subcutaneously to each flank. These two doses represent 50% of the respective maximum tolerated dose for these animals, as calculated by us. The treatment was continued for another 38 days. Tumor size was monitored by measuring the length (L) and width (W) with a digital caliper and the volume was calculated according to the formula, L×W×(L+W/2)×0.56.

Immunohistochemistry

Staining for PCNA and p-p65 (Ser276) was performed as described. Apoptosis was determined immunohistochemically by the terminal deoxynucleotidyl transferase-mediated deoxyuridine triphosphate-biotin nick end-labeling (TUNEL) assay.

Gastrointestinal Toxicity

The gastrointestinal toxicity of PTI was determined in rats following a standard protocol. Six-week-old Sprague-Dawley rats (n=5 per group) were administered by gavage for 4 days vehicle indomethacin 4.75 mg/kg/d (positive control), or PTI 10 mg/kg/d. On day 5, the animals were sacrificed and gastric toxicity was evaluated by H&E staining and light microscopy.

Statistical Analyses

Results were expressed as mean+/−S.E.M. p<0.05 was considered statistically significant. Data were analyzed using descriptive statistics and graphical displays. Tumor volumes were compared among the treatment groups using repeated-measures ANOVA. Differences were analyzed using of Pearson's modification of the x2 test.

### Results

PTI Inhibits the Growth of Human Cancer Cell Lines

We evaluated the growth inhibitory effect of PTI on human cancer cell lines originating from colon (SW480, HT29), lung (A549, H358) and breast (MDA-MB-231). Their IC50 was measured after 72 h of indomethacin or PTI treatment. As shown in FIG. 26A, the range of 72 h-IC50 for PTI was from 23 μM (MDA-MB-231) to 87 μM (HT29), suggesting that breast cancer cell lines were more sensitive to PTI, whereas colon cancer cell lines (HT29) were the most resistant. Compared to indomethacin, PTI was more potent in all five cell lines, with the potency enhancement ranging from 6 to 30 fold.

Cell Kinetic Effect of PTI on Human Cancer Cell Lines

The cytokinetic effect of PTI was measured in order to assess its mechanism of cell growth inhibition. Cell proliferation was evaluated by bromodeoxyuridine incorporation. As shown in FIG. 26B, PTI reduced Brd-U incorporation in A549 cells in a concentration-dependent manner. At 60 μM PTI decreased the proportion of BrdU positive cells by 96%. In contrast, equimolar amounts of indomethacin only reduced BrdU positive cells by 15%.

Cell cycle analysis showed that treatment of cells with 1×IC50 PTI induced a significant G1-to-S block, with the proportion of cells in G0/G1 phase increasing from 56.8 to 69.7%. The percentage of cells in G0/G1 phase is much higher than that following treatment with indomethacin at equimolar concentration (G0/G1: 57.7%). Of note, this effect became prominent only at 72 h, with a trend towards significant changes in cell cycle phase distribution being present at 24 and 48 h. Thus, our findings suggest that PTI blocks G1-to-S transition more potently than indomethacin. Annexin V-PI staining showed that PTI induced concentration-dependent apoptosis in A549 cancer cells in vitro. Both early and late apoptosis were present, but the former predominated. At 72 h, in A549 cells, the annexin V+ cells increased from 7.5% in control to 12.3% at PTI 1.5×IC50 and to 71.4% at 2×IC50 (FIG. 26C). These results indicate that the in vitro cytokinetic effect of PTI encompasses cell proliferation, cell cycle and apoptosis.

Cellular Uptake

In order to compare the cellular penetration of PTI and indomethacin, we assessed their uptake by A549 cells. Cellular uptake of PTI was dose- and time-dependent. After 6 h of incubation, the cellular uptake of PTI (50 to 200 μM) was about 200-fold greater than that of indomethacin. After 16 h, the cellular uptake of PTI was about 230-fold greater than that of indomethacin. These findings suggest that PTI has a markedly greater ability to penetrate cancer cells compared to its parental compound.

Effect of Esterases on the Stability of PTI in Vitro

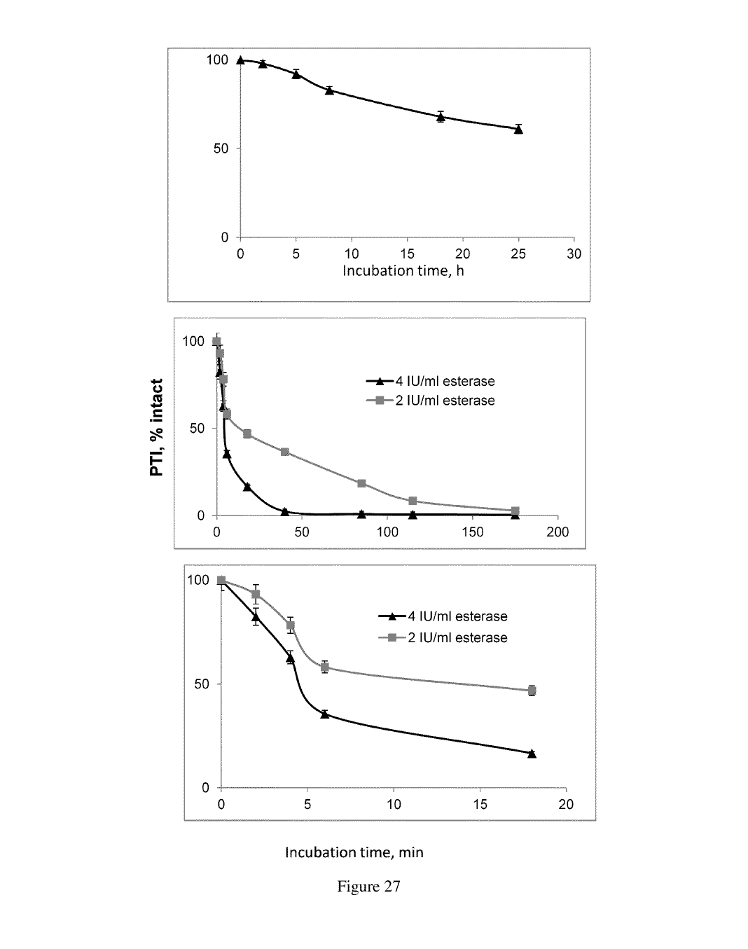

Stability of PTI is critical for its pharmacological activity. Hydrolysis of the intact drug by esterases leads to significant attenuation of its cytotoxicity in vitro. This hydrolysis markedly depends on esterase concentration. In complete media containing 10% serum, PTI is slowly hydrolyzed starting at 1 h of incubation, with 40% hydrolyzed after 24 h (FIG. 27). To determine its half-life, PTI was incubated in vitro with purified porcine liver esterases at 2 IU/ml and 4 IU/ml. As shown in FIG. 27, the breakdown of PTI in the presence of 4 IU/ml esterase (half-life: 2 min) was more rapid compared to the 2 IU/ml esterase (half-life: 5 min).

In the presence of 4 IU/ml of porcine liver esterase, we observed approximately 20% intact PTI remaining after 20 min of incubation. Although the stability and integrity of PTI were affected by esterase, PTI is to some degree stable in the presence of pure liver esterase.

Pharmacokinetics

We evaluated the pharmacokinetics of PTI and indomethacin in mice. As shown in FIG. 22 (upper panel), after a single intraperitoneal injection of PTI its plasma levels reached the maximum concentration (Cmax=46 μM) at 2 h and became undetectable at 4 h. Indomethacin is the major metabolite of PTI, reaching a maximum concentration of 378 μM at 2.5 h, and could be detected in blood 24 h post administration. Compared to PTI, a single intraperitoneal administration of an equimolar dose of indomethacin resulted in a peak plasma level of 127 μM at 1 h, and became negligible 24 h post administration (FIG. 22, lower panel). The AUC0-24 h of PTI plus its metabolite was 1700 μM×h, while that of indomethacin was 500 μM×h. Our results show that the bioavailability of PTI is significantly higher (3.5 fold) compared to that of indomethacin.

PTI Shows Less Gastrointestinal Toxicity, and no Cardiotoxicity or Genotoxicity in Rats

We evaluated the safety of PTI by examining its gastrointestinal toxicity, cardiotoxicity and genotoxicity and compared this with conventional indomethacin.

Gastrointestinal Toxicity

Rats were administered vehicle, indomethacin (4.75 mg/kg/day) and PTI (10 mg/kg/day) by gavage for 4 days (FIG. 21). At sacrifice on day 5, 100% of the rats treated with indomethacin developed ulcers compared to 40% of the PTI-treated rats, as shown in FIG. 4A, representing a 60% reduction in gastrointestinal toxicity (p<0.01).

Cardiotoxicity

Heart tissue sections from mice treated with PTI for 1.5 months were examined and scored histologically, following H&E staining and light microscopy for tissue damage and for the presence of inflammatory cells. No differences in cardiotoxicity were observed between PTItreated and control mice.

Genotoxicity

The genotoxicity of PTI was evaluated by measuring its ability to induce reverse mutations of two bacterial strains of Salmonella typhimurium (TA98 and TA100) in the presence and absence of metabolic activation (rat liver S9). In the tested concentration range (1.5 to 5000 μg/plate), with or without rat liver S9, PTI showed a frequency of revertants close to that of the vehicle, but far less than that of the positive control (FIG. 21). These studies indicate that PTI has no significant genotoxicity.

In Vivo Efficacy

The ability of PTI to inhibit the growth of A549 and SW480 human cancer cell xenografts was investigated. We conducted two studies: a lung cancer treatment study and a colon cancer prevention study. In the lung cancer treatment study, A549 human non-small cell cancer cells were injected subcutaneously to SCID mice. When the tumors reached 100 mm3, mice were treated with PTI at 10 or 15 mg/kg/d for 2 weeks. As shown in FIG. 20, PTI suppressed tumor growth and the effect became statistically significant (p<0.05) starting 11 days after the initiation of treatment. The anti-tumor effect of PTI was dose-dependent. At the end of the study, PTI 10 and 15 mg/kg/d reduced tumor volume by 68% and 91%, respectively, compared to the control group.

In the prevention study, SW480 colon cancer cells were inoculated subcutaneously into nude mice following pretreatment for 5 days with vehicle, PTI or indomethacin, which were each given by oral gavage. As shown in FIG. 20, compared to control, PTI 10 mg/kg/d reduced tumor growth by 69% at the end of the study, while indomethacin had no significant effect compared to control. The effect became statistically significant starting on day 20 of treatment.

PTI inhibited the growth of cancer xenografts via a potent cytokinetic effect. Sections from A549 xenografts were stained for PCNA expression (proliferation marker) or by TUNEL (apoptosis marker). Cell proliferation in PTI-treated tumors (40.2±4.5%) was reduced by one-third compared to controls (59.7±7.5%). Interestingly, apoptosis index was almost doubled from 3.3±0.3% in control to 5.8±1.2% in PTI treated mice, representing an increase of 75.8% (FIG. 20).

Signaling Effects of PTI

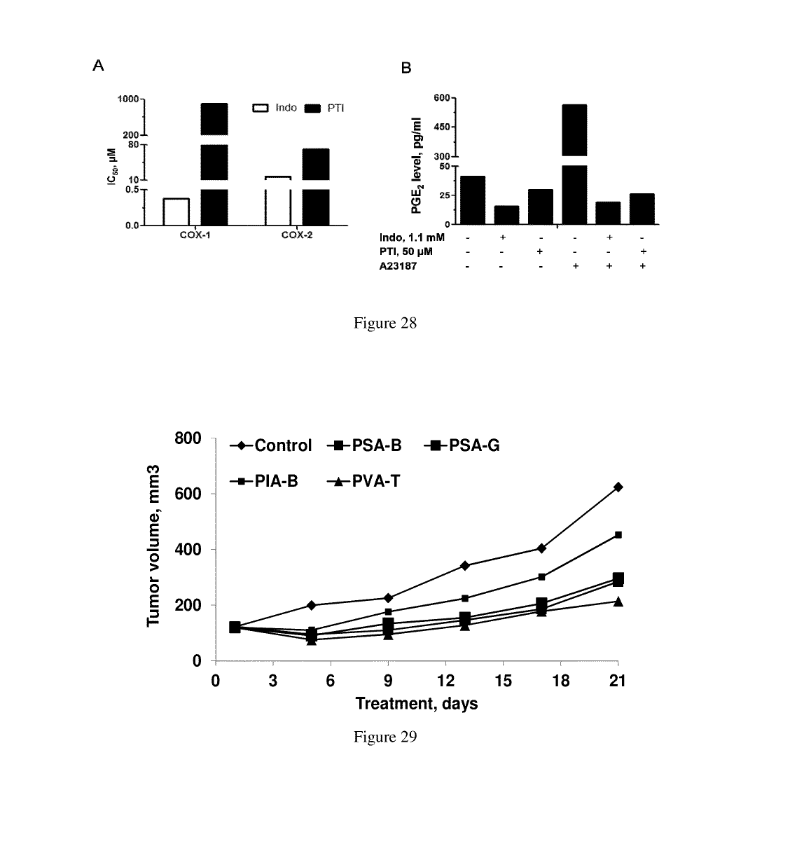

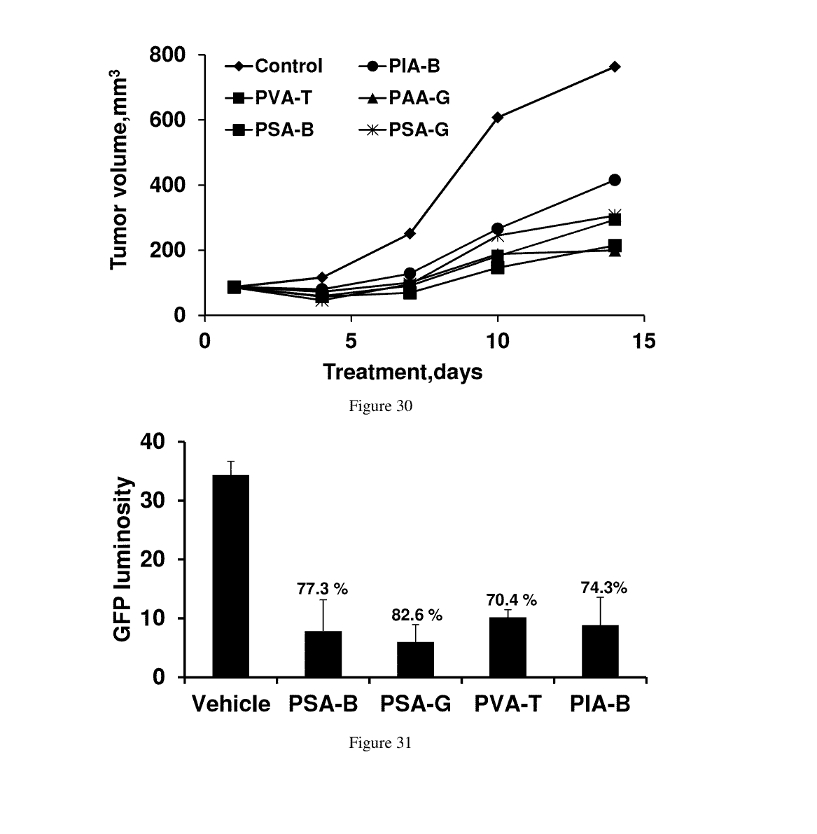

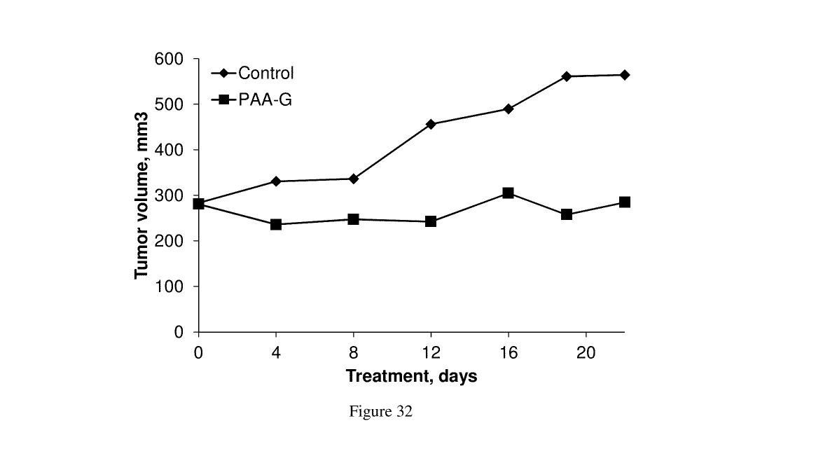

By analogy to indomethacin, a strong COX inhibitor, we investigated the effects of PTI on the COX pathway, examining COX activity, PGE2 production and NF-κB activation. COX assays with purified COX-1 and COX-2 showed that PTI inhibits COX-2 more potently than COX-1 (FIG. 28), with a 12.6-fold selectivity for COX-2 (IC50: 71.7 μM) over COX-1 (IC50: 905 μM). Indomethacin was a strong inhibitor for both COX-1 (IC50: 0.38 μM) and COX-2 (IC50: 18.2 μM) under the same assay conditions. FIG. 28 shows the effect of PTI and indomethacin on PGE2 production by A549 cells. Indomethacin at 1.1 mM (2×IC50) was more potent than PTI at 50 μM (2×IC50) in reducing the basal PGE2 production in A549 cells. Additionally, both PTI and indomethacin prevented the increase in PGE2 levels induced by the calcium ionophore A23187. The inhibitory activity of PTI and indomethacin may be a result of COX-2 inhibition.

To further explore the mechanism of action of PTI, we investigated the activation of NF-κB in the A549 lung tumor xenografts from the control and PTI-treated groups, by determining Ser276 phosphorylation of the p65 subunit of NF-κB. A shown in FIG. 28, compared to controls, PTI decreased the levels of p-p65 Ser276 in tumor xenografts by 90.0% (p=0.004). These results establish that PTI down-regulates COX and NF-κB signaling in lung cancer cells.

### Example 5

### PEGylated Phospho-Ibuprofen

A modified methodoly of H-phosphonate synthesis by Trirosh et al. (Tirosh O, Kohen R, Katzhendler J, Gorodetsky R, Barenholz Y. Novel synthetic phospholipid protects lipid bilayers against oxidation damage: Role of hydration layer and bound water. J. Chem. Soc. Perkin Trans. 2. 1997:383-9) was employed for the preparation of PEGylated phospho-ibuprofen (PI-PEG) 110.

Accordingly, the title compound was synthesized as shown in Scheme 4 below.

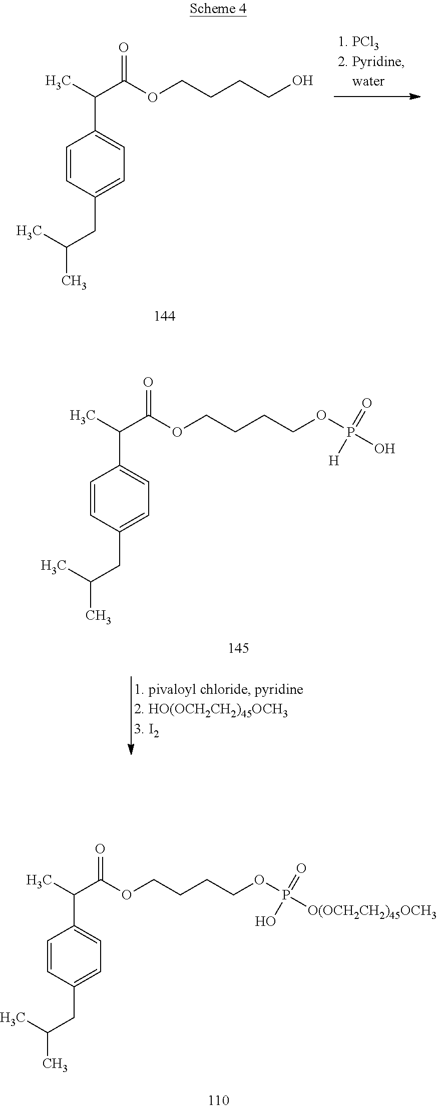

### Step 5.1 Synthesis of the H-Phosphonate 145

A stirred solution of phosphorus trichloride in dichloromethane was prepared and a solution of ibuprofen-butanol 144 was added in equimolar amounts. The stirring was continued for 30 min until the mixture was quenched by the addition of 100 ml of water-pyridine (1:4 v/v). After 15 min, the compound was extracted with chloroform from the reaction mixture, washed twice with water and dried using Na₂SO₄. The organic solvent was removed by rotary evaporation.

### Step 5.2 Synthesis of PEGylated Phospho-Ibuprofen

The residue obtained in step 5.1 above was dissolved in 50 ml of dichloromethane. Lyophilized mPEG, pivaloyl chloride and pyridine were added to the reaction and the solution was stirred for 10 min followed by removal of the organic solvent by rotary evaporation. A solution of I₂ in water-pyridine (1:1 v/v) was added to oxidize the H-phosphonate 145. The oxidation was stopped by adding 100 ml of 5% aqueous sodium thiosulfate solution. The final product, PI-PEG 110, was extracted from the aqueous medium with chloroform, which was then washed with water and brine, dried over magnesium sulfate and finally evaporated under reduced pressure. The solid residue was purified by acetone precipitation.

The isolated PI-PEG was characterized by ¹H-NMR and its purity was confirmed by both HPLC and ¹H-NMR.

### Animal Studies

Mice were treated with various amounts of PI-PEG by oral, i.p. and i.v. administration. The maximum dosage used for i.v. treatment was 1600 mg/kg and for i.p. and oral treatment 4000 mg/kg. In all cases, PI-PEG was dissolved in phosphate buffered saline pH 7.4 (PBS). No signs of toxicity, discomfort or changes in the normal mouse behavior were observed.

### Pharmacokinetics in Mice

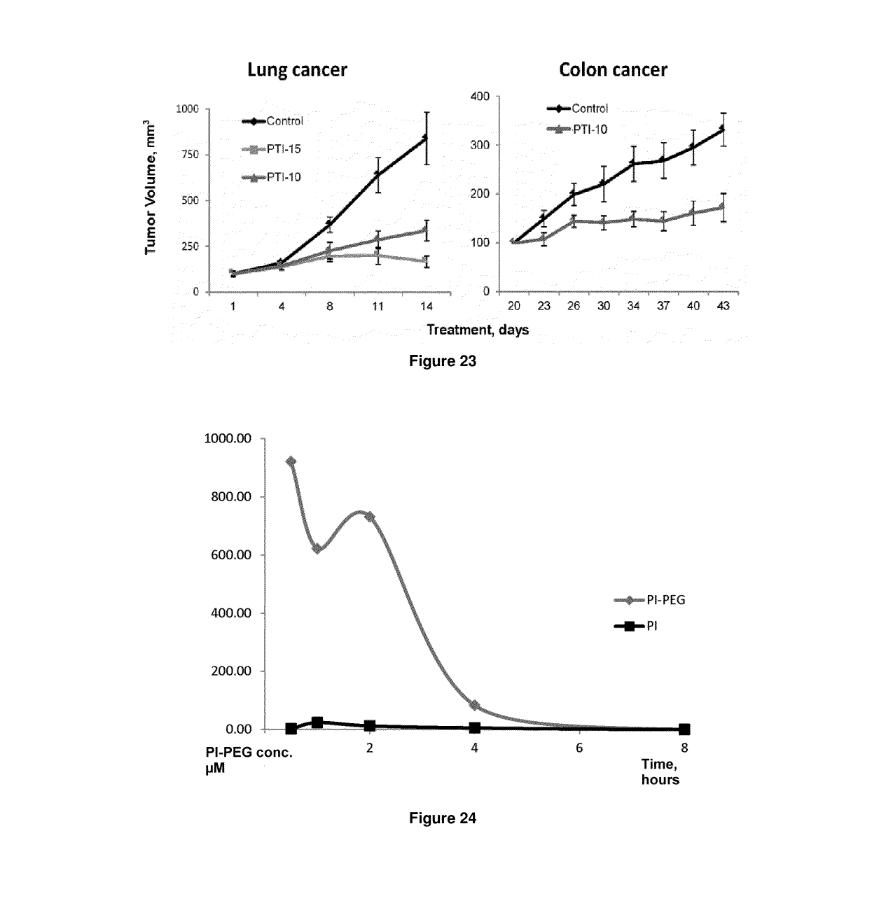

PI-PEG and phospho-ibuprofen PI 132 were injected i.p. in mice at equimolar doses and at predetermined time points the animals were sacrificed and blood was collected through heart puncture. PI-PEG and PI 132 were extracted by adding a 2-fold volume of acetonitrile. After centrifugation for 10 min at 5000×g, the supernatants were subjected to HPLC analysis. PI-PEG exhibited prolonged stability and improved circulation times compared to PI 132 as shown in FIG. 24, while PI 105 was rapidly hydrolyzed to its metabolite, ibuprofen, whose levels are not shown in FIG. 24. This demonstrates the superiority of the pegylated compounds over the corresponding non-pegylated carboxylic aced esters.

### Anticancer Efficacy Studies

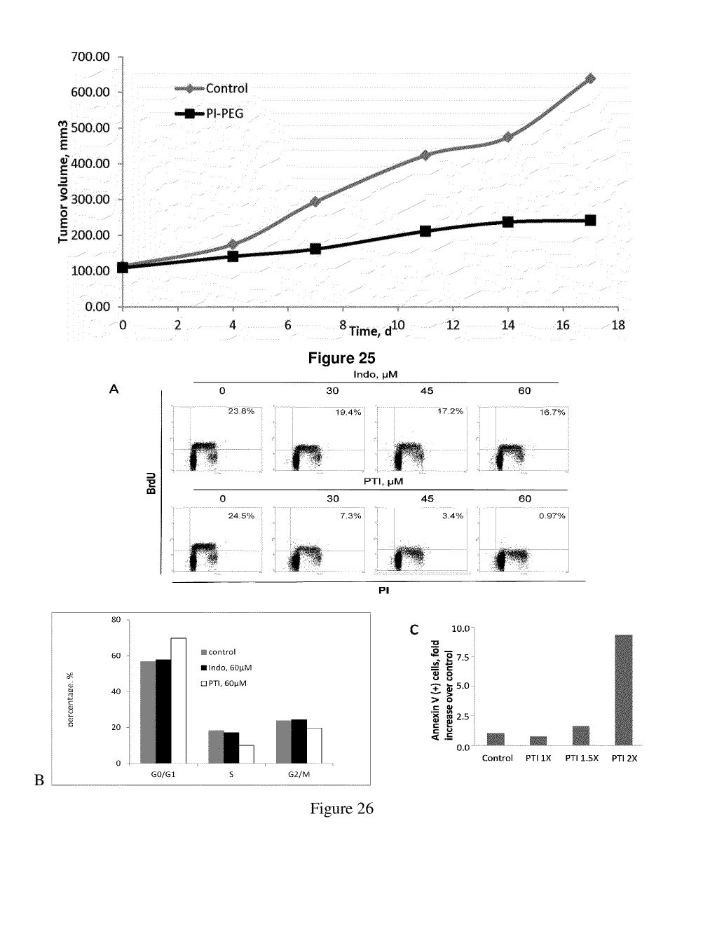

A tumor growth mouse model was used to assess the potential anticancer efficacy of PI-PEG. Human colon cancer SW-480 xenografts in nude mice were treated with daily ip injection of PI-PEG 4,000 mg/kg in PBS. FIG. 25 shows a 72% tumor growth inhibition after 18 days of treatment compared to controls (p<0.01).

Additionally, we used Apc^{Min} mice, a mouse model of colon cancer (Lipkin M, Yang K, Edelmann W, Xue L, Fan K, Risio M, et al. Preclinical mouse models for cancer chemoprevention studies. Annals of the New York Academy of Sciences. 1999; 889:14-9), to determine the efficacy of PI-PEG 110 in tumor prevention. We administered to Apc^{Min} mice 2400 mg/kg of PI-PEG orally once a day, 5 times per week for 10 weeks. At the end of the 10^{th} week, PI-PEG reduced the number of tumors on the gastrointestinal track of these mice by 80% compared to the control group (n=8 mice/group). This effect was even pronounced in the tumors of the colon (93% reduction); note that Apc^{Min} mice grow tumors in both the small intestine (predominantly) and the colon. Of interest, PEG alone administered at an equimolar dose to a third group of Apc^{Min} mice (n=8) had no effect on their number of tumors.

### Example 6

### Analgesic Effect of Compounds of the Invention

We determined in mice the analgesic effect of phosphosulindac (carboxylic ester) 118, PI (carboxylic ester) 132, PI-PEG 110 and PI amide 105 by measuring their antinociceptive effect to an acute thermal stimulus. We employed the hot-plate test following a standard protocol by Bannon A W (Bannon A W. Models of Pain: Hot-plate and formalin test in rodents. Current Protocols in Pharmacology: John Wiley & Sons, 1998).

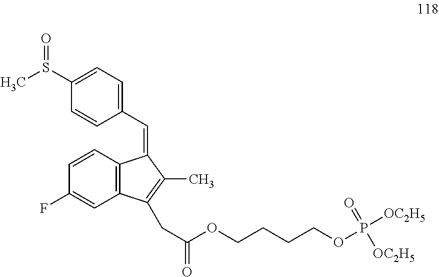 [SMILES: CCOP(=O)(OCC)OCCCCOC(=O)CC1=C(C)/C(=C/c2ccc(S(C)=O)cc2)c2ccc(F)cc21]

### Methods

Tested compounds: PS 118, PI 132, PI amide 105, each at 100 mg/kg and PI-PEG 110 1,600 mg/kg
Animals: Male CD mice (Charles River Labs), 25-30 g, divided into 5 study groups (n=5-8).
Testing: After 30 min of acclimation to the test room environment, baseline measurements were performed, mice were administered a single intraperitoneal dose of each test compound or vehicle (control). Thirty min post dosing, each animal was placed on a 55° C. hot plate and we recorded the latency to respond, i.e. the time until the animal shows a nociceptive response.

### Results

At 30 min we obtained the following latency values (seconds; mean±SD)

| | | |
|---|---|---|
| Control | 1.91 ± 0.46 | |
| PI amide 105 | 3.80 ± 0.46 | p < 0.05 |
| PI 132 | 8.53 ± 0.81 | p < 0.001 |
| PI-PEG 110 | 5.21 ± 0.74 | p < 0.01 |
| PS 118 | 4.55 ± 1.00 | p < 0.01 |

Note:
p values refer to the comparison to control.

It can be concluded that all compounds tested had a significant analgesic effect.

### Example 7

### Aerosol Administration of Phospho-Sulindac 118 (PS) Prevents Non-Small Cell Lung Cancer

Inhalation exposure system: Air flow in the system was controlled by two major devices by using the arrangement illustrated by FIG. 1: (1) an inlet air regulator which pushes air into the system via the baffle; and (2) a vacuum pump which draws air from the system.

PS was dissolved in ethanol. PS solution in the baffle was aerosolized with the ultrasonic atomizer. The aerosol passed through an ascending stainless steel column, followed by a reflux column which is maintained at a temperature gradient by a heating tape (82° C.) and a chiller (5° C.) to condense and remove ethanol. PS aerosol exiting the reflux column then passed through a charcoal column which served to remove residual traces of ethanol from aerosol before it entered the animal-holding chamber. Experimental animals were held in nose-only air-tight tubes for designated time intervals.

Orthotopic lung cancer model: BALB/c nude mice (7 weeks old) were divided into control and treatment groups (15 mice/group) and treated following a prevention protocol by administration of either aerosol generated from ethanol (control) or PS solution (treatment) for one week. The optimized exposure time and dose to mice were 50 mg/mL PS for 8 min, respectively. On day 1 of week 2, a small incision (˜5 mm) was made to the left side of the chest of anesthetized mice and 1 million GFP-A549 human lung cancer cells (A549 cells expressing green fluorescence protein (GFP) which allows their detection and quantification) were injected into their left lung as described by Y. Doki et al. (Br. J. Cancer, 79, 7-8, pages 1121-1126, 1999). Inhalation treatment was resumed 2 days post-surgery and continued for 6 weeks when mice were euthanized, and blood and lung tissues were collected. Luminosity of the GFP-A549 tumors was measured and the lungs were weighed.

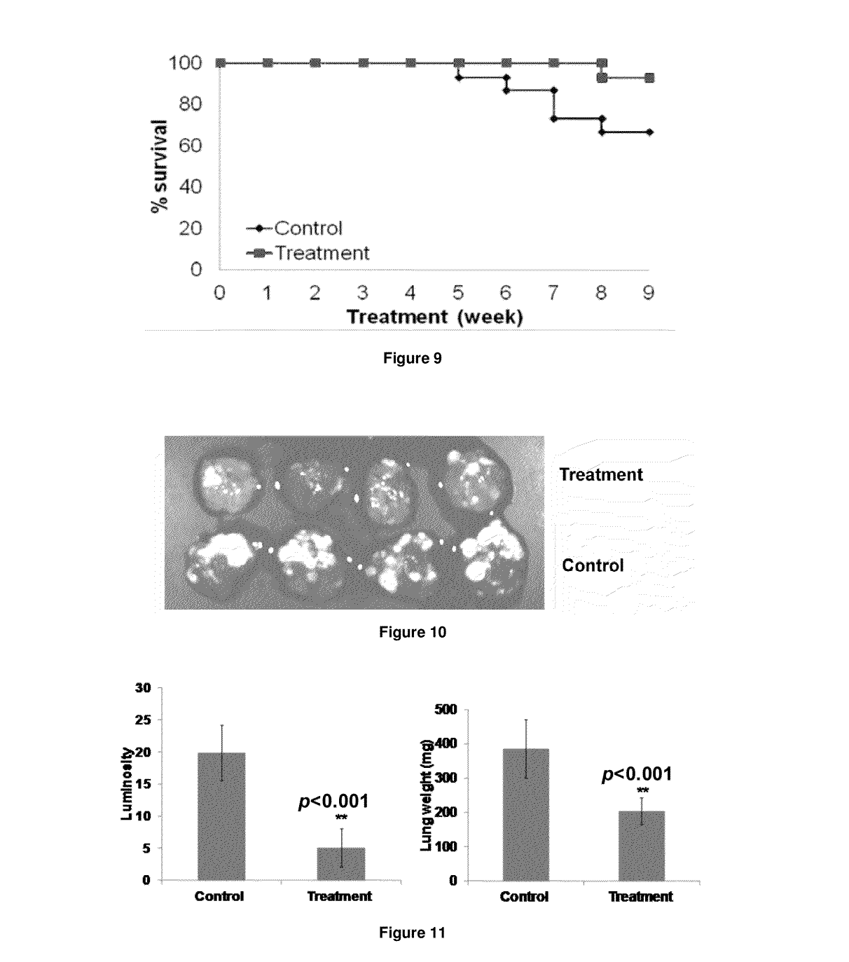

Chemopreventive efficacy: Two outcomes were used to gauge efficacy, animal survival and tumor size.
a) Survival: At the end of the study, 40% of the mice in the control group died from the disease while the death rate in the treatment group was less than 10% (p<0.03). The results are illustrated by FIG. 9.
b) Tumor size: At sacrifice, the tumor size was (all values, Mean±SEM) determined a) by luminosity: control=19.85±4.33, treatment=5.05±2.97 (p<0.001). The results are shown in FIG. 10 (upper photograph: after treatment; lower photograph: control group) and FIG. 101 (left hand side); and b) by lung weight: control=385.7±85.2 mg, treatment=204.4±39.4 mg (p<0.001). The results are shown in FIG. 11 (right hand side).

### Example 8

### The Pharmacokinetic Parameters of PS After Inhalational Administration

PS as well as sulindac, sulindac sulfide 146 and sulindac sulfone 147, the structures of which are shown below, were administered to BALB/c nude mice.

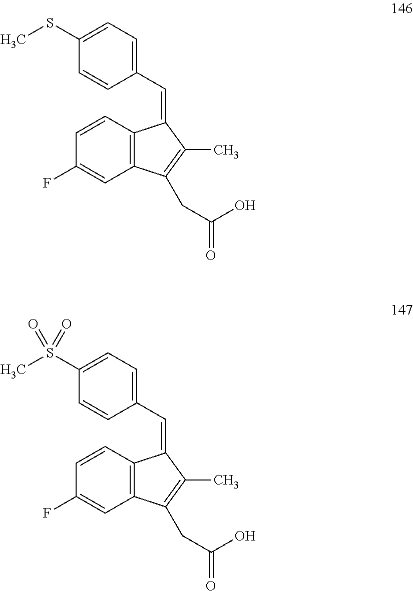 [SMILES: CSc1ccc(/C=C2/C(C)=C(CC(=O)O)c3cc(F)ccc32)cc1 ; CC1=C(CC(=O)O)c2cc(F)ccc2/C1=C\c1ccc(S(C)(=O)=O)cc1]

After 8 min of inhalation treatment, BALB/c nude mice were euthanized at various time points and drug levels were analyzed by HPLC in plasma and lung tissues. The results are summarized below and are further illustrated in FIG. 8.

TABLE 1

Pharmacokinetic parameters in lung

| | AUC | Cₘₐₓ, nmol/g | Tₘₐₓ, h |
|---|---|---|---|
| PS 118 | 7.7 | 22.2 | 0 |
| Sulindac | 30.1 | 32.9 | 0 |
| Sulindac sulfide 146 | 18.9 | 1.4 | 4 |
| Sulindac sulfone 147 | 57.5 | 4.6 | 8 |

TABLE 2

| | Pharmacokinetic parameters in plasma | | |
|---|---|---|---|
| | AUC | Cₘₐₓ, μM | Tₘₐₓ, h |
| PS 118 | 0 | 0 | — |
| Sulindac | 49.5 | 8.6 | 0 |
| Sulindac sulfide 146 | 66.9 | 6.4 | 4 |
| Sulindac sulfone 147 | 142.4 | 10.4 | 8 |

These findings indicate the following: a) inhalation provides intact PS to the lungs, which is more cytotoxic to human cancer cells than either of its three metabolites, sulindac, sulindac sulfide 146 and sulindac sulfone 147; b) oral administration does not provide intact PS to the lungs, leading only to its three metabolites; and c) there are sufficient concentrations of sulindac and its metabolites in the circulation, and for prolonged periods of time. Sulindac, sulindac sulfide 146 and sulindac sulfone 147 are established cancer chemopreventive agents and thus, when derived from inhaled PS, they can prevent smoking/nicotine-related cancers at sites other than the lung.

### Example 9

### Inhalation Delivery of Aerosolized Phospho-Sulindac to the Lungs of Mice Leads to Higher Drug Levels than Oral Administration

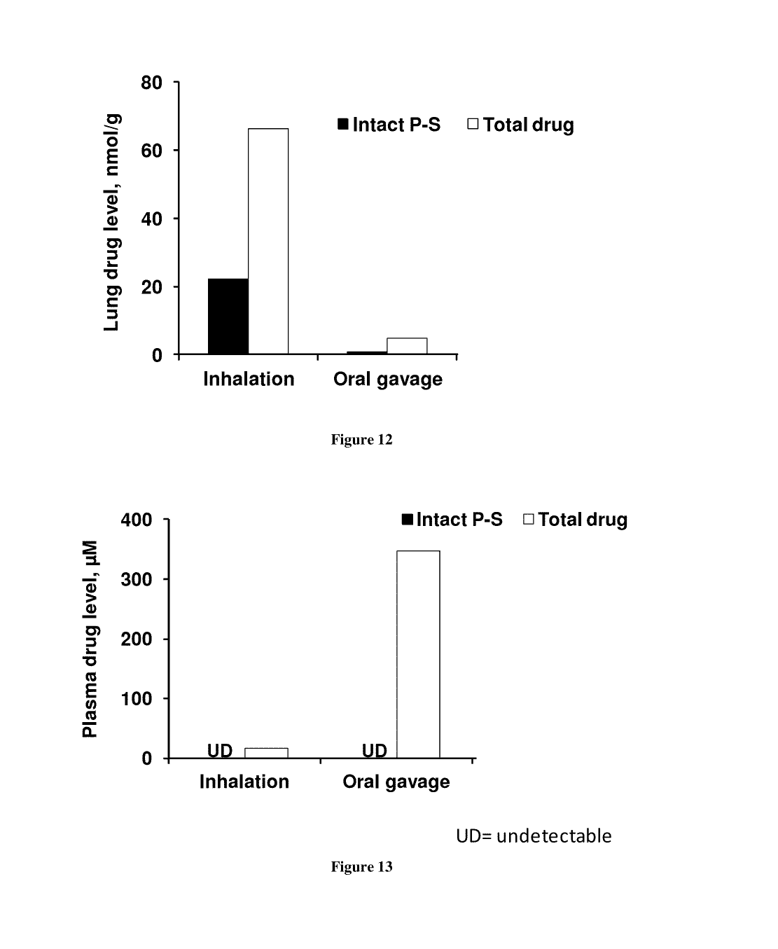

The delivery of aerosolized phospho-sulindac 118 (PS) to the lungs of mice was evaluated using the same inhalation device as in Example 8 and compared to its oral delivery. The PS doses were: inhalational=6.5 mg/kg body weight; oral=150 mg/kg body weight. The level of PS in the lungs and plasma after inhalation vs. after oral gavage are shown in FIGS. 12 and 13, respectively.

Lungs: PS levels: The aerosol-exposure system delivered a high level of intact PS to the lungs of mice (>20 nmol/g); while there were only trace levels of intact PS (<2 nmol/g) by oral administration.

Total drug levels: It represents the total level of PS plus its metabolites. The main metabolites of PS are sulindac, sulindac sulfide 137 and sulindac sulfone 138; at least the first two can cause gastrointestinal and renal side effects. The levels achieved by inhalation were significantly higher compared to those by oral administration.

Plasma: PS levels: undetectable.

Total drug levels after inhalation treatment (17 μM) was lower than that after oral (348 μM) administration. Thus, inhalation delivery leads to blood levels of sulindac that can be chemopreventive for various non-lung cancers, but which are not particularly high so that can have significant potential toxicity. Of the three main metabolites of PS, at least sulindac and sulindac sulfide 137 can cause gastrointestinal and renal side effects.

Thus, PS can be effectively delivered to lung cells by inhalation of a mixture of tobacco smoke with aerosolized PS.

### Example 10

### Inhibition of Glioblastoma Cell Lines

U87 cells were treated with sulindac and ibuprofen as well as with the compounds. U87 is a human primary glioblastoma cell line, formally known as U-87 MG. This cell line has epithelial morphology, and is one of the most frequently used glioblastoma cell lines. In this experiment the 24-hour growth inhibitory concentration (24-h IC₅₀) of phospho-sulindac, phospho-ibuprofen, phospho-ibuprofen glycerol, and phospho-ibuprofen glycerol amide were determined, as specified by Huang et al. (Huang L, Mackenzie G G, Sun Y, Ouyang N, Xie G, Vrankova K, et al. Cancer Res. 2011; 71: pp. 7617-27).

The values of 24-h IC₅₀, μM are summarized in Table 3 below.

TABLE 3

| | 24-h IC₅₀, μM |
|---|---|
| Sulindac | ≧1000 |
| Ibuprofen | ≧1000 |
| Phospho-sulindac 118 | 114 |
| Phospho-ibuprofen 132 | 98 |
| Phospho-ibuprofen glycerol 93 | 105 |
| Phospho-ibuprofen glycerol amide 94 | 87 |

Thus, the compounds of the present invention inhibited glioblastoma cell lines U87 with enhanced potency compared to conventional NSAIDs sulindac and ibuprofen.

### Example 11

### Phosphovalproic Acid 134 (PV) and Ibuprofen Phospho-Glycerol Amide (PGIA) Synergize Strongly to Inhibit the Growth of Glioblastoma and Lung Cancer

### Methods

Cell growth: After treatment with PV or PGIA alone or in combination for 24 h, the reduction of 3-(4,5-dimethylthiazol-2-yl)-2,5-diphenyltetrazolium bromide dye (MTT), was determined following the manufacturer's protocol (Promega, Madison, Wis.).

Apoptosis: Cells (1.0×10⁵ cells/well) were treated with or without PV, PGIA or valproic acid (VPA) for 24 or 48 h. After treatment, cells were trypsinized, stained with Annexin V-FITC (100× dilution, Invitrogen) and propidium iodide (PI) 0.5 μg/ml and the fluorescence intensity was analyzed by FACScaliber.

### Results

PGIA is a Successful Combination Partner with PV in Inhibiting Glioblastoma Cell Growth in Vitro

The potential synergy of PV and PGIA was screened in vitro. Isobologram established synergy between PV and PGIA.

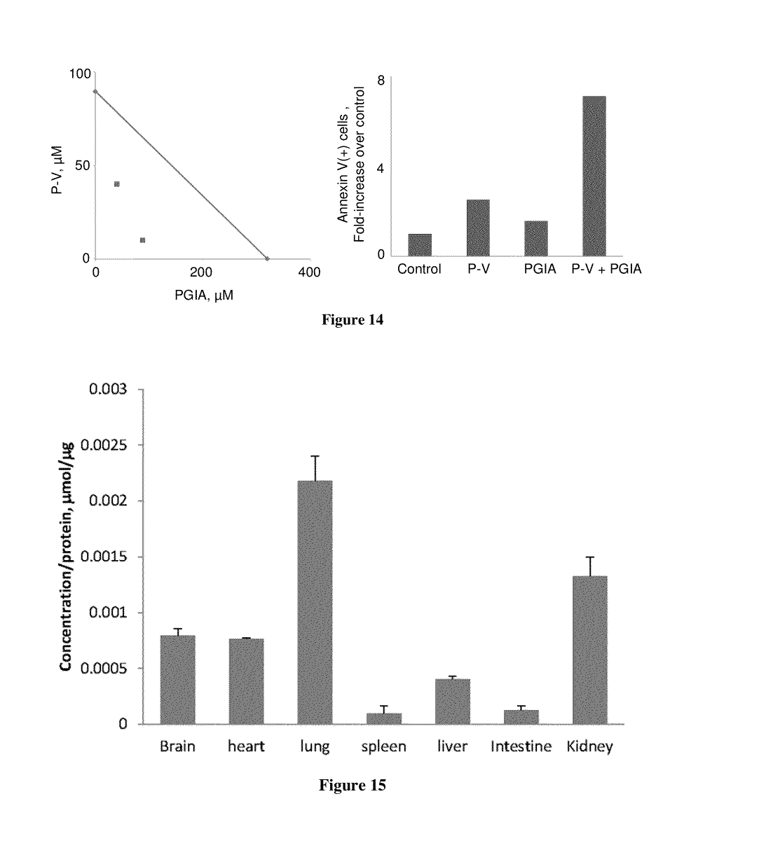

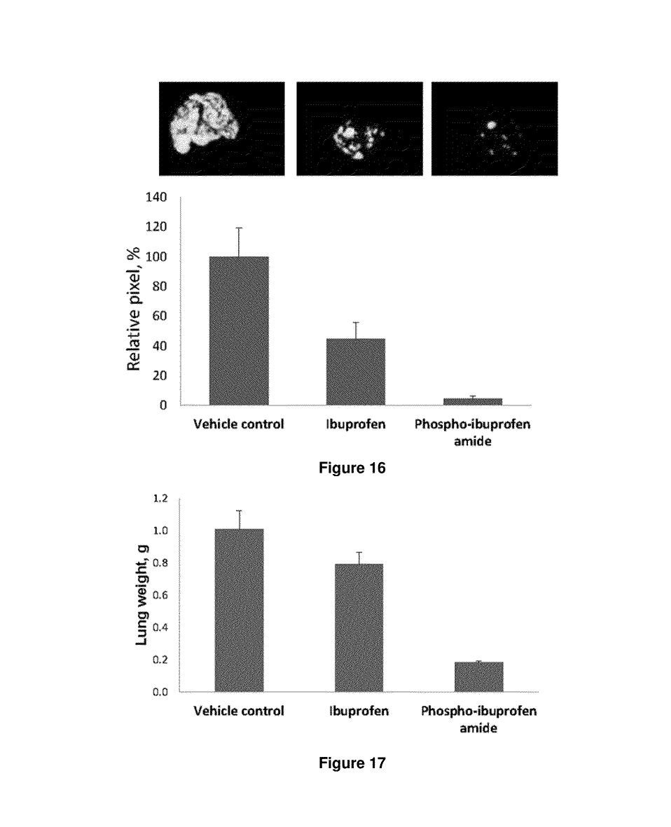

It was observed that in cultured U87 glioblastoma cells, there is a clear-cut pharmacological synergy between PV and PGIA (FIG. 14, left panel). In addition, there is also a synergistic effect in the induction of apoptosis. For example, after 24 h of incubation with PGIA 200 μM and PV 40 μM, the fold-increase of annexinV (+) cells was 8.0, compared to 3.0 for PV 40 μM alone and 1.8 for PGIA 200 μM (FIG. 14, right panel).

Similar results were obtained in other glioblastoma cell lines, such as U118, LN-18 and LN-229, as well as in A549 lung cancer and MIA PaCa-2 pancreatic cancer cell lines.

### Example 12

### In Vivo Efficacy of Several Compounds of the Invention Against Gastric, Skin, and Lung Cancer Models

### Test Compounds:

Phospho-sulindac amide (butane spacer) (BSA-B) 106
Phospho-sulindac amide (glycerol spacer) (PSA-G) 102
Phospho-ibuprofen amide (butane spacer) (PIA-B) 105
Phospho-valproic acid amide (tyrosol spacer) (PVA-T) 139
Phospho-aspirin amide (glycerol spacer) (PAA-G) 108

### Gastric Cancer—Subcutaneous Model

Drugs: all suspended in corn oil and given by i.p. once/day, 6 times/week for 3 weeks PSA-B=200 mg/kkg
PSA-G=200 mg/kg
PIA-B=160 mg/kg
PVA-T=100 mg/kg
Study summary: The AGS human gastric cancer cells (4 million cells in 100 μL PBS-Matrigel, 1:1, v/v) were implanted subcutaneously into both flanks of athymic nude mice. Treatment with each drug (once/day, 7 days/week) was started when the average tumor volume reached ˜150 mm³. Tumor volumes were recorded twice a week.
Results: All compounds inhibited tumor growth as follows: PSA-B=67.7%, PSA-G=64.9%, PIA-B=33.8%, and PVA-T=81.2%. All results were statistically significant (p=0.0009 to 0.05) except for PIA-B (trend).

### Skin Cancer—Intradermal Model

Drugs: all formulated in hydrogel and applied topically 2 times/day, 6 days/week for 2 weeks PS hydrogel contains 2% of PS by weight. George told me that all the other hydrogels have similar drug content.

The dose of drugs (PSA-B, PSA-G, PIA-B, PVA-T and PAA-G) was estimated to be 80 mg/kg.

Test compounds were formulated in a hydrogel—and applied topically.

Study summary: The A431 human squamous cell carcinoma cells (1 million cells in 100 μL complete DMEM medium) were implanted intradermally into both flanks of SCID Beige mice. Treatment with the hydrogel-formulated drugs (once/day, 6 days/week) was started when the average tumor volume reached ˜150 mm³. Tumor volumes were recorded twice a week.
Results: All compounds inhibited tumor growth as follows: PSA-B=81.1%, PSA-G=67.3%, PIA-B=51.5%, PVA-T=69.4%, and PAA-G=83.5%. All effects were statistically significant (p=0.008 to 0.01).

### Lung Cancer—Orthotopic Model

Drugs: all suspended in corn oil and given by i.p. once/day, 6 times/week for 6 weeks
PSA-B=200 mg/kkg
PSA-G=200 mg/kg
PIA-B=160 mg/kg
PVA-T=100 mg/kg

Study summary: GFP-A549 cells (4 millions in 200 μL PBS) were injected into the tail vein of athymic nude mice. These cells express stably the green fluorescence protein (GFP) that allows their detection. Each test compound was administered i.p. dissolved in corn oil. At the end of the study, mice were euthanized and the lungs were collected for documentation of GFP luminosity.
Results: All compounds inhibited tumor growth as shown. All effects were statistically significant (p=0.001 to 0.0001).

### Example 13

### PAA-G Inhibits the Growth of Human Breast Cancer (MCF-7) Xenografts in Nude Mice. We Assessed the Chemotherapeutic Potential of PAA-G in MCF-7 Xenografts

PAA-G (150 mg/kg), suspended in corn oil, was given by i.p. once/day, 6 times/week for 3 weeks.
Xenografts: MCF-7 cells (1.5×10⁶) were implanted subcutaneously into both flanks of nude mice.
Treatment: The treatment was started when the average tumor size reached ˜280 mm³, mice were treated with vehicle and PAA-G (150 mg/kg, i.p. in corn oil).
Results: PAA-G potently suppressed tumor growth, causing tumor regression from day 4 to day 12, and maintaining tumor stasis throughout the study period. At the end point, the tumor volume of the vehicle was 564±110 mm³ and that of PAA-G was 285±39 mm³, representing a 98% tumor growth inhibition (p<0.005) compared to control.
Plasma and tumor drug levels: significant amounts of intact PAA-G were detected in both the blood (85 μM) and tumors (684 pmol/mg protein) in mice given PAA-G via i.p. route.

### Example 14

### Methods of Drug Incorporation into Hydrogels

### Pluronic-Based Method:

A mixture of Pluronic P123 and PS 118, dissolved in tetrahydrofuran (1:10, w/w), was dialyzed for 24 h at room temperature through a membrane (molecular weight cutoff of 3,500 Da) in phosphate-buffered saline, which was replaced three times. The dialysis bag was then wiped with absorbent paper and through dialysis with high molecular weight PEG (PEG: 900,000) the water was removed to concentrate the solution inside the bag until gel formation. The final drug loading onto the gel was 1.4% (w/w), while the polymer constituted 27-30% w/v of the gel. Similar hydrogel formulations were created also for the rest of our drugs. Drug loading was not significantly altered from the different drugs incorporated.

Utilizing the above method, we produced also hydrogels with PS that incorporated in the initial mixture 2.5, 5 and 10% (w/w) of oleic acid. Oleic acid is a permeation enhancer; as shown later oleic acid actually enhanced the permeation of PS into mice and into human skin.

Note: For experimental controls, we prepared control gel (=gel without drug) by the so-called cold method, where 28% w/v of Pluronic P123 was dispersed slowly in PBS at 2-5° C.

### Propylene Glycol-Based Method

A PS hydrogel formulation with propylene glycol (PG) was also prepared. Briefly, a solution of PS dissolved in PG is mixed with equal volume of water. Pluronic P123 is added creating a final ratio of drug:PG:P123 0.7:15:12 by weight and the mixture is heated at 100° C. until a homogenized solution is created. The mixture is allowed to cool down to room temperature.

### Example 15

### Efficacy Against Skin Papillomas

### Skin Papillomas:

Successful formulation of PS and DFMO in hydrogel: Hydrogels are prepared from cross-linked polymers that provide sustained local delivery of therapeutic agents. We formulated PS (PSG) and DFMO (DFMOG) in a hydrogel as already described, using Pluronic 123, a biocompatible triblock copolymer based on polyethylene glycol. PSG has the following properties: drug loading=3.3±0.5% w/w (mean±SEM for this and all subsequent values); polymer content=28±0.7% w/v; diameter of micelles derived from the gel when diluted in water=35±10 nm; polydispersity index=0.309±0.13; and stability over 6 weeks at room temp: a) physical properties: no appreciable changes; and b) chemical stability=97.8±2.2%. DFMOG has similar properties (data not shown); of note, the DFMO content of DFMOG was 5% but it can exceed 10%.

Topically applied PS plus DFMO is efficacious in the treatment of papillomas: Papillomas were induced in FVB mice by topical application of dimethyl-benz[a]anthracene (DMBA; 100 nmol, single application) and tetradecanoyl-phorbol-acetate (TPA; 6.8 nmol, 2×/wk). This initiation-promotion protocol leads to papillomas by wk 10, and in a fraction of the mice to SCC by wk 20. On wk 10 the mice had multiple papillomas (˜4/mouse), and were randomized into 8 groups (12 mice/group) for topical or oral treatment with PSG, DFMOG, or their combination for which PSG and DFMOG were mixed 1:1 (v/v). Criteria similar to those used for human papilloma classification were applied to assess disease progression: hyperplasia→papilloma (Pap 1-3)→microinvasive SCC (M1-3)→invasive SCC. On wk 20, the study was terminated.

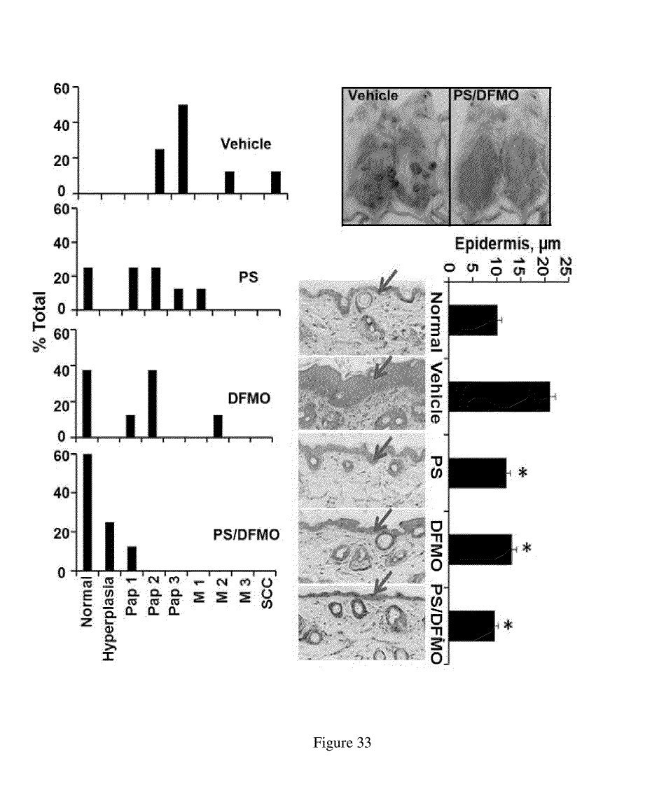

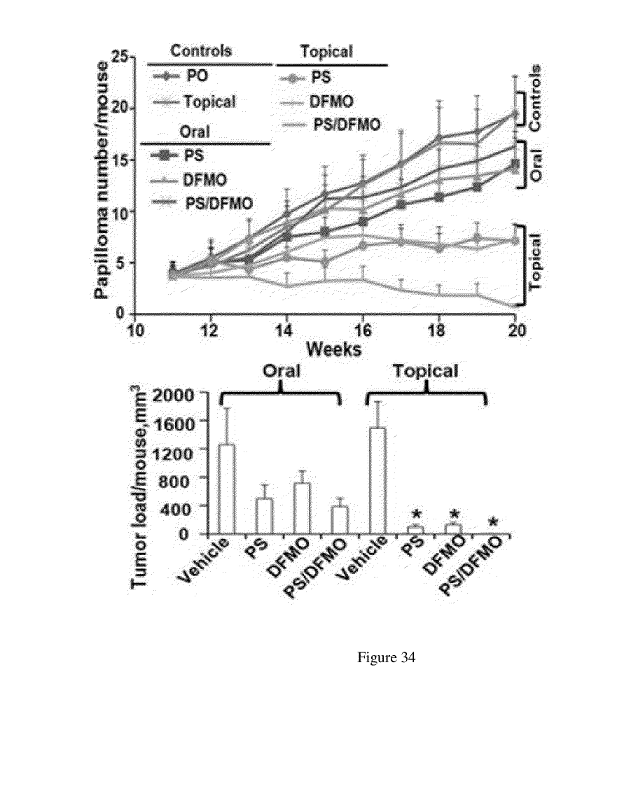

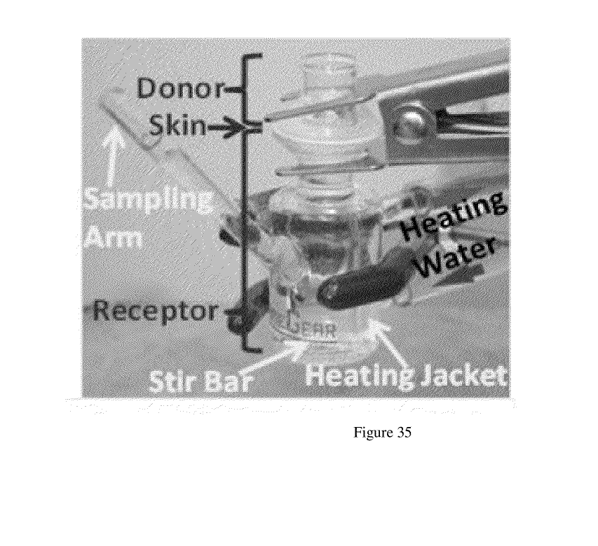

### Results (FIGS. 33 and 34):

Topical application: PS/DFMO dramatically suppressed papillomas:
Tumor number (papillomas/mouse). PS/DFMO reduced it by 97% (control=19.70±3.50 vs. PS/DFMO=0.7±0.2; p<0.001). Moreover, 58% of the mice were disease-free (7/12). PS and DFMO, each given alone, merely stabilized disease progression and failed to regress it.
Tumor burden (average tumor volume/mouse): PS/DFMO reduced it by 99.9% (control=1496.1±368.9 vs. PS/DFMO=0.7±0.3; p<0.001). PS and DFMO alone reduced it by ˜90% (p<0.01).
Histopathological analyses showed that 58% (7/12) of mice treated with PS/DFMO were normal; 25% (3/12) had hyperplasia; 17% (2/12) had grade 1 papillomas; and none had lesions grade 2 or above. All control mice developed at least grade 2 to 3 papillomas, and 25% had micro-invasion or SCC. PS and DFMO alone were less efficacious. In addition, the thickness of the epidermis was increased in animals exposed to DMBA+TPA compared to normal (unexposed) mice. PS/DFMO returned epidermis thickness back to normal, while PS and DFMO alone had a partial effect.

Oral administration of PS, DFMO, and PS/DFMO failed to reduce papilloma multiplicity or change tumor histopathology, and the reduction in tumor load was not significant (p>0.05).

### Topical Application Delivers Drugs to the Papillomas and Minimizes Their Systemic Distribution.

We determined by HPLC the levels of PS and its metabolites in papillomas treated with PSG or oral PS; samples were obtained at sacrifice, 1 hr post last dose. Results: The level of PS in papillomas treated with PSG was 256.0±36.4 nmol/g, and the combined levels of its three main metabolites, sulindac, sulindac sulfide and sulindac sulfone, was 9.1±0.4 (<4% of PS). Of note, even with a small number of samples, there was a clear trend indicating an association between PS levels and papilloma size. In papillomas treated with an equal dose of PS orally, the level of PS was 0.3±0.2 nmol/g and that of its main metabolites was 1.9±0.2 nmol/g. Thus, topical administration of PS delivered 135-fold more PS to the target site than oral. This finding is consistent with the reported extensive first-pass metabolism of oral PS. No papillomas were available from the PS/DFMO group.

A PK study of the topical application of PSG to mice showed that it minimizes systemic exposure to PS and its metabolites (AUC_{0-24 h}=65 μM*h), being 31-fold lower compared to oral administration (AUC_{0-24 h}=2,012 μM*h), thereby reducing the risk of adverse side effects. Other PK parameters for the topical administration of PS: Blood: Cₘₐₓ=0, as expected. Skin: AUC_{0-24 h}=683.8 μM*h, Cmax=36.1 μM, Tₘₐₓ=2 h.

Careful inspection and histological examination of the skin where PS/DFMO was applied showed no local reaction to them. The stomach, duodenum, small intestine and the heart (coronary arteries) had no macroscopic or microscopic (H&E) evidence of toxicity. We also assayed the blood levels of indicators of renal (creatinine and urea), pancreatic (amylase) and liver (transaminases, bilirubin, alkaline phosphatase) function and hemoglobin/hematocrit (anemia and, indirectly, bleeding). No differences from normal levels were noted (commercial lab; data not shown). These findings agree with those from other preclinical animal models in which PS was consistently safe, in contrast to sulindac.

It was determined by applying PSG or DFMOG to human skin samples (AlloSource, Centennial, Colo.) using the Franz Cell chamber (described later). We consistently obtained PS levels of ˜2 μmoles/g tissue and of DFMO ˜5 μmoles/g tissue.

Oleic acid (OA) (10%) was added to PSG (PSG-OA). PSG and PSG-OA were applied for 1 h to the skin of live mice or to human skin in a Franz Cell chamber and PS levels were determined by HPLC.
Results: a) OA 10% stimulated the delivery of PS. PS, nmole/g tissue: Mice: PSG=39.8±1.6; PSG-OA=150.0±16.7, p<0.01; 3.8-fold increase. Human skin: PSG=1572±231; PSG-OA=3306±321, p<0.01; 2.1-fold increase. And b) OA 10% also stimulated the delivery of DFMO both in mice (2.3-fold) and in human skin (2.1-fold) (data not shown).

We explored the effects of PS and DFMO on cytokinetics and cell signaling. PS/DFMO suppressed cell proliferation, polyamine levels and EGFR expression in vivo; and suppressed β-catenin signaling in vitro. Specifically, in DMBA/TPA-induced papillomas, PS/DFMO

a) suppressed cell proliferation by 50%: The proliferation index was: control=67.4±5.3 vs. PS/DFMO=33.7±2.9; p<0.01);

b) suppressed polyamine levels: putrescine by 25%: control=77±9.2 vs. PS/DFMO=58±2.1; spermidine by 45%: control=305±24.0 vs. PS/DFMO=168±12.3; spermine by 30%: control=40±6.7 vs. PS/DFMO=28±3.0 (p<0.05 for all) Similar results were obtained in cultured skin cells; and

c) suppressed EGFR expression: DMBA-TPA increased the percentage of phospho-EGFR (+) skin cells by >2-fold over normal skin, and PS/DFMO returned it to the level of normal skin.

To study β-catenin signaling, we expressed P-catenin (TOPflash or the mutant FOPflash) in HEK293T cells and studied the effect of PS and/or DFMO on its transcriptional activity.
Results (luciferase/renilla activity): Control=1.23±0.11; PS 5 μM=0.76±0.03; DFMO, 5 μM=1.18±0.01; PS 5 μM plus DFMO 5 μM=0.44±0.01 (p<0.01-0.04). Thus PS and DFMO displayed pharmacological synergy in their inhibition of P-catenin signaling.

### Other Embodiments

All publications, patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What is claimed is:

1. A method for treating non-cancerous conditions of the skin or mucous membranes, said method comprising topically administering to a subject in need thereof an effective amount of a compound of Formula V: 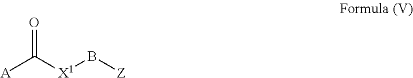
wherein A is an optionally substituted aliphatic, heteroaliphatic, aromatic, heteroaromatic substituent or alkylaryl substituent having 1 to 100 carbon atoms;
X¹ is selected from —O—, —S—, and —NR⁵—;
R⁵ is selected from hydrogen and a C₁₋₆ alkyl;
B is an aliphatic, alicyclic, heteroaliphatic, heterocyclic, aryl, aralkyl, or heteroaromatic group optionally substituted with one or more R¹⁵ moieties,
each R¹⁴ is independently, selected from hydrogen, halogen, hydroxyl, alkoxyl, —CN; an optionally substituted aliphatic, alicyclic, heteroaliphatic, heterocyclic, aryl, aralkyl, heteroaromatic moiety; —OR^{R}, —S(═O)ₙR^{d}, —NR^{b}R^{c}, —C(═O)R^{a} and —C(═O)OR^{a}; n is 0-2; R^{a}, for each occurrence, is independently selected from hydrogen and an optionally substituted aliphatic, alicyclic, heteroaliphatic, heterocyclic, aryl, aralkyl, or a heteroaromatic moiety; each of R^{b} and R^{c}, for each occurrence, is independently selected from hydrogen; hydroxyl, SO₂R^{d}, and aliphatic, alicyclic, heteroaliphatic, heterocyclic, aryl, aralkyl, heteroaromatic or an acyl moiety; R^{d}, for each occurrence, is independently selected from hydrogen, —N(R^{e})₂, aliphatic, aryl and heteroaryl, R^{e}, for each occurrence, is independently hydrogen or aliphatic; and R^{R} is an optionally substituted aliphatic, alicyclic, heteroaliphatic, heterocyclic, aryl, aralkyl, heteroaromatic or acyl moiety;
Z is selected from: 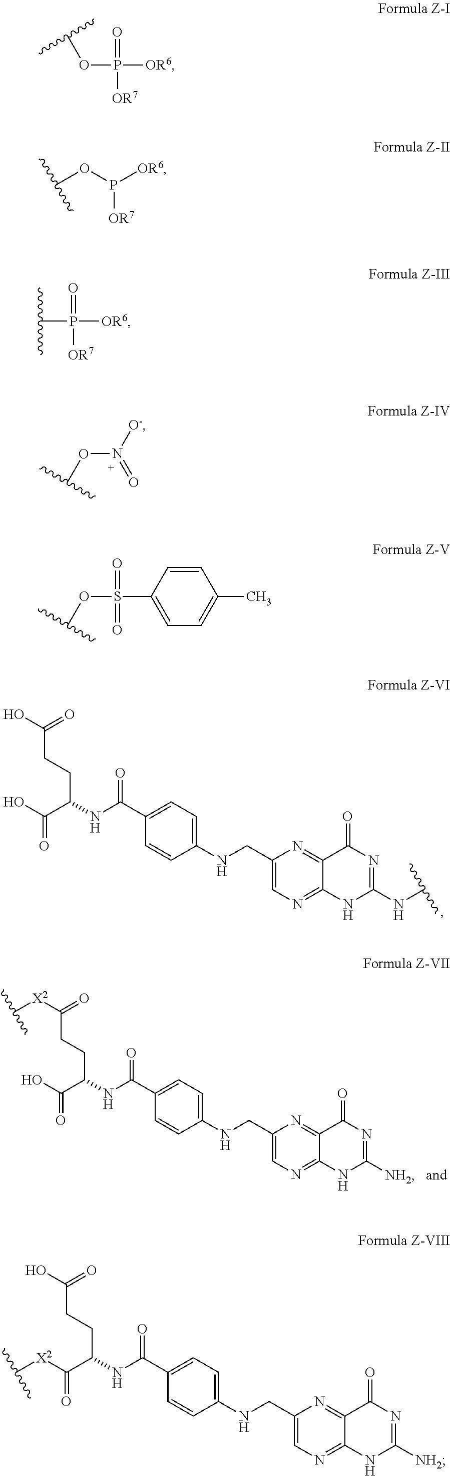
or B together with Z forms a structure: 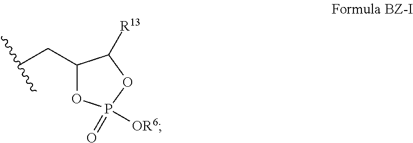
R⁶ and R⁷ are independently selected from hydrogen, C₁₋₆-alkyl, and polyethylene glycol residue; and
R¹³ is selected from hydrogen, an aliphatic group with 1 to 22 carbon atoms (e.g. C₁₋₆-alkyl), and polyethylene glycol residue;
or a pharmaceutically acceptable salt thereof.

2. The method of claim 1, wherein said compound is a compound of Formula (I): 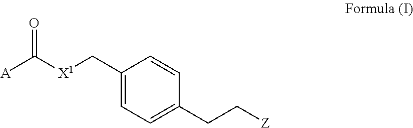
or a pharmaceutically acceptable salt thereof, wherein
A is an optionally substituted aliphatic, heteroaliphatic, aromatic, heteroaromatic substituent or alkylaryl substituent having 1 to 100 carbon atoms or selected from: 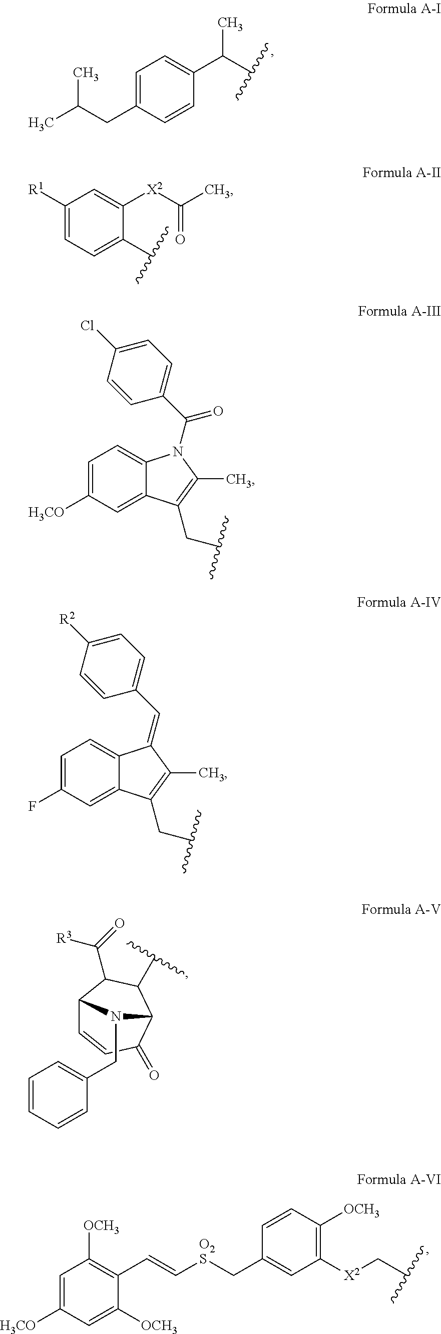 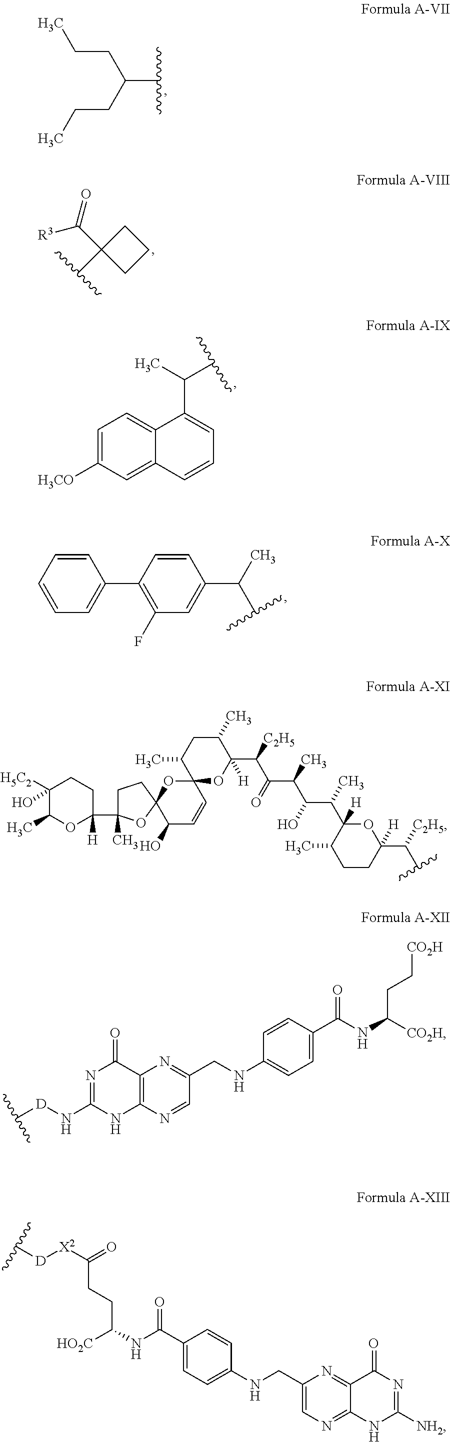 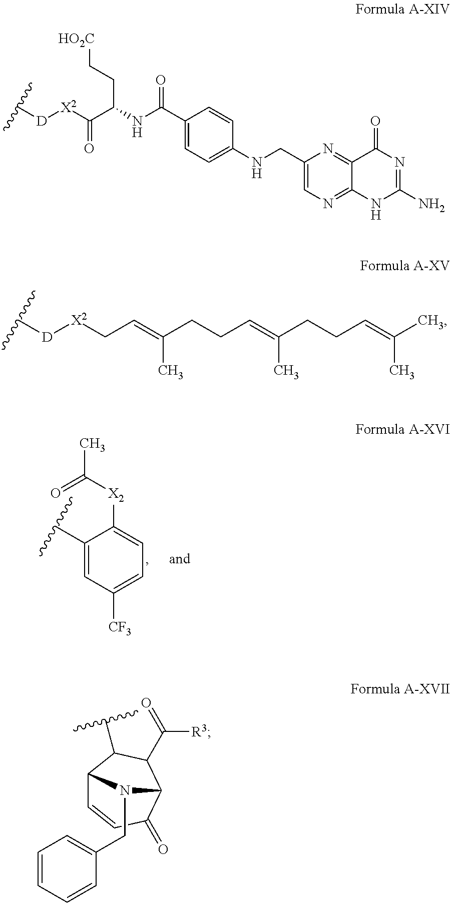
D is absent or 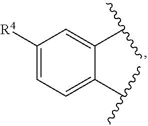
X¹ and X² are independently selected from —O—, —NR⁵—, and —S—;
R¹ and R⁴ are independently selected from hydrogen and trifluoromethyl;
R² is selected from —SCH₃, —S(O)CH₃, and —S(O)₂CH₃;
R³ is selected from hydroxyl, Z, —X¹—(CH₂)₄-Z, and 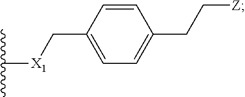
R⁵ is selected from hydrogen and C₁₋₆ alkyl;
Z is selected from: 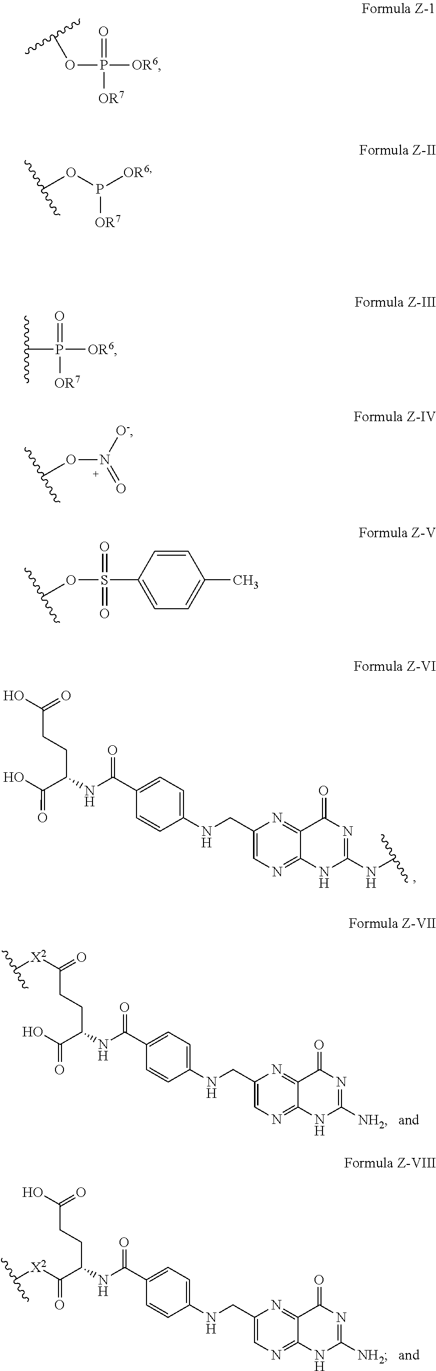
R⁶ and R⁷ are independently selected from hydrogen, C₁₋₆-alkyl, and polyethylene glycol residue.

3. The method of claim 2, wherein said compound is selected from compounds 1 to 21 and 109 or a pharmaceutically acceptable salt thereof.

4. The method of claim 1, wherein said compound is a compound of Formula (II): 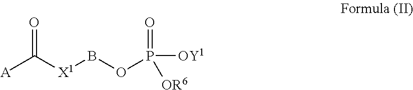
or a pharmaceutically acceptable salt thereof, wherein
Y¹ is a polyethylene glycol residue;
R⁶ is selected from hydrogen, C₁₋₆-alkyl, and polyethylene glycol residue;
A is an optionally substituted aliphatic, heteroaliphatic, aromatic, heteroaromatic substituent or alkylaryl substituent having 1 to 100 carbon atoms or selected from: 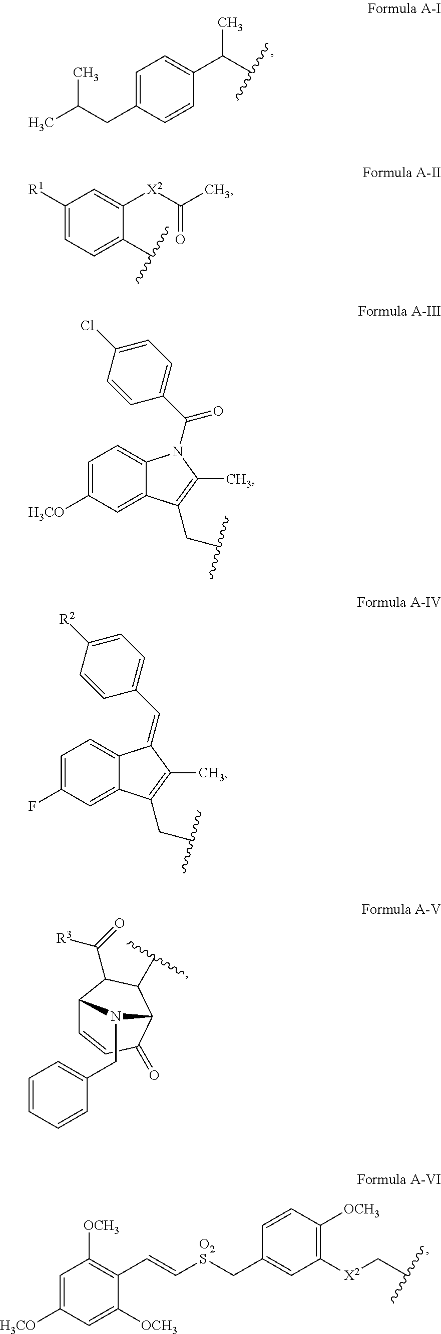 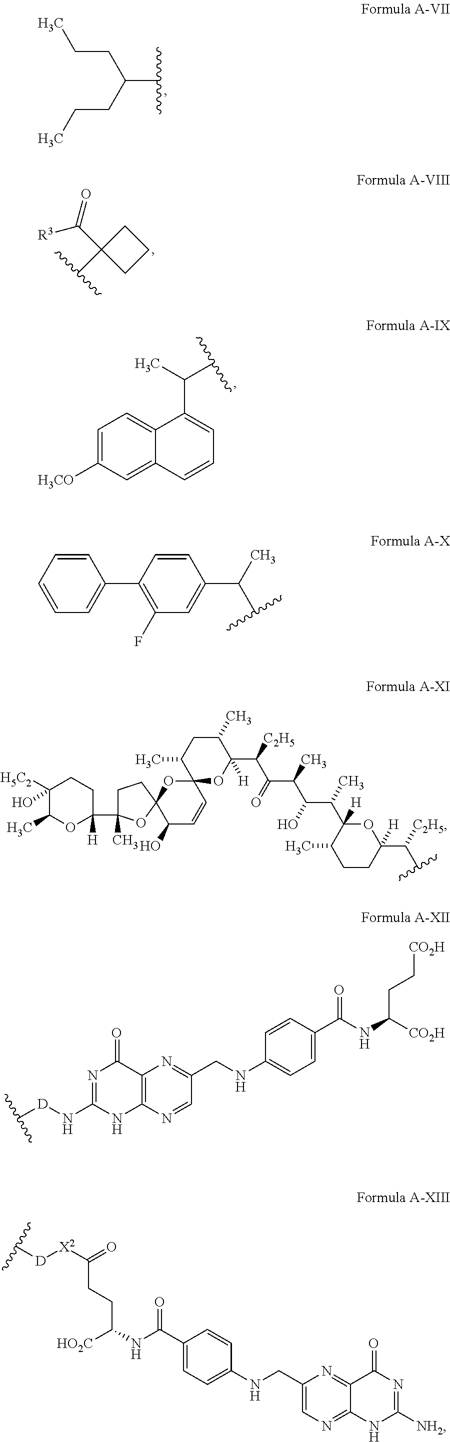 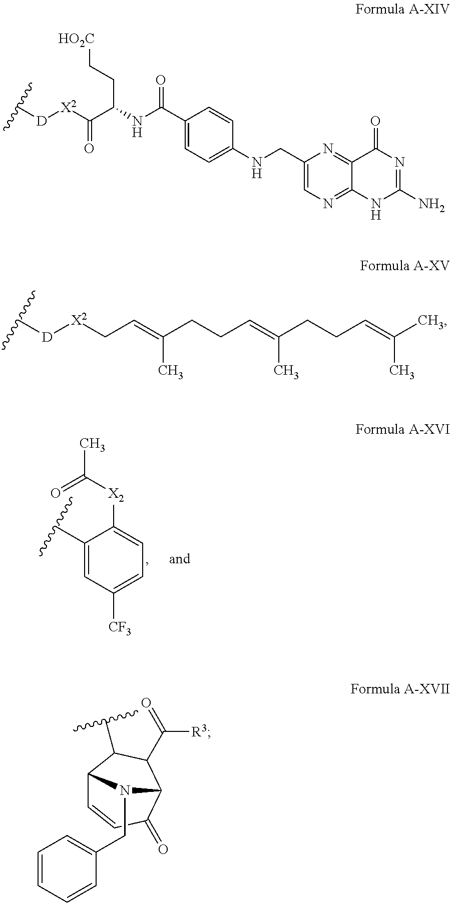
D is absent or 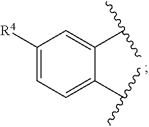
X¹ and X² are independently selected from —O—, —NR⁵—, and —S—;
R¹ and R⁴ are independently selected from hydrogen and trifluoromethyl;
R² is selected from —SCH₃, —S(O)CH₃, and —S(O)₂CH₃;
R³ is selected from hydroxyl, Z, and —X¹—B-Z;
R⁵ is selected from hydrogen and C₁₋₆ alkyl;
B is selected from: 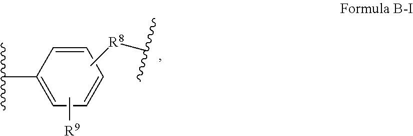
a single bond, and an aliphatic group with 1 to 22 carbon atoms;
R⁸ is a C₁₋₄ alkylene; and
R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;
or a pharmaceutically acceptable salt thereof.

5. The method of claim 4, wherein said compound is: 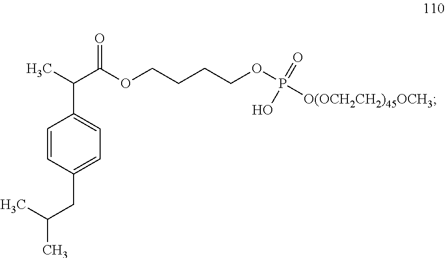
or a pharmaceutically acceptable salt thereof.

6. The method of claim 1, wherein said compound is a compound of Formula (III): 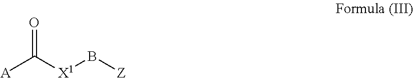
or a pharmaceutically acceptable salt thereof, wherein
A is selected from: 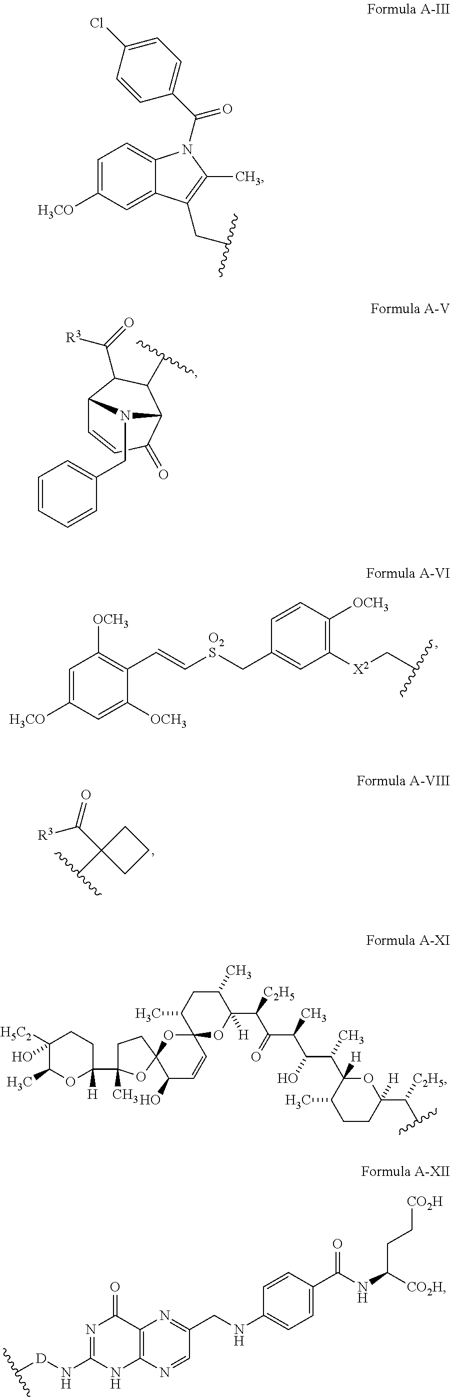 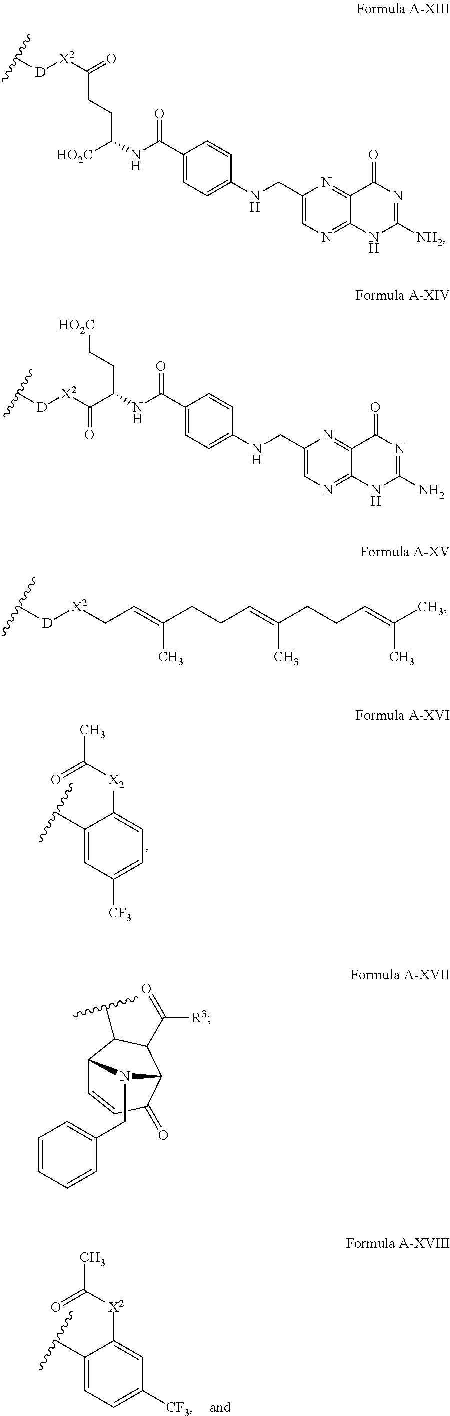 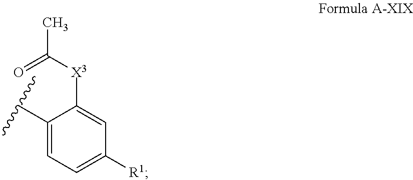
D is absent or 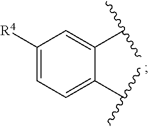
X¹ and X² are independently selected from —O—, —NR⁵—, and —S—;
R¹ and R⁴ are independently selected from hydrogen and trifluoromethyl;
X³ is selected from —S— and —NH—;
R³ is selected from hydroxyl, Z, and —X¹—B-Z;
R⁵ is selected from hydrogen and C₁₋₆ alkyl;
B is selected from: 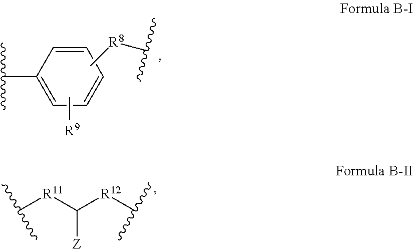
a single bond, and an aliphatic group with 1 to 22 carbon atoms;
R⁸, R¹¹, and R¹² are the same or different C₁₋₄ alkylene;
R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;
Z is selected from: 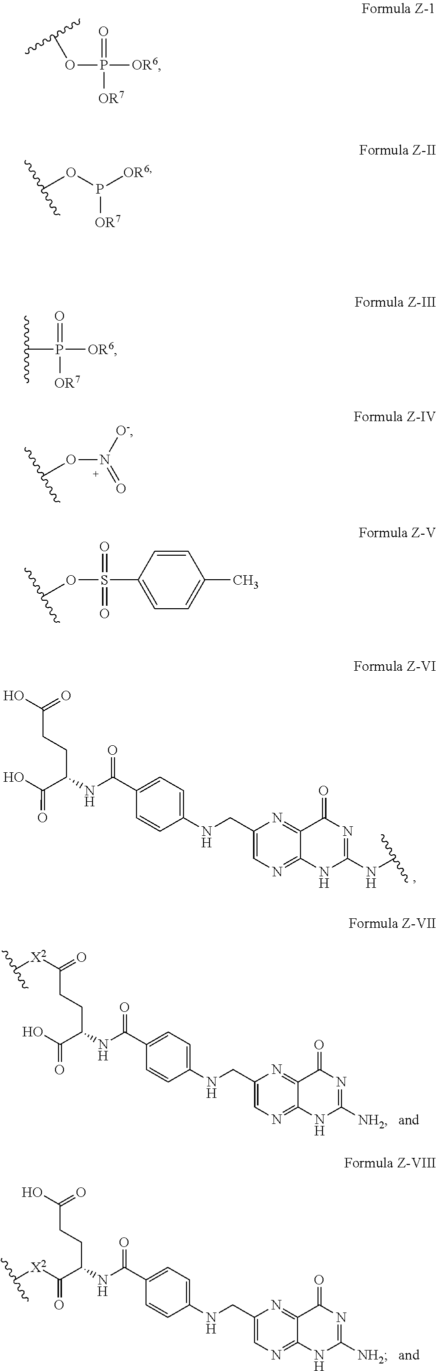
or B together with Z forms a structure: 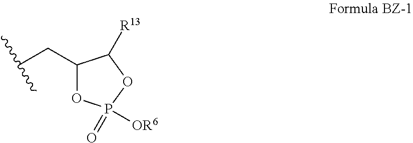
R⁶ and R⁷ are independently selected from hydrogen, C₁₋₆-alkyl, and polyethylene glycol residue; and
R¹³ is selected from hydrogen, an aliphatic group with 1 to 22 carbon atoms (e.g. C₁₋₆-alkyl), and polyethylene glycol residue;
or a pharmaceutically acceptable salt thereof.

7. The method of claim 6, wherein said compound is selected from compounds 22 to 92, 108, 111 to 116 or a pharmaceutically acceptable salt thereof.

8. The method of claim 1, wherein said compound is a compound of Formula (IV): 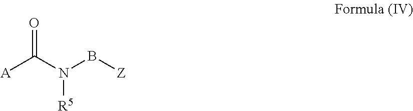
or a pharmaceutically acceptable salt thereof, wherein
A is an optionally substituted aliphatic, heteroaliphatic, aromatic, heteroaromatic substituent or alkylaryl substituent having 1 to 100 carbon atoms or selected from: 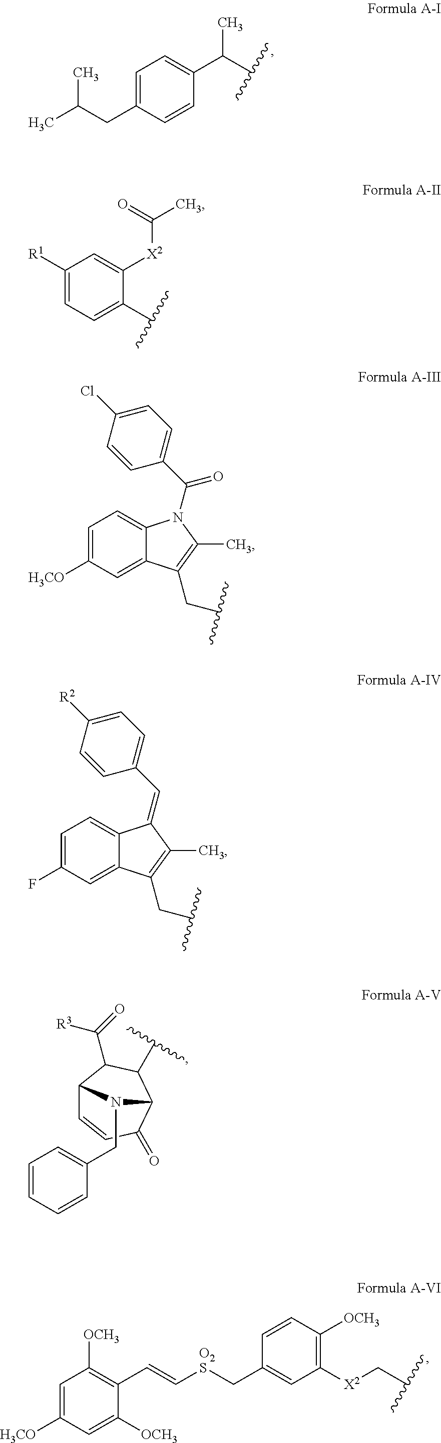 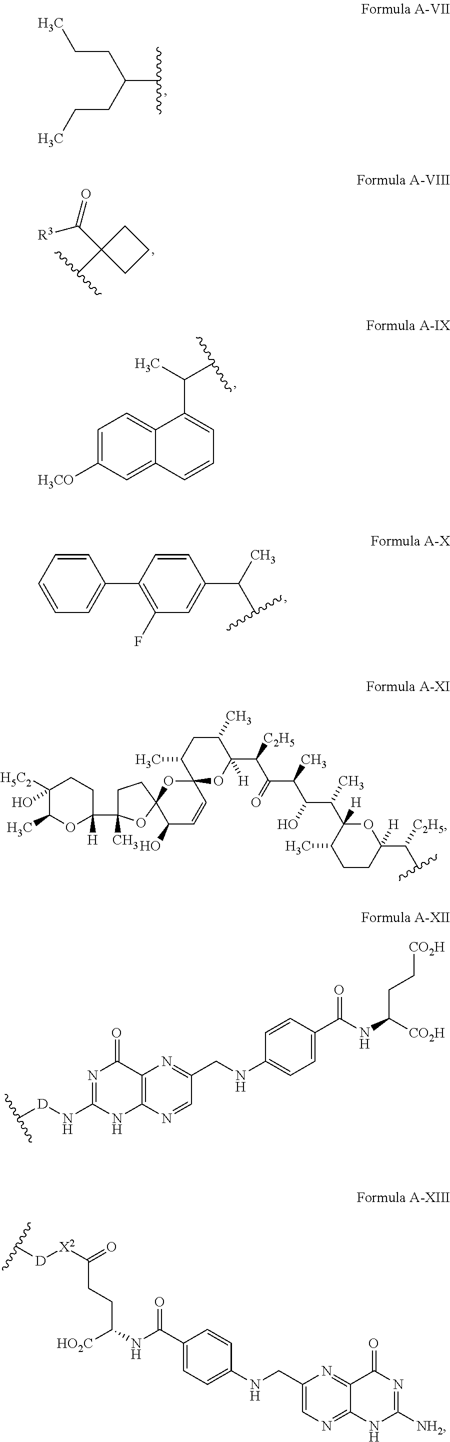 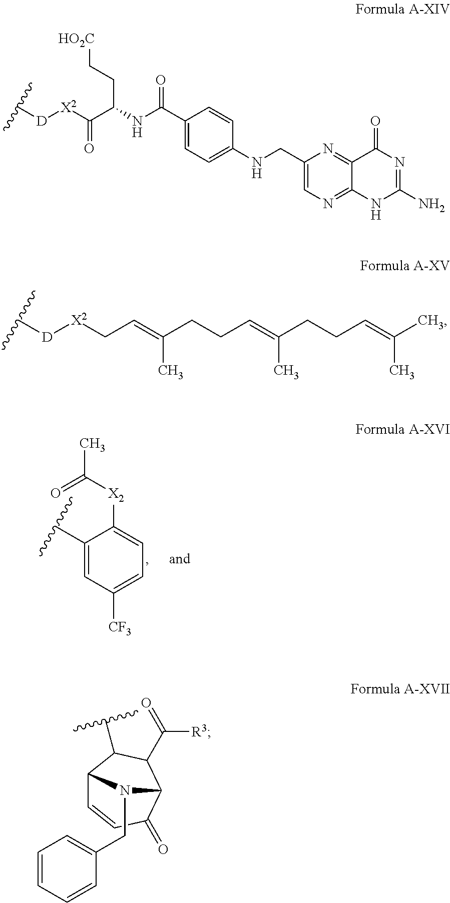
D is absent or 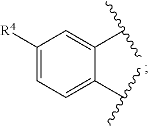
X² is selected from —O—, —NR⁵—, and —S—;
R¹ and R⁴ are independently selected from hydrogen and trifluoromethyl;
R² is selected from —SCH₃, —S(O)CH₃, and —S(O)₂CH₃;
R³ is selected from hydroxyl, Z, and —X¹—B-Z;
R⁵ is selected from methyl and ethyl;
B is selected from: 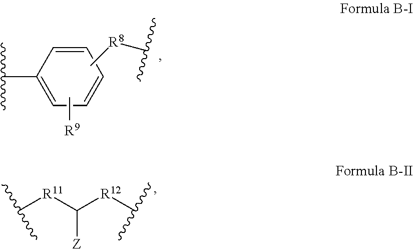
a single bond, and an aliphatic group with 1 to 22 carbon atoms;
R⁸, R¹¹, and R¹² are the same or different C₁₋₄ alkylene;
R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;
Z is selected from: 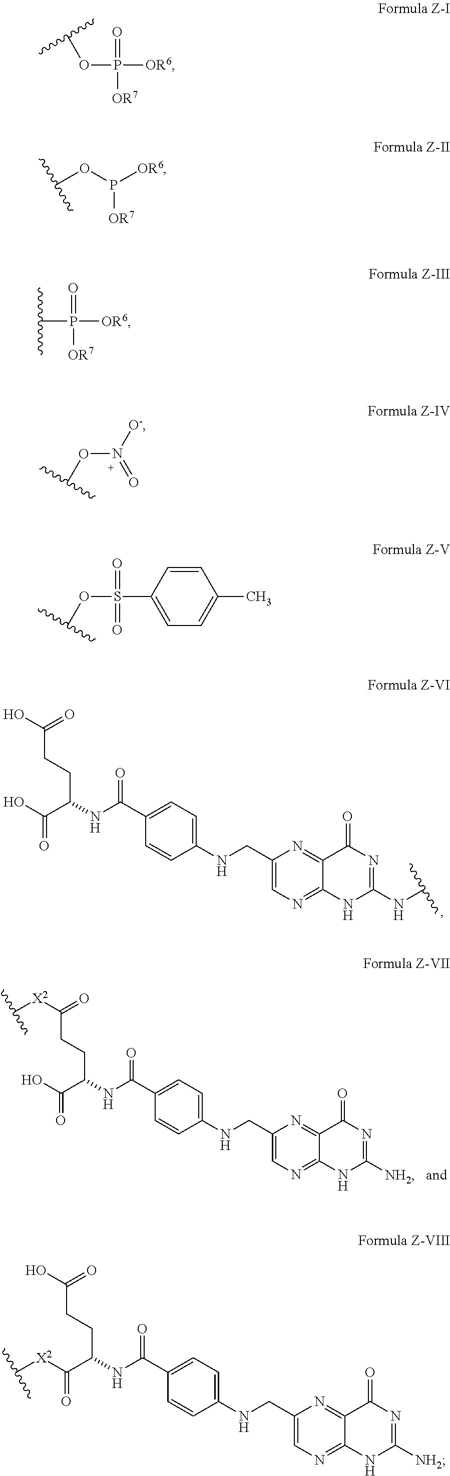
or B together with Z forms a structure: 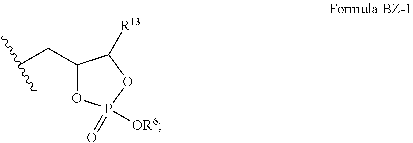
R⁶ and R⁷ are independently selected from hydrogen, C₁₋₆-alkyl, and polyethylene glycol residue; and
R¹³ is selected from hydrogen, an aliphatic group with 1 to 22 carbon atoms (e.g. C₁₋₆-alkyl) or polyethylene glycol residue.

9. The method of claim 1, wherein said compound is selected from compounds 93 to 107 and 117 to 134 or a pharmaceutically acceptable salt thereof.

10. The method of claim 1, further comprising administering difluoromethylornithine and/or cimetidine to the subject, where the compound and the difluoromethylornithine and/or cimetidine are administered within 24 hours of each other in amounts that together are effective to treat the subject.

11. The method of claim 10, wherein said compound and said difluoromethylornithine and/or cimetidine are formulated as a single pharmaceutical composition.

12. The method of claim 1, wherein the compound is administered to the subject in the form of a hydrogel or nanocarrier.

13. The method of claim 12, wherein the hydrogel comprises a poloaxamer and oleic acid.

14. The method of claim 1, wherein said non-cancerous condition of the skin and mucous membranes is selected from eczema, atopic dermatitis, dryness of the skin, recurring skin rash, contact dermatitis, dyshidrosis, xerotic eczema, seborrhoeic dermatitis, neurodermatitis, discoid eczema, venous eczema, actinic keratosis, cutaneous papilloma, anogenital papilloma, benign epithelial tumor, and hirsutism.
